(19) United States
(12) Patent Application Publication
(10) Pub. No.: US 2024/0156800 A1
(43) Pub. Date: May 16, 2024

(54) EP300 DEGRADER AND USES THEREOF IN NEUROBLASTOMA

(71) Applicant: DANA-FARBER CANCER INSTITUTE, INC., Boston, MA (US)

(72) Inventors: Jun Qi, Sharon, MA (US); Adam D. Durbin, Memphis, TN (US)

(73) Assignee: DANA-FARBER CANCER INSTITUTE, INC., Boston, MA (US)

(21) Appl. No.: 18/280,877

(22) PCT Filed: Mar. 8, 2022
(86) PCT No.: PCT/US2022/019309

§ 371 Date: Sep. 7, 2023

Related U.S. Application Data
(60) Provisional application No. 63/158,620, filed on Mar. 9, 2021.

(51) Int. Cl. A61K 31/454; A61K 45/06; A61P 35/00; C12Q 1/6886

(52) U.S. Cl. CPC A61K 31/454; A61K 45/06; A61P 35/00; C12Q 1/6886; C12Q 2600/106; C12Q 2600/158

(57) ABSTRACT

The present invention relates to methods for treating a disease or disorder associated with EP300 dependency and elevated CRBN expression levels (e.g., cancer (e.g., neuroblastoma)).

### RELATED APPLICATIONS

This application claims the benefit of priority under 35 U.S.C. § 119(e) to U.S. Provisional Application No. 63/158,620, filed Mar. 9, 2021, which is incorporated herein by reference in its entirety.

### SEQUENCE LISTING

The instant application contains a Sequence Listing which has been submitted electronically in ASCHII format and is hereby incorporated by reference in its entirety. Said ASCII copy, created on Mar. 8, 2022, is named 52095-7180001WO_ST25.txt and is 72 kilobytes in size.

### BACKGROUND OF THE INVENTION

Various studies have suggested that E1A-binding protein P300 (EP300, KAT3B) and cAMP responsive element binding protein (CREB)-binding protein (CBP, CREBBP, KAT3A) play overlapping but distinct roles in the regulation of cell survival. Germline loss of EP300 or CBP results in murine embryonic lethality with distinct phenotypes (Yao et al. Cell 93:361-72 (1998)). Furthermore, while CBP is required for self-renewal, EP300 is required for differentiation of hematopoietic stem cells (Rebel et al. Proc.Natl. Acad. Sci. U.S.A. 99:14789-94 (2002)). Somatic mutations of either EP300 or CBP are found in a variety of malignancies, including neuroblastoma, and the loss EP300 in CBP-mutated tumor cells is synthetically lethal (Barretina et al. Nature 483:603-7 (2012); Ogiwara et al. Cancer Discov. 6:430-45 (2016)).

Chromatin immunoprecipitation coupled to high-throughput sequencing (ChIP-Seq) studies have identified overlapping but distinct DNA binding sites for EP300 and CBP genome-wide, indicating that these two proteins may function differently by regulating the enhancers of distinct genes (Martire et al. BMC Mol. Cell Biol. 21:55 (2020); Ramos et al. Nucleic Acids Res. 38:5396-5408 (2010)). Many studies interrogating EP300 and CBP have relied on genetic disruption or mRNA depletion of each gene, which does not permit a time-associated analysis, or alternatively have relied on the use of inhibitors with non-selective activity against both enzymes (Dancy and Cole, Chem. Rev. 115:2419-52 (2015); Hammitzsch et al. Proc.Natl. Acad. Sci. U.S.A. 112:10768-173 (2015); Lasko et al. Nature 550:128-2 (2017); Yan et al. J. Invest. Dermatol. 133:2444-52 (2013); Zucconi et al. Biochemistry 55:3727-34 (2016)). The derivation of pharmacologic inhibitors targeting only one of these enzymes has thus been limited by the homology between these proteins (Dancy and Cole, Chem. Rev. 115:19-2452 (2015); Lasko et al. Nature 550:128-32 (2017)).

### SUMMARY OF THE INVENTION

The present invention is based upon the surprising discovery that EP300, but not its paralog CREB-binding protein (CBP), is required for regulation of key enhancers in high-risk neuroblastoma. EP300 is an enhancer-regulating dependency in neuroblastoma (NB), recruited to DNA through interactions with transcription factor activating protein 2B (TFAP2β), a member of the lineage-defining core-regulatory circuitry of high-risk neuroblastoma. Targeted pharmacologic degradation of EP300 by the proteolysis targeting chimera (PROTAC®) JQAD1 resulted in global loss of histone acetylation in neuroblastoma. Degradation of EP300 drives neuroblastoma apoptosis due in part to loss of MYCN chromatin localization and has limited toxicity to untransformed cells. Functional genomic and chemical analysis revealed widespread dependency on EP300 in many types of human cancers, for example, myeloma, lymphoma, leukemia, melanoma, rhabdomyosarcoma, colon cancer, rectum cancer, stomach cancer, breast cancer, brain cancer, and pancreatic cancer.

Methods of treating a subject, e.g., a human subject, with a disease or disorder associated with EP300 dependency and elevated cereblon (CRBN) expression levels are carried out by obtaining a test sample from a subject having or at risk of developing the disease; identifying increased expression level of CRBN in the test sample as compared to a reference sample; and administering to the subject a therapeutically effective amount of a selective degrader of EP300, thereby treating the disease or disorder.

In one aspect, the disease or disorder is a cancer. In certain embodiments, the cancer is solid tumor (i.e., a tumor lacking any liquid or cysts), for example, neuroblastoma, rhabdomyosarcoma, melanoma, colon cancer, rectum cancer, stomach cancer, breast cancer, brain cancer, and pancreatic cancer. In certain embodiments the cancer is a hematologic cancer (i.e., cancers affecting blood, bone marrow, and lymph nodes), for example, leukemia, myeloma, and lymphoma. In certain embodiments, the cancer is high-risk neuroblastoma.

For example, the test sample is obtained from a tumor tissue or a tumor microenvironment. Alternatively, the test sample is obtained from a bodily fluid, e.g., plasma, blood, urine, sputum, or cerebrospinal fluid (CSF). Other exemplary bodily fluids include serous fluids (e.g., pleural, peritoneal, and pericardial fluids), synovial fluid, and drainage and dialysis fluids.

In one aspect, the reference sample is obtained from healthy normal tissue or tumor tissue. For example, the reference sample is obtained from healthy normal tissue from the same individual as the test sample or one or more healthy normal tissues from different individuals.

In some cases, whether EP300 is required for tumor growth, i.e., whether the tumor is EP300 dependent, is identified by CRISPR-Cas9-mediated knockout of EP300 in the cells of a test sample.

In some cases, the expression level of CRBN is detected via an Affymetrix Gene Array hybridization, next generation sequencing, ribonucleic acid sequencing (RNA-seq), a real time reverse transcriptase polymerase chain reaction (real time RT-PCR) assay, immunohistochemistry (IHC), or immunofluorescence.

In one aspect, the selective degrader of EP300 is JQAD1 or a pharmaceutically acceptable salt thereof.

Preferably, tumor cell survival, tumor cell proliferation, or tumor metastasis is inhibited, e.g., by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

Optionally, tumor cell growth is reduced, e.g., by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. In another aspect, tumor cell apoptosis is induced.

In some cases, the methods further comprise administering to the subject a chemotherapeutic agent, radiation therapy, cryotherapy, hormone therapy, immunotherapy, or stem cell transplant. For example, the chemotherapeutic agent comprises cis-retinoic acid, cyclophosphamide (Cytoxan®, Neosar®, Endoxan®), cisplatin (Platinol®), carboplatin (Paraplatin®), vincristine (Oncovin®, Vincasar PFS®, VCR), doxorubicin (Adriamycin®, Rubex®), etoposide (Toposar®, VePesid®, Etopophos®, VP-16) , topotecan (Hycamtin®), busulfan (Myleran®, Busulfex®) and melphalan (Alkeran®, L-PAM, Evomela®), or thiotepa (Thioplex®, Tepadina®).

In one aspect, the chemotherapeutic agent is administered with a steroid. For example, the steroid is prednisone (Sterapred®, Prednisone Intensol) or dexamethasone (Decadron®).

In some cases, the methods further comprise administering to the subject a combination chemotherapy agent. For example, the combination chemotherapy agent includes carboplatin (Paraplatin®) or cisplatin (Platinol®), cyclophosphamide (Cytoxan®, Neosar®, Endoxan®), doxorubicin (Adriamycin®, Rubex®), and etoposide (Toposar®, VePesid®, Etopophos®, VP-16), or irinotecan (Onivyde®), temozolomide (Temodal®), or ifosfamide (Ifex®). In some cases, this treatment is followed by a stem cell transplant.

In some cases, the methods further comprise administering to the subject an immunosuppressant agent such dinutuximab (Unituxin®) with or without cis-retinoic acid.

Also provided are methods of determining whether degradation of EP300 in a subject with cancer will result in clinical benefit in the subject comprising: obtaining a test sample from a subject having or at risk of developing cancer; determining expression level of CRBN in the test sample; comparing the expression level of CRBN with the expression level of CRBN in a reference sample; and determining whether EP300 degradation will inhibit the cancer in the subject if the expression level of CRBN in the test sample differs from the expression level of the CRBN in the reference sample.

For example, the test sample is obtained from a tumor tissue or from a tumor microenvironment. Alternatively, the test sample is obtained from a bodily fluid, e.g., plasma, blood, urine, sputum, or CSF. Other exemplary bodily fluids include serous fluids (e.g., pleural, peritoneal, and pericardial fluids), synovial fluid, and drainage and dialysis fluids.

In one aspect, the reference sample is obtained from healthy normal tissue.

For example, clinical benefit in the subject comprises complete or partial response as defined by response evaluation criteria in solid tumors (RECIST), stable disease as defined by RECIST, or long-term survival in spite of disease progression or response as defined by irRC criteria.

In one case, the test sample is obtained from the cancer tissue, and the method further comprises determining that degradation of EP300 in a subject with cancer will result in clinical benefit in the subject if the expression level of CRBN in the test sample is equal to or higher than the level of CRBN in the reference sample.

In another case, the test sample is obtained from the cancer tissue, and the method further comprises determining that degradation of EP300 in a subject with cancer will not result in clinical benefit in the subject if the expression level of CRBN in the test sample is lower than the level of CRBN in the reference sample.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The phrase “aberrant expression” is used to refer to an expression level that deviates from (i.e., an increased or decreased expression level) the normal reference expression level of the gene.

By “agent” is meant any small compound, antibody, nucleic acid molecule, or polypeptide, or fragments thereof

By “alteration” is meant a change (increase or decrease) in the expression levels or activity of a gene or polypeptide as detected by standard art-known methods such as those described herein. As used herein, an alteration includes at least a 1% change in expression levels, e.g., at least a 2%, 3%, 4%, 5%, 6%, 7%, 8%, 9%, 10%, 20%, 30%, 40%, 50%, 60%, 70%, 80%, 90%, or 100% change in expression levels. For example, an alteration includes at least a 5%-10% change in expression levels, preferably a 25% change, more preferably a 40% change, and most preferably a 50% or greater change in expression levels.

The term “antibody” (Ab) as used herein includes monoclonal antibodies, polyclonal antibodies, multispecific antibodies (e.g., bispecific antibodies), and antibody fragments, as long as they exhibit the desired biological activity. The term “immunoglobulin” (Ig) is used interchangeably with “antibody” herein.

By “binding to” a molecule is meant having a physicochemical affinity for that molecule.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g., β-galactosidase or luciferase). Depending on the method used for detection, the amount and measurement of the change can vary. Determination of statistical significance is within the ability of those skilled in the art, e.g., the number of standard deviations from the mean that constitute a positive result.

As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease, e.g., NB, relative to an untreated patient. The term “therapeutically effective amount” includes the amount of the compound, alone or in a combination, which when administered, may induce a positive modification in the disease (e.g., NB) (e.g., to degrade EP300 in diseased cells), or is sufficient to prevent development or progression of the disease, or alleviate at least to some extent, one or more of the symptoms of the disease in a subject. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “expression profile” is used broadly to include a genomic expression profile. Profiles may be generated by any convenient means for determining a level of a nucleic acid sequence, e.g., quantitative hybridization of microRNA, labeled microRNA, amplified microRNA, complementary/synthetic DNA (cDNA), etc., quantitative polymerase chain reaction (PCR), and ELISA for quantitation, and allow the analysis of differential gene expression between two samples. A subject or patient tumor sample is assayed. Samples are collected by any convenient method, as known in the art. According to some embodiments, the term “expression profile” means measuring the relative abundance of the nucleic acid sequences in the measured samples.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity, e.g., at least 80%, at least 85%, at least 90%, at least 95%, or at least 99% identity. Polynucleotides having “substantial identity” to an endogenous sequence are typically capable of hybridizing with at least one strand of a double-stranded nucleic acid molecule.

As used herein, “obtaining” as in “obtaining an agent” includes synthesizing, purchasing, or otherwise acquiring the agent.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition, or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may further include one or more pharmaceutically acceptable excipients. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

By “protein” or “polypeptide” or “peptide” is meant any chain of more than two natural or unnatural amino acids, regardless of post-translational modification (e.g., glycosylation or phosphorylation), constituting all or part of a naturally occurring or non-naturally occurring polypeptide or peptide, as is described herein.

The terms “preventing” and “prevention” refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

The term “prognosis,” “staging,” and “determination of aggressiveness” are defined herein as the prediction of the degree of severity of the neoplasia, e.g., NB, and of its evolution as well as the prospect of recovery as anticipated from usual course of the disease. Once the aggressiveness has been determined, appropriate methods of treatments are chosen.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “selective degrader” is meant a bifunctional compound or PROTAC® (e.g., JQAD1) that preferentially binds and recruits a specific protein (e.g., EP330) for targeted proteasomal degradation.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with EP300 dependency and elevated CRBN expression levels (e.g., cancer (e.g., NB)) is within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

The terms “treating” and “treatment” as used herein refer to the administration of an agent or formulation to a clinically symptomatic individual afflicted with an adverse condition, disorder, or disease, so as to affect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition, or symptoms associated therewith be completely eliminated.

In some cases, a composition of the invention is administered orally or systemically. Other modes of administration include rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

In some embodiments, compositions of the invention may be administered orally to a subject in need thereof in the form of a capsule or tablet. In some embodiments, compositions of the invention may be administered parenterally to a subject in need thereof in the form of a liquid.

Pharmaceutical compositions may be assembled into kits or pharmaceutical systems for use in arresting cell cycle in rapidly dividing cells, e.g., cancer cells. Kits or pharmaceutical systems according to this aspect of the invention comprise a carrier means, such as a box, carton, or tube, having in close confinement therein one or more container means, such as vials, tubes, ampoules, bottles, syringes, or bags. The kits or pharmaceutical systems of the invention may also comprise associated instructions for using the kit.

Any compositions or methods provided herein can be combined with one or more of any of the other compositions and methods provided herein.

Where applicable or not specifically disclaimed, any one of the embodiments described herein are contemplated to be able to combine with any other one or more embodiments, even though the embodiments are described under different aspects of the invention.

These and other embodiments are disclosed and/or encompassed by the following Detailed Description.

### BRIEF DESCRIPTION OF THE DRAWINGS

FIG. 1A-FIG. 1D are a set of graphs, western blots, and a heatmap showing that E1A-binding protein (EP300), but not CREB-binding protein (CBP), is required for neuroblastoma cell growth. FIG. 1A is a heatmap of probability of dependency on neuroblastoma cell lines (n=19) in the DepMap 20Q2 data release. FIG. 1B is a set of western blots of Kelly cells stably expressing Cas9 that were infected with single guide RNAs (sgRNAs) targeting EP300 (EP300-1,2), CBP (CBP-1,2) or controls (ch2.2, LACZ). FIG. 1C is a graph of colony formation assays that were performed following sgRNA infection as in FIG. 1B in Kelly and BE2C cells. n=3 independent replicates per cell line, per treatment. *p<0.05. FIG. 1D is a graph of Kelly NB cells that were treated in colony formation assays with a range of concentrations of the EP300/CBP combined inhibitors C646, CBP30, and A485. n=3 independent replicates per cell line, per treatment. See also, FIG. 8A-FIG. 8N.

FIG. 2A-FIG. 2I are a set of heatmaps, chromatin immunoprecipitation sequencing (ChIP-seq) tracks, and western blots and graph showing that EP300 regulates the neuroblastoma core-regulatory circuitry directed by transcription factor activating protein 2B (TFAP2β). FIG. 2A is a STRING database interaction plot of nuclear dependency genes in neuroblastoma (NB) cells. Data is derived from Durbin et al. Nat Genet 50:1240-1246 (2018). Shown are core-regulatory circuitry members (blue), or proteins with enzymatic domains (red). FIG. 2B is a heatmap of ChIP-seq of EP300 and CBP in BE2C NB cells. Heatmap analysis was performed and ranked by the ratio of EP300:CBP binding. Data is representative of two cell lines, Kelly and BE2C. FIG. 2C is a genome-wide heatmap analysis of Assay for Transposase-Accessible Chromatin using sequencing (ATAC-seq) and ChIP-seq of EP300, H3K27ac and the core-regulatory circuitry factors at the union of core transcriptional regulatory circuitry (CRC) transcription factor binding sites in BE2C and NB cells, ranked by MYCN binding. Data is representative of two cell lines, Kelly and BE2C. FIG. 2D. is a set of representative ChIP-seq tracks demonstrating binding of CRC factors (blue), CBP (green), EP300 (red) at the HAND2 locus in Kelly NB cells. Also shown is the heart and neural crest derivatives expressed 2 (HAND2) super-enhancer (H3K27ac) and open chromatin (ATAC-seq) (black). Data is representative of both Kelly and BE2C cells. FIG. 2E is a graph of motif enrichment analysis of ChIP-seq of EP300 and CBP in Kelly NB cells. Data was restricted to the top 500 bound peaks by EP300 or CBP in Kelly NB cells. Colored dots indicate known enriched transcription factors. Arrow indicates enriched motif, corresponding to TFAP2β. FIG. 2F is position-weight matrix from analysis in FIG. 2D demonstrating the top enriched sequence under EP300 peaks, compared with CBP peaks, which corresponds to the consensus binding sequence for TFAP2β. FIG. 2G is a western blot of co-immunoprecipitation of EP300 and CBP in Kelly NB cells. WCL=whole cell lysate. IgG=isotype-matched rabbit IgG antibody. Data is representative of three independent western blots. FIG. 2H is a western blot of Kelly NB cells expressing Cas9 that were infected with sgRNAs targeting TFAP2β (TFAP2β-1,2) or control loci (ch2.2, LACZ). Data is representative of three independent lysates and blots. FIG. 2I is graph of Propidium-iodide flow cytometry of Kelly NB cells expressing Cas9 and infected with sgRNAs targeting TFAP2β (TFAP2β-1,2) or control loci (ch2.2, LACZ). n=3 independent infections and flow analyses. *p<0.05. See also FIG. 9A-FIG. 9K.

FIG. 3A-FIG. 3I are a set of chemical structures, graphs, and western blots showing that JQAD1 is a selective EP300 degrader. FIG. 3A is an image of the chemical structures of (R,S)-A485 and (R,S)-JQAD1 highlighting the structural components of JQAD1. FIG. 3B is graph of a CellTiter-Glo® assay of Kelly cells that were treated with 1 μM (R,S)-A485, (R,S)-JQAD1, (S,S)-JQAD1 or dimethyl sulfoxide (DMSO) for 6 days. n=3 independent experiments and measurements at each time point. FIG. 3C is a graph of an AlphaLISA® assay of multiple immunomodulatory imide drug (iMiD) containing molecules (pomalidomide, thalidomide, lenalidomide, and JQAD1) and A485. Data is representative of three independent assays. FIG. 3D is a western blot of Kelly cell lysates that were treated with combinations of Biotin-JQAD1 or pomalidomide, prior to streptavidin-bead purification. WCL=whole cell lysate. Data is representative of three independent experiments and blots. FIG. 3E is a set of western blots of Kelly NB cells that were treated with DMSO, A485 or JQAD1 at the noted concentrations (in μM) for 24 hours (h) prior to lysis. Data is representative of three independent experiments and blots. FIG. 3F is a graph of stable isotope labeling using amino acids in cell culture (SILAC) labelled-Kelly NB cells that were treated with 500 nM JQAD1 or DMSO vehicle for 24 h prior to nuclear extraction and analysis by mass spectrometry. Ratio of detected peptides at 0 h vs. 24 h is demonstrated. Data represents the sum ratio of heavy:light labelled protein detected in triplicate at 24 h compared to 0 h. Dotted line indicates a p-value of 0.01. Red labelled points indicate EP300 and CBP. n=3 independent treatments, lysates, and mass spectrometry reactions. FIG. 3G is a western blot of Kelly NB cells that were treated with 500 nM JQAD1 for the noted time points prior to lysis. Data is representative of three independent experiments and blots. FIG. 3H is a graph of propidium iodide (PI)-flow cytometry of sub-G1 events in Kelly (H) cells treated with JQAD1 or A485 for the noted concentrations and time points. Data is a summary of n>3 independent flow experiments. Similar results were obtained in SIMA cells treated with compounds at 1 μM. FIG. 31 is a graph of PI-flow cytometry of sub-G1 events in Kelly (H) and NGP (I) cells treated with JQAD1 or A485 for the noted concentrations and time points. Data is a summary of n>3 independent flow experiments. See also FIG. 10A-FIG. 10N.

FIG. 4A-FIG. 4E are a set of western blots and plots showing that JQAD1, but not A485, disrupts MYCN chromatin localization resulting in apoptosis. FIG. 4A is a western blot of markers of apoptosis cleaved caspase-3 and cleaved PARP1 in Kelly NB cells that were treated with 1 μM JQAD1, A485 or DMSO control for 12, 24, and 36 h prior to lysis. Actin is demonstrated as a loading control. Data is representative of three independent treatment and analyses in Kelly and NGP cells. FIG. 4B is a plot of gene set enrichment analysis of RNAseq Kelly cells were treated with 500 nM JQAD1, A485 or DMSO control for 24 h prior to External RNA Controls Consortium (ERCC)-controlled spike in RNAseq. Gene set enrichment analysis of RNAseq results was performed, with the MSigDB Hallmarks dataset. n=3 biological replicates and independent RNA extractions per treatment. FIG.4C is a plot of normalized RNAseq gene expression of pro-and anti-apoptotic mRNA transcripts in Kelly cells that were treated with 500 nM JQAD1, A485 or DMSO control for 24 h. Log10 transcript abundance is shown, normalized against DMSO and ERCC controls. n=3 biological replicates and independent RNA extractions per treatment. FIG. 4D is a western blot of nuclear lysates of Kelly cell that were prepared and immunoprecipitated with anti-EP300, anti-CBP or IgG control antibodies. WCL=whole cell lysate. Data is representative of >3 independent co-immunoprecipitation and western blots. FIG. 4E is a western blot of Kelly cells that were treated with DMSO control, A485 (0.5, 1 μM) or JQAD1 (0.5, 1 μM) followed by extraction of chromatin. Total H3 is shown as a loading control. Data is representative of 3 independent treatments, lysates, and blots.

FIG. 5A-FIG. 5D are a set of plots and ChIP-seq tracks showing that JQAD1 caused loss of histone H3K27-acetylation, predominately at super-enhancers. FIG. 5A is a set of plots of enhancers ranked by H3K27ac signal at 0 h (left) and 24 h (right) after treatment of Kelly cells with 500 nM JQAD1. Data is representative of two independent treatments and ChIP-seq experiments. FIG. 5B is a set of plots of Log2 fold change in enhancer H3K27ac signal resolved by H3K27ac ChIP-seq in Kelly NB cells at 0 vs. 6 h (left) and 0 vs. 24 h (right). Data is normalized against external Drosophila melanogaster chromatin. FIG. 5C is a plot of Log2 fold change in enhancer H3K27ac signal stratified by super-enhancers and typical enhancers at 6 h and 24 h after treatment of Kelly cells with 500 nM JQAD1. *** indicates p<0.0001 by students t-test, comparing super-enhancer and typical enhancer-regulated genes at 24 h. FIG. 5D is set of representative gene tracks of Kelly cells treated with JQAD1 at 500 nM for 0 and 24 h at the HAND2 core-regulatory circuitry factor locus. Data is representative of the adrenergic CRC factor loci (HAND2, ISL1, PHOX2B, GATA3, TBX2, ASCLI and TFAP2E) and two independent treatments and ChIP-seq experiments. See also FIG. 11A-FIG. 11B.

FIG. 6A-FIG. 6E are a set of plots and immunohistochemistry images showing that JQAD1 caused tumor growth suppression and loss of EP300 in vivo. FIG. 6A is a plot of Kelly NB cell xenografts that were established in NOD scid gamma (NSG™) mice and mice treated with vehicle control (n=11), or JQAD1 at 40 mg/Kg intraperitoneally (IP) daily (n=12) or twice daily (n=12). **p<0.01, ***p<0.001 by two-way analysis of variance (ANOVA) with post-hoc Tukey test. Tumor growth curve kinetics were also analyzed by two-way ANOVA with mixed-effects analysis. FIG. 6B is plot of Kaplan-Meier survival analysis of mice described in FIG. 6B is a plot of JQAD1 at 40 mg/Kg IP twice daily prolongs survival, log-rank test p<0.0001 for both JQAD1 treated groups compared with vehicle. FIG. 6C is a plot of body weights of mice described in FIG. 6B-FIG. 6D. FIG. 6D is a set of immunohistochemistry images of EP300 and CBP in Kelly cell xenografts treated with either vehicle control or JQAD1 (40 mg/Kg IP daily) for 14 days. Data is representative of 3 independent animals per treatment. Bar=50 μm. FIG. 6E is a plot of ERCC-spike in RNA-seq, performed on tumor cells recovered from animals treated as-described in FIG. 6D, showing fold change in expression of animals treated with 40 mg/Kg JQAD1 daily (n=3) compared with vehicle control (n=4) at day 14. RNA-seq groups of genes are stratified by regulation by typical or super-enhancers, and gene identity of transcription factor or CRC gene. ***p<0.0001, *p=0.0223, ***p=0.0013, compared to CRC gene expression. See also FIG. 12A-FIG. 12D.

FIG. 7A-FIG. 7F are a set of plots and a western blot showing that cancer cells displayed biased dependency on EP300, compared to CBP. FIG. 7A is a plot comparing EP300 and CBP probability of dependency of all cell lines in DepMap (n=757, 20Q2 release). ***p<0.0001 by two-tailed Student's t-test. FIG. 7B is a set of plots of average EP300 (black) and CBP (red) probability of dependency of indicated cell lines. FIG. 7C is plot of area under the curve (AUC) of the dose-response relationship of barcoded cancer cell lines (n=557) that were treated with a concentration range of (R,S)-JQAD1 for 5 days prior to DNA extraction and resolution of survival. Cell lines were individually classified into lineages. Red bars=median, individual black dots=individual cell lines, red dots=neuroblastoma cell lines. AUC was calculated from triplicate measurements at each dose at time=120 h. FIG. 7D is a plot of JQAD1 AUC values from FIG. 7C against CRBN expression from the Cancer Cell Line Encyclopedia (CCLE). ***p<0.001 by ANOVA for >5 TPM compared against <4 and 4-5 TPM with post-hoc Bonferroni correction. FIG. 7E is a western blot of BE2C cells stably expressing control (zsGreen) or CRBN (CRBN) that were treated with DMSO or 10 μM JQAD1 for 2h. Cell lysates were subjected to western blotting for EP300, CBP and CRBN. Actin is shown as a loading control. Data is representative of three independent treatments and analyses. FIG. 7F is a plot of BE2C cells stably expressing control (zsGreen) or CRBN (CRBN) that were treated with DMSO or 10 μM JQAD1 for 6 days prior to by Cell-Titer Glo® analysis for cell growth. Data was normalized against BE2C-zsGreen, DMSO treated cells. ***p=0.008 by student's T-test comparing BE2C-CRBN DMSO and JQAD1 treated cells. n=3 biological replicates.

FIG. 8A-FIG. 8N is a set of western blots and plots showing EP300 and CBP expression are highly correlated in neuroblastoma, and EP300 is required for neuroblastoma cell survival. FIG. 8A is a western blot of BE2C cells expressing Cas9 that were infected with sgRNAs targeting EP300 (EP300-1,2), CBP (CBP-1,2) or controls (ch2.2, LACZ). Data is representative of three independent sgRNA infections and lysates. FIG. 8B is a plot of EP300 and CBP mRNA expression that were identified in primary neuroblastoma tumor samples. n=649, data retrieved from the R2 genomics browser, Kocak neuroblastoma dataset (Kocak et al. Cell Death Dis 4:e586 (2013)). FIG. 8C is a western blot of expression of EP300, CBP and CRBN in neuroblastoma cell lines. Total H3 is shown as a loading control. Data is representative of three independent lysates. FIG. 8D is a plot of mass-spectrometry-derived protein expression of EP300 and CBP retrieved from the DepMap portal, as described in Nusinow et al. Cell 180:387-402 (2020). n=375 cancer cell lines. FIG. 8E is a plot of expression of CBP and EP300 (TPM) across the cancer cell line encyclopedia (n=1371), as described in Ghandi et al. Nature 569:503-8 (2019). FIG. 8F-FIG. 8H are a set of plots of colony formation assays of BE2C (FIG. 8F), NB69 (FIG. 8G) and NGP (FIG. 8H) cells that were treated with range concentrations of combined EP300/CBP inhibitors C646, CBP30 and A485. n=3 independent treatments per data point. FIG. 8I-FIG. 8K are a set of plots of PI-flow cytometry analysis of BE2C (FIG. 81), Kelly (FIG. 8J), NGP (FIG. 8K) cells were treated with DMSO, A485, C646 or CBP30 for 24h. n=3 independent treatments per cell lines. *p<0.05. A485 was used at 500 nM (Kelly), 1 μM (BE2C, NGP). CBP30 was used at 1 μM (Kelly, BE2C), 2.5 μM (NGP). C646 was used at 2.5 μM (Kelly), 5 μM (BE2C, NGP). FIG. 8L-FIG. 8M are a set of plots of PI-flow cytometry analysis of Kelly (FIG. 8L) and BE2C (FIG. 8M) cells expressing Cas9 were infected with lentiviruses expressing sgRNAs targeting EP300 (EP300-1,2), CBP (CBP-1,2) or controls (ch2.2, LACZ) for 5 days. n=3 independent infections and flow analyses. *p<0.05. FIG. 8N is a western blot of BE2C and Kelly NB cells that were treated with DMSO or A485 (BE2C 1 μM, Kelly 500 nM) for 7days prior to lysis. Data is representative of three independent lysates and blots.

FIG. 9A-FIG. 9K is set of heatmaps, plots, ChIP-seq tracks, and western blots showing that EP300 is recruited to CRC loci through physical interactions with the CRC member TFAP2β. FIG. A is a heatmap of ChIP-seq of EP300 and CBP in Kelly NB cells (ranked by the ratio of EP300:CBP binding). Data is representative of two cell lines, Kelly and BE2C. FIG. 9B is a genome-wide heatmap of ATAC-seq and ChIP-seq of EP300, H3K27ac and the core-regulatory circuitry factors at the union of CRC transcription factor binding sites in Kelly NB cells, ranked by MYCN binding. Data is representative of two cell lines, Kelly and BE2C. FIG. 9C is a set of ChIP-seq tracks of Kelly cells demonstrating binding of EP300 and CBP at core-regulatory circuitry loci and other neuroblastoma-relevant loci marked by typical enhancers (top panel) and super-enhancers (bottom panel). Data is representative of Kelly and BE2C cells. FIG. 9D is a plot of area under the curve analysis of motifs enriched under top 500 unique EP300 and CBP peaks in BE2C cells. Arrowhead indicates motif corresponding to TFAP213 that is enriched under EP300 peaks in both Kelly and BE2C cell lines. FIG. 9E is a set of position weight matrices of enriched sequences corresponding to known transcription factors in area under the ROC curve (AUROC) analysis. FIG. 9F is a plot of distribution of 35 proteins identified in both Kelly and BE2C cells after co-immunoprecipitation of H3K27ac from nuclear lysates and mass spectrometry analysis. Normal rabbit IgG was immunoprecipitated as a negative control. Data represents proteins identified in both Kelly and BE2C cells, with two independent co-IP-mass spectrometry reactions each. FIG. 9G is an immunoprecipitation-western blot data of EP300 and CBP in Kelly NB cells. WCL=whole cell lysate. IgG=isotype matched rabbit IgG control. Data is representative of three independent blots. FIG. 9H is a western blot of Kelly NB cells expressing Cas9 that were infected with lentiviruses expressing sgRNAs against TFAP213 (TFAP213-1-4), or control loci (ch2.2, LACZ). Data is representative of three independent blots. FIG. 9I-FIG. 9J are a set of western blots of Kelly NB cells expressing Cas9 that were infected with lentiviruses expressing sgRNAs against GATA3 (GATA3-1,2) (FIG. 91), HAND2 (HAND2-1,2) (FIG. 9J), or control loci (ch2.2, LACZ). Data is representative of three independent blots. FIG. 9K is plot of PI-flow cytometry of NGP cells expressing Cas9 that were infected with lentiviruses expressing sgRNAs against TFAP 213 (TFAP213-1,2), or control loci (ch2.2, LACZ). n=3 independent infections and PI-flow analyses. *p<0.0.5 compared to control.

FIG. 10A-FIG. 10N are a set of plots and western blots showing that (R,S)-JQAD1 is a selective CRBN-dependent EP300 degrader that inhibits NB cell growth. FIG. 10A-FIG. 10C are a set of plots of dose-viability measurements of Kelly (FIG. 10A), NGP (FIG. 10B) and SIMA (FIG. 10C) NB cells in response to (S,S)-JQAD1, (R,S)-JQAD1 and (R,S)-A485 after 6 days. Data was resolved by Cell-Titer Glo0 assay, and n=3 independent measurements per time point and dose. FIG. D-FIG. 10F are a set of plots of growth curves of Kelly (FIG. 10D), NGP (FIG. 10E) and SIMA (FIG. 10F) NB cells in response to DMSO, (S,S)-JQAD1, (R,S)-JQAD1 and (R,S)-A485 over 6 days of treatment. Compound doses used were 1 μM (Kelly), 2.5 μM (NGP) and 1 μM (SIMA). Data was resolved by Cell-Titer Glo® assay, and n=3 independent measurements per time point and dose. FIG. 10G is a western blot of Kelly NB cells that were treated with (R,S)-JQAD1 and (S,S)-JQAD1 for 24 h prior to lysis. Data is representative of three independent western blots. FIG. 10H is a western blot of a timecourse of NGP and SIMA cells treated with JQAD1 for 0 to 48 h prior to lysis. (R,S)-JQAD1 was used at 2.5 μM (NGP) and 1 μM (SIMA). Data is representative of three independent western blots. *denotes cleaved poly [ADP-ribose] polymerase 1 (PARP1). FIG. 10I is a western bot of Kelly cells stably expressing Cas9 that were infected with sgRNAs targeting CRBN (CRBN-1,3) or control loci (ch2.2, LACZ) and pools of knockout cells established. Western blotting was performed with antibodies against CRBN. Data is representative of three independent western blots. FIG. 10J-FIG. 10K is a set of plots of CellTiter-Glo® assays of Kelly-Cas9 control or CRBN knockout cells that were treated with a range of doses of JQAD1 (FIG. 10J) or A485 (FIG. 10K) for seven days. n=3 independent replicates per dose and time point. FIG. 10L is a western blot of Kelly-Cas9 control (ch2.2, LACZ) and CRBN knockout cells that were treated with DMSO or (R,S)-JQAD1 for 24 h at 500 nM. Data is representative of three independent treatments and lysates. *denotes cleaved PARP1. FIG. 10M is a western blot of Kelly cells that were pre-treated with DMSO, A485 (10 μM), Pomalidomide (20 μM), Bortezomib (2.5 nM), or MLN4924 (1 μM) for 4 h, followed by treatment with DMSO or JQAD1 for 48 h (500 nM). Data is representative of three independent blots. FIG. 10N is set of plots of PI-flow cytometry of G1, S and G2/M cell cycle phases in Kelly (top), NGP (middle) and SIMA (bottom) cells, treated with DMSO (white), (R,S)-A485 (blue) or (R,S)-JQAD1 (red) for 0, 24 and 48 h. Treatments were 500 nM (Kelly), 1 μM (NGP) and 1 μM (SIMA). Data is a summary of n>3 independent flow experiments. ***q<0.001, **q<0.01, *q<0.05 compared to 0 h controls by ANOVA with post-hoc multiple comparison correction.

FIG. 11A-FIG. 11B are a set of ChIP-seq tracks and a western blot showing that JQAD1 caused apoptosis and loss of core-regulatory circuitry gene locus enhancer acetylation. FIG. 11A is a western blot of NGP NB cells that were treated with 2.5 μM JQAD1, A485 or DMSO control for 12, 24, and 36 h. Data is representative of three independent treatments and analyses. FIG. 11B is a set of representative gene tracks of Kelly cells treated with (R,S)-JQAD1 at 500 nM for 0 and 24 h at core-regulatory circuitry factor loci. Data is representative of two independent treatments and ChIP-seq experiments.

FIG. 12A-FIG. 12D are a set of plots and immunohistochemistry images showing that JQAD1 is effective in vivo with limited toxicity. FIG. 12A is a plot of pharmacokinetic analysis of JQAD1 after a single intraperitoneal dose at 10 mg/Kg in CD1 mice. Half-life=13.3 (+/−3.37), Cₘₐₓ=7 μM. n=3 mice. FIG. 12B is a plot of daily dosing of JQAD1 at increasing doses in CD1 mice displaying no effect on animal weight. n=3 animals per group, with serial weight measurements. FIG. 12C is a plot of Balb/c CRBN^{ILE391VAL}-humanized knockin mice that were treated with vehicle or JQAD1 for 21 days at 40 mg/Kg IP daily, and their weight was measured daily (n=3 mice per treatment group). FIG. 12 D. is a set of immunohistochemistry images of formalin fixed mouse liver tissue sections that were stained with hematoxylin and eosin to assess possible toxicity and the effects of JQAD1 on the expression levels of EP300 and CBP. Three mice per treatment group were sacrificed after 14 days of treatment. Data is representative of three independent animals per treatment (vehicle, JQAD1). Bar=50 μm.

### DETAILED DESCRIPTION OF THE INVENTION

The present invention is based upon the surprising discovery that E1A-binding protein (EP300), but not its paralog CREB-binding protein (CBP), is required for regulation of key enhancers in high-risk neuroblastoma (NB). EP300 is an enhancer-regulating dependency in NB, recruited to DNA through interactions with transcription factor activating protein 2B (TFAP2β), a member of the lineage-defining core-regulatory circuitry of high-risk NB. Targeted pharmacologic degradation of EP300 by proteolysis targeting chimera (PROTAC®) JQAD1 resulted in global loss of histone acetylation in high-risk NB. Degradation of EP300 drives apoptosis due in part to loss of MYCN chromatin localization and has limited toxicity to untransformed cells. Functional genomic and chemical analysis revealed widespread dependency on EP300 in many types of human cancers, for example, myeloma, lymphoma, melanoma, rhabdomyosarcoma, colon cancer, rectum cancer, stomach cancer, breast cancer, brain cancer, and pancreatic cancer.

### High-Risk Neuroblastoma

High-risk NB is a pediatric tumor of the peripheral sympathetic nervous system derived from primitive neural crest cells, and which has a poor survival rate. These neuroendocrine tumors are characterized by high expression of oncogenic MYC family members. (Matthay et al. Nat. Rev. Dis. Primers 2:16078 (2016); Zimmerman et al. Cancer Discov. 8:320-35 (2018)). MYCN is an integral member of a positive feed-forward autoregulatory loop of transcription factors (TFs) that establish cell fate in MYCN-amplified NB. This group of TFs is termed the core-regulatory circuitry (CRC), and each member is regulated by a super-enhancer (SE) gene which is critically required for NB viability. One mechanism by which the MYC family oncogenes drive tumor growth is by invading gene enhancers and recruiting transcriptional and epigenetic machinery (Zeid et al. Nat. Genet. 50:515-23 (2018)). Combination pharmacologic inhibition of SE-mediated transcriptional initiation and elongation have been shown to rapidly disrupt the NB CRC in vitro and in vivo, resulting in transcriptional collapse and apoptosis (Durbin et al. Nat. Genet. 50:1240-6 (2018)).

Despite the fact that mass screening of NB does not significantly improve outcome for patients, some success in NB therapy has been achieved in recent years (Arakwa et al. J. Pediatr. 165:855-7 (2014)). NB grows and reacts differently to treatment in different subjects. NB is classified into 1 of 4 categories: very low-risk, low-risk, intermediate-risk, or high-risk by the International Neuroblastoma Risk Group (INRG) classification system. While patients with low- and intermediate-risk neuroblastoma have favorable prognosis and an excellent five-year survival rate of more than 90%, the prognosis of high-risk neuroblastoma (HR-NB), which is detected in approximately 60% of cases, remains unfavorable (Kholodenko et al. J. Immunol. Res. 2018:7394268 (2018)). The five-year survival rate remains under 50% despite aggressive multimodal therapy (Whittle et al. Expert Rev. Anticancer. Ther. 17:369-86 (2017)). The standard methods of neuroblastoma therapy have strong side effects, including serious damage to internal organs, anemia, effects on fertility, and hair loss. Chemotherapy, radiotherapy, and surgical methods demonstrate particularly low efficacy on the late stages of treatment of the disease, and they do not solve the problem of minimal residual disease, which is the cause of subsequent relapse (Kholodenko et al. J. Immunol. Res. 2018:7394268 (2018)).

### EP300 and CBP

EP300 and CBP are paralogous, multi-domain protein acetyltransferases with broad cellular functions mediated by protein-protein interactions and catalytic acetyltransferase activities (Dancy and Cole, Chem. Rev. 115:2419-52 (2015)). These proteins are independently mutated or translocated in a variety of human cancers, and numerous studies have identified distinct but overlapping activities of these proteins in untransformed cell types, including embryonic and hematopoietic stem cells and more differentiated fibroblasts and T-cells (Kasper et al. Mol. Cell. Biol. 26:789-809 (2006); Liu et al. Nat. Med. 19:1173-7 (2013); Rebel et al. Proc.Natl. Acad. Sci. USA 99:14789-94 (2002); Sen et al. Mol. Cell. 73:684-98 (2019); Yao et al. Cell 93:361-72 (1998)). EP300 and CBP display overlapping, but distinct binding patterns across the genome, indicating that these proteins exhibit only partial functional redundancy in transcriptional regulation (Martire et al. BMC Mol. Cell. Biol. 21:55 (2020); Ramos et al. Nucleic Acids Res. 38:5396-5408 (2010)). Due to the high degree of homology between these proteins, especially in the HAT and bromodomains, it has been difficult to design small molecule inhibitors that are selective for either one of these proteins. To this end, studies have demonstrated that EP300 exhibits synthetic lethality in cell lines in which CBP is mutationally inactivated (Ogiwara et al. Cancer Discov. 6:430-45 (2016)). However, both enzymes are expressed in most cell lines and primary tissues, making it difficult to distinguish between the functions of these two proteins.

| An exemplary EP300 amino acid sequence is provided at NCBI Accession No. | |
|---|---|
| NP_001349772, version NP_001349772.1, as set forth below (SEQ ID NO: 1): | |
| 1 | maenvvepgp psakrpklss palsasasdg tdfgslfdle hdlpdelins telgltnggd |
| 61 | inqlqtslgm vqdaaskhkq lsellrsgss pnlnmgvggp gqvmasqaqq sspglglins |
| 121 | mvkspmtqag ltspnmgmgt sgpnqgptqs tgmmnspvnq pamgmntgmn agmnpgmlaa |
| 181 | gngqgimpnq vmngsigagr grqnmqypnp gmgsagnllt eplqqgspqm ggqtglrgpq |
| 241 | plkmgmmnnp npygspytqn pgqqigasgl glqiqtktvl snnlspfamd kkavpgggmp |
| 301 | nmgqqpapqv qqpglvtpva qgmgsgahta dpekrkliqq qlvlllhahk cqrregange |
| 361 | vrqcnlphcr tmknvlnhmt hcqsgkscqv ahcassrqii shwknctrhd cpvclplkna |
| 421 | gdkrnqqpil tgapvglgnp sslgvgqqsa pnlstvsqid pssierayaa lglpyqvnqm |
| 481 | ptqpqvgakn qqnqqpgqsp qgmrpmsnms aspmgvnggv gvqtpsllsd smlhsainsq |
| 541 | npmmsenasv pslgpmptaa qpsttgirkq wheditqdlr nhlvhklvqa ifptpdpaal |
| 601 | kdrrmenlva yarkvegdmy esannraeyy hllaekiyki qkeleekrrt rlqkqnmlpn |
| 661 | aagmvpvsmn pgpnmgqpqp gmtsslnqfg qmsmaqppiv prqtpplqhh gqlaqpgaln |
| 721 | ppmgygprmq qpsnqgqflp qtqfpsqgmn vtniplapss gqapvsqaqm sssscpvnsp |
| 781 | imppgsqgsh ihcpqlpqpa lhqnspspvp srtptphhtp psigaqqppa ttipapvptp |
| 841 | pamppgpqsq alhppprqtp tppttqlpqq vqpslpaaps adqpqqqprs qqstaasvpt |
| 901 | ptapllppqp atplsqpavs iegqvsnpps tsstevnsqa iaekqpsqev kmeakmevdq |
| 961 | pepadtqped iseskvedck mesteteers telkteikee edqpstsatq sspapgqskk |
| 1021 | kifkpeelrq almptlealy rqdpeslpfr qpvdpqllgi pdyfdivksp mdlstikrkl |
| 1081 | dtgqyqepwq yvddiwlmfn nawlynrkts rvykycskls evfeqeidpv mqslgyccgr |
| 1141 | klefspqtlc cygkqlctip rdatyysyqn ryhfcekcfn eiqgesvslg ddpsqpqtti |
| 1201 | nkeqfskrkn dtldpelfve ctecgrkmhq icvlhheiiw pagfvcdgcl kksartrken |
| 1261 | kfsakrlpst rlgtflenrv ndflrrqnhp esgevtvrvv hasdktvevk pgmkarfvds |
| 1321 | gemaesfpyr tkalfafeei dgvdlcffgm hvqeygsdcp ppnqrrvyis yldsvhffrp |
| 1381 | kclrtavyhe iligyleyvk klgyttghiw acppsegddy ifhchppdqk ipkpkrlqew |
| 1441 | ykkmldkavs erivhdykdi fkqatedrlt sakelpyfeg dfwpnvlees ikeleqeeee |
| 1501 | rkreentsne stdvtkgdsk nakkknnkkt sknksslsrg nkkkpgmpnv sndlsqklya |
| 1561 | tmekhkevff virliagpaa nslppivdpd plipcdlmdg rdafltlard khlefsslrr |
| 1621 | aqwstmcmlv elhtqsqdrf vytcneckhh vetrwhctvc edydlcitcy ntknhdhkme |
| 1681 | klglglddes nnqqaaatqs pgdsrrlsiq rciqslvhac qernancslp scqkmkrvvq |
| 1741 | htkgckrktn ggcpickqli alccyhakhc qenkcpvpfc lnikqklrqq qlqhrlqqaq |
| 1801 | mlrrrmasmq rtgvvgqqqg lpsptpatpt tptgqqpttp qtpqptsqpq ptppnsmppy |
| 1861 | lprtqaagpv sqgkaagqvt pptppqtaqp plpgpppaav emamqiqraa etqrqmahvq |
| 1921 | ifqrpiqhqm ppmtpmapmg mnpppmtrgp sghlepgmgp tgmqqqppws qgglpqpqql |
| 1981 | qsgmprpamm svaqhgqpln mapqpglgqv gisplkpgtv sqqalqnllr tlrspssplq |
| 2041 | qqqvlsilha npqllaafik qraakyansn pqpipgqpgm pqgqpglqpp tmpgqqgvhs |
| 2101 | npamqnmnpm qagvqraglp qqqpqqqlqp pmggmspqaq qmnmnhntmp sqfrdilrrq |
| 2161 | qmmqqqqqqg agpgigpgma nhnqfqqpqg vgyppqqqqr mqhhmqqmqq gnmgqigqlp |
| 2221 | qalgaeagas lqayqqrllq qqmgspvqpn pmspqqhmlp ngaqsphlqg qqipnslsnq |
| 2281 | vrspqpvpsp rpqsqpphss psprmqpqps phhvspqtss phpglvaaga npmeqghfas |
| 2341 | pdqnsmlsql asnpgmanlh gasatdlgls tdnsdlnsnl sqstldih |
| An exemplary EP300 nucleic acid sequence is provided at NCBI Accession | |
| No. NM_001362843, version NM_001362843.2, as set forth below (SEQ ID NO: 2): | |
| 1 | gagaaggagg aggacagcgc cgaggaggaa gaggttgatg gcggcggcgg agctccgaga |
| 61 | gacctcggct gggcaggggc cggccgtggc gggccgggga ctgcgcctct agagccgcga |
| 121 | gttctcggga attcgccgca gcggacgcgc tcggcgaatt tgtgctcttg tgccctcctc |
| 181 | cgggcttggg cccaggcccg gcccctcgca cttgccctta ccttttctat cgagtccgca |
| 241 | tccctctcca gccactgcga cccggcgaag agaaaaagga acttccccca ccccctcggg |
| 301 | tgccgtcgga gccccccagc ccacccctgg gtgcggcgcg gggaccccgg gccgaagaag |
| 361 | agatttcctg aggattctgg ttttcctcgc ttgtatctcc gaaagaatta aaaatggccg |
| 421 | agaatgtggt ggaaccgggg ccgccttcag ccaagcggcc taaactctca tctccggccc |
| 481 | tctcggcgtc cgccagcgat ggcacagatt ttggctctct atttgacttg gagcacgact |
| 541 | taccagatga attaatcaac tctacagaat tgggactaac caatggtggt gatattaatc |
| 601 | agcttcagac aagtcttggc atggtacaag atgcagcttc taaacataaa cagctgtcag |
| 661 | aattgctgcg atctggtagt tcccctaacc tcaatatggg agttggtggc ccaggtcaag |
| 721 | tcatggccag ccaggcccaa cagagcagtc ctggattagg tttgataaat agcatggtca |
| 781 | aaagcccaat gacacaggca ggcttgactt ctcccaacat ggggatgggc actagtggac |
| 841 | caaatcaggg tcctacgcag tcaacaggta tgatgaacag tccagtaaat cagcctgcca |
| 901 | tgggaatgaa cacagggatg aatgcgggca tgaatcctgg aatgttggct gcaggcaatg |
| 961 | gacaagggat aatgcctaat caagtcatga acggttcaat tggagcaggc cgagggcgac |
| 1021 | agaatatgca gtacccaaac ccaggcatgg gaagtgctgg caacttactg actgagcctc |
| 1081 | ttcagcaggg ctctccccag atgggaggac aaacaggatt gagaggcccc cagcctctta |
| 1141 | agatgggaat gatgaacaac cccaatcctt atggttcacc atatactcag aatcctggac |
| 1201 | agcagattgg agccagtggc cttggtctcc agattcagac aaaaactgta ctatcaaata |
| 1261 | acttatctcc atttgctatg gacaaaaagg cagttcctgg tggaggaatg cccaacatgg |
| 1321 | gtcaacagcc agccccgcag gtccagcagc caggcctggt gactccagtt gcccaaggga |
| 1381 | tgggttctgg agcacataca gctgatccag agaagcgcaa gctcatccag cagcagcttg |
| 1441 | ttctcctttt gcatgctcac aagtgccagc gccgggaaca ggccaatggg gaagtgaggc |
| 1501 | agtgcaacct tccccactgt cgcacaatga agaatgtcct aaaccacatg acacactgcc |
| 1561 | agtcaggcaa gtcttgccaa gtggcacact gtgcatcttc tcgacaaatc atttcacact |
| 1621 | ggaagaattg tacaagacat gattgtcctg tgtgtctccc cctcaaaaat gctggtgata |
| 1681 | agagaaatca acagccaatt ttgactggag cacccgttgg acttggaaat cctagctctc |
| 1741 | taggggtggg tcaacagtct gcccccaacc taagcactgt tagtcagatt gatcccagct |
| 1801 | ccatagaaag agcctatgca gctcttggac taccctatca agtaaatcag atgccgacac |
| 1861 | aaccccaggt gcaagcaaag aaccagcaga atcagcagcc tgggcagtct ccccaaggca |
| 1921 | tgcggcccat gagcaacatg agtgctagtc ctatgggagt aaatggaggt gtaggagttc |
| 1981 | aaacgccgag tcttctttct gactcaatgt tgcattcagc cataaattct caaaacccaa |
| 2041 | tgatgagtga aaatgccagt gtgccctccc tgggtcctat gccaacagca gctcaaccat |
| 2101 | ccactactgg aattcggaaa cagtggcacg aagatattac tcaggatctt cgaaatcatc |
| 2161 | ttgttcacaa actcgtccaa gccatatttc ctacgccgga tcctgctgct ttaaaagaca |
| 2221 | gacggatgga aaacctagtt gcatatgctc ggaaagttga aggggacatg tatgaatctg |
| 2281 | caaacaatcg agcggaatac taccaccttc tagctgagaa aatctataag atccagaaag |
| 2341 | aactagaaga aaaacgaagg accagactac agaagcagaa catgctacca aatgctgcag |
| 2401 | gcatggttcc agtttccatg aatccagggc ctaacatggg acagccgcaa ccaggaatga |
| 2461 | cttctagttt gaatcaattt ggccagatga gcatggccca gccccctatt gtaccccggc |
| 2521 | aaacccctcc tcttcagcac catggacagt tggctcaacc tggagctctc aacccgccta |
| 2581 | tgggctatgg gcctcgtatg caacagcctt ccaaccaggg ccagttcctt cctcagactc |
| 2641 | agttcccatc acagggaatg aatgtaacaa atatcccttt ggctccgtcc agcggtcaag |
| 2701 | ctccagtgtc tcaagcacaa atgtctagtt cttcctgccc ggtgaactct cctataatgc |
| 2761 | ctccagggtc tcaggggagc cacattcact gtccccagct tcctcaacca gctcttcatc |
| 2821 | agaattcacc ctcgcctgta cctagtcgta cccccacccc tcaccatact cccccaagca |
| 2881 | taggggctca gcagccacca gcaacaacaa ttccagcccc tgttcctaca cctcctgcca |
| 2941 | tgccacctgg gccacagtcc caggctctac atccccctcc aaggcagaca cctacaccac |
| 3001 | caacaacaca acttccccaa caagtgcagc cttcacttcc tgctgcacct tctgctgacc |
| 3061 | agccccagca gcagcctcgc tcacagcaga gcacagcagc gtctgttcct accccaacag |
| 3121 | caccgctgct tcctccgcag cctgcaactc cactttccca gccagctgta agcattgaag |
| 3181 | gacaggtatc aaatcctcca tctactagta gcacagaagt gaattctcag gccattgctg |
| 3241 | agaagcagcc ttcccaggaa gtgaagatgg aggccaaaat ggaagtggat caaccagaac |
| 3301 | cagcagatac tcagccggag gatatttcag agtctaaagt ggaagactgt aaaatggaat |
| 3361 | ctaccgaaac agaagagaga agcactgagt taaaaactga aataaaagag gaggaagacc |
| 3421 | agccaagtac ttcagctacc cagtcatctc cggctccagg acagtcaaag aaaaagattt |
| 3481 | tcaaaccaga agaactacga caggcactga tgccaacttt ggaggcactt taccgtcagg |
| 3541 | atccagaatc ccttcccttt cgtcaacctg tggaccctca gcttttagga atccctgatt |
| 3601 | actttgatat tgtgaagagc cccatggatc tttctaccat taagaggaag ttagacactg |
| 3661 | gacagtatca ggagccctgg cagtatgtcg atgatatttg gcttatgttc aataatgcct |
| 3721 | ggttatataa ccggaaaaca tcacgggtat acaaatactg ctccaagctc tctgaggtct |
| 3781 | ttgaacaaga aattgaccca gtgatgcaaa gccttggata ctgttgtggc agaaagttgg |
| 3841 | agttctctcc acagacactg tgttgctacg gcaaacagtt gtgcacaata cctcgtgatg |
| 3901 | ccacttatta cagttaccag aacaggtatc atttctgtga gaagtgtttc aatgagatcc |
| 3961 | aaggggagag cgtttctttg ggggatgacc cttcccagcc tcaaactaca ataaataaag |
| 4021 | aacaattttc caagagaaaa aatgacacac tggatcctga actgtttgtt gaatgtacag |
| 4081 | agtgcggaag aaagatgcat cagatctgtg tccttcacca tgagatcatc tggcctgctg |
| 4141 | gattcgtctg tgatggctgt ttaaagaaaa gtgcacgaac taggaaagaa aataagtttt |
| 4201 | ctgctaaaag gttgccatct accagacttg gcacctttct agagaatcgt gtgaatgact |
| 4261 | ttctgaggcg acagaatcac cctgagtcag gagaggtcac tgttagagta gttcatgctt |
| 4321 | ctgacaaaac cgtggaagta aaaccaggca tgaaagcaag gtttgtggac agtggagaga |
| 4381 | tggcagaatc ctttccatac cgaaccaaag ccctctttgc ctttgaagaa attgatggtg |
| 4441 | ttgacctgtg cttctttggc atgcatgttc aagagtatgg ctctgactgc cctccaccca |
| 4501 | accagaggag agtatacata tcttacctcg atagtgttca tttcttccgt cctaaatgct |
| 4561 | tgaggactgc agtctatcat gaaatcctaa ttggatattt agaatatgtc aagaaattag |
| 4621 | gttacacaac agggcatatt tgggcatgtc caccaagtga gggagatgat tatatcttcc |
| 4681 | attgccatcc tcctgaccag aagataccca agcccaagcg actgcaggaa tggtacaaaa |
| 4741 | aaatgcttga caaggctgta tcagagcgta ttgtccatga ctacaaggat atttttaaac |
| 4801 | aagctactga agatagatta acaagtgcaa aggaattgcc ttatttcgag ggtgatttct |
| 4861 | ggcccaatgt tctggaagaa agcattaagg aactggaaca ggaggaagaa gagagaaaac |
| 4921 | gagaggaaaa caccagcaat gaaagcacag atgtgaccaa gggagacagc aaaaatgcta |
| 4981 | aaaagaagaa taataagaaa accagcaaaa ataagagcag cctgagtagg ggcaacaaga |
| 5041 | agaaacccgg gatgcccaat gtatctaacg acctctcaca gaaactatat gccaccatgg |
| 5101 | agaagcataa agaggtcttc tttgtgatcc gcctcattgc tggccctgct gccaactccc |
| 5161 | tgcctcccat tgttgatcct gatcctctca tcccctgcga tctgatggat ggtcgggatg |
| 5221 | cgtttctcac gctggcaagg gacaagcacc tggagttctc ttcactccga agagcccagt |
| 5281 | ggtccaccat gtgcatgctg gtggagctgc acacgcagag ccaggaccgc tttgtctaca |
| 5341 | cctgcaatga atgcaagcac catgtggaga cacgctggca ctgtactgtc tgtgaggatt |
| 5401 | atgacttgtg tatcacctgc tataacacta aaaaccatga ccacaaaatg gagaaactag |
| 5461 | gccttggctt agatgatgag agcaacaacc agcaggctgc agccacccag agcccaggcg |
| 5521 | attctcgccg cctgagtatc cagcgctgca tccagtctct ggtccatgct tgccagtgtc |
| 5581 | ggaatgccaa ttgctcactg ccatcctgcc agaagatgaa gcgggttgtg cagcatacca |
| 5641 | agggttgcaa acggaaaacc aatggcgggt gccccatctg caagcagctc attgccctct |
| 5701 | gctgctacca tgccaagcac tgccaggaga acaaatgccc ggtgccgttc tgcctaaaca |
| 5761 | tcaagcagaa gctccggcag caacagctgc agcaccgact acagcaggcc caaatgcttc |
| 5821 | gcaggaggat ggccagcatg cagcggactg gtgtggttgg gcagcaacag ggcctccctt |
| 5881 | cccccactcc tgccactcca acgacaccaa ctggccaaca gccaaccacc ccgcagacgc |
| 5941 | cccagcccac ttctcagcct cagcctaccc ctcccaatag catgccaccc tacttgccca |
| 6001 | ggactcaagc tgctggccct gtgtcccagg gtaaggcagc aggccaggtg acccctccaa |
| 6061 | cccctcctca gactgctcag ccaccccttc cagggccccc acctgcagca gtggaaatgg |
| 6121 | caatgcagat tcagagagca gcggagacgc agcgccagat ggcccacgtg caaatttttc |
| 6181 | aaaggccaat ccaacaccag atgcccccga tgactcccat ggcccccatg ggtatgaacc |
| 6241 | cacctcccat gaccagaggt cccagtgggc atttggagcc agggatggga ccgacaggga |
| 6301 | tgcagcaaca gccaccctgg agccaaggag gattgcctca gccccagcaa ctacagtctg |
| 6361 | ggatgccaag gccagccatg atgtcagtgg cccagcatgg tcaacctttg aacatggctc |
| 6421 | cacaaccagg attgggccag gtaggtatca gcccactcaa accaggcact gtgtctcaac |
| 6481 | aagccttaca aaaccttttg cggactctca ggtctcccag ctctcccctg cagcagcaac |
| 6541 | aggtgcttag tatccttcac gccaaccccc agctgttggc tgcattcatc aagcagcggg |
| 6601 | ctgccaagta tgccaactct aatccacaac ccatccctgg gcagcctggc atgccccagg |
| 6661 | ggcagccagg gctacagcca cctaccatgc caggtcagca gggggtccac tccaatccag |
| 6721 | ccatgcagaa catgaatcca atgcaggcgg gcgttcagag ggctggcctg ccccagcagc |
| 6781 | aaccacagca gcaactccag ccacccatgg gagggatgag cccccaggct cagcagatga |
| 6841 | acatgaacca caacaccatg ccttcacaat tccgagacat cttgagacga cagcaaatga |
| 6901 | tgcaacagca gcagcaacag ggagcagggc caggaatagg ccctggaatg gccaaccata |
| 6961 | accagttcca gcaaccccaa ggagttggct acccaccaca gcagcagcag cggatgcagc |
| 7021 | atcacatgca acagatgcaa caaggaaata tgggacagat aggccagctt ccccaggcct |
| 7081 | tgggagcaga ggcaggtgcc agtctacagg cctatcagca gcgactcctt cagcaacaga |
| 7141 | tggggtcccc tgttcagccc aaccccatga gcccccagca gcatatgctc ccaaatcagg |
| 7201 | cccagtcccc acacctacaa ggccagcaga tccctaattc tctctccaat caagtgcgct |
| 7261 | ctccccagcc tgtcccttct ccacggccac agtcccagcc cccccactcc agtccttccc |
| 7321 | caaggatgca gcctcagcct tctccacacc acgtttcccc acagacaagt tccccacatc |
| 7381 | ctggactggt agctgcccag gccaacccca tggaacaagg gcattttgcc agcccggacc |
| 7441 | agaattcaat gctttctcag cttgctagca atccaggcat ggcaaacctc catggtgcaa |
| 7501 | gcgccacgga cctgggactc agcaccgata actcagactt gaattcaaac ctctcacaga |
| 7561 | gtacactaga catacactag agacaccttg tagtattttg ggagcaaaaa aattattttc |
| 7621 | tcttaacaag actttttgta ctgaaaacaa tttttttgaa tctttcgtag cctaaaagac |
| 7681 | aattttcctt ggaacacata agaactgtgc agtagccgtt tgtggtttaa agcaaacatg |
| 7741 | caagatgaac ctgagggatg atagaataca aagaatatat ttttgttatg gctggttacc |
| 7801 | accagccttt cttccccttt gtgtgtgtgg ttcaagtgtg cactgggagg aggctgaggc |
| 7861 | ctgtgaagcc aaacaatatg ctcctgcctt gcacctccaa taggttttat tatttttttt |
| 7921 | aaattaatga acatatgtaa tattaatagt tattatttac tggtgcagat ggttgacatt |
| 7981 | tttccctatt ttcctcactt tatggaagag ttaaaacatt tctaaaccag aggacaaaag |
| 8041 | gggttaatgt tactttaaaa ttacattcta tatatatata aatatatata aatatatatt |
| 8101 | aaaataccag ttttttttct ctgggtgcaa agatgttcat tcttttaaaa aatgtttaaa |
| 8161 | aaaaaaaaaa aactgccttt cttcccctca agtcaacttt tgtgctccag aaaattttct |
| 8221 | attctgtaag tctgagcgta aaacttcaag tattaaaata atttgtacat gtagagagaa |
| 8281 | aaatgacttt ttcaaaaata tacaggggca gctgccaaat tgatgtatta tatattgtgg |
| 8341 | tttctgtttc ttgaaagaat ttttttcgtt atttttacat ctaacaaagt aaaaaaatta |
| 8401 | aaaagagggt aagaaacgat tccggtggga tgattttaac atgcaaaatg tccctggggg |
| 8461 | tttcttcttt gcttgctttc ttcctcctta ccctaccccc cactcacaca cacacacaca |
| 8521 | cacacacaca cacacacaca cacacacttt ctataaaact tgaaaatagc aaaaaccctc |
| 8581 | aactgttgta aatcatgcaa ttaaagttga ttacttataa atatgaactt tggatcactg |
| 8641 | tatagactgt taaatttgat ttcttattac ctattgttaa ataaactgtg tgagacagac |
| 8701 | a |
| An exemplary CBP amino acid sequence is provided at NCBI Accession No. | |
| XP_011520683, version XP_011520683.1, as set forth below (SEQ ID NO: 3): | |
| 1 | mlsypewtcw rgshqmgitg ntspfgqpfs qaggqpmgat gvnpqlaskq smvnslptfp |
| 61 | tdikntsvtn vpnmsqmqts vgivptqaia tgptadpekr kliqqqlvll lhahkcqrre |
| 121 | qangevracs lphcrtmknv Inhmthcqag kacqvahcas srqiishwkn ctrhdcpvcl |
| 181 | plknasdkrn qqtilgspas giqntigsvg tgqqnatsls npnpidpssm qrayaalglp |
| 241 | ymnqpqtqlq pqvpgqqpaq pqthqqmrtl nplgnnpmni paggittdqq ppnlisesal |
| 301 | ptslgatnpl mndgsnsgni gtlstiptaa ppsstgvrkg whehvtqdlr shlvhklvqa |
| 361 | ifptpdpaal kdrrmenlva yakkvegdmy esansrdeyy hllaekiyki qkeleekrrs |
| 421 | rlhkqgilgn qpalpapgaq ppvipqaqpv rppngplslp vnrmqvsqgm nsfnpmslgn |
| 481 | vqlpqapmgp raaspmnhsv qmnsmgsvpg maispsrmpq ppnmmgahtn nmmagapaqs |
| 541 | qflpqnqfps ssgamsvgmg qppaqtgvsq gqvpgaalpn plnmlgpqas qlpcppvtqs |
| 601 | plhptpppas taagmpslqh ttppgmtppq paaptqpstp vsssgqtptp tpgsvpsatq |
| 661 | tqstptvqaa aqaqvtpqpq tpvqppsvat pqssqqqptp vhaqppgtpl sqaaasidnr |
| 721 | vptpssvasa etnsqqpgpd vpvlemktet qaedtepdpg eskgeprsem meedlqgasq |
| 781 | vkeetdiaeq ksepmevdek kpevkvevke eeesssngta sqstspsqpr kkifkpeelr |
| 841 | qalmptleal yrqdpeslpf rqpvdpqllg ipdyfdivkn pmdlstikrk ldtgqyqepw |
| 901 | qyvddvwlmf nnawlynrkt srvykfcskl aevfeqeidp vmqslgyccg rkyefspqtl |
| 961 | ccygkqlcti prdaayysyq nryhfcekcf teiqgenvtl gddpsqpqtt iskdqfekkk |
| 1021 | ndtldpepfv dckecgrkmh qicvlhydii wpsgfvcdnc lkktgrprke nkfsakrlqt |
| 1081 | trlgnhledr vnkflrrqnh peagevfvrv vassdktvev kpgmksrfvd sgemsesfpy |
| 1141 | rtkalfafee idgvdvcffg mhvqeygsdc pppntrrvyi syldsihffr prclrtavyh |
| 1201 | eiligyleyv kklgyvtghi wacppsegdd yifhchppdq kipkpkrlqe wykkmldkaf |
| 1261 | aeriihdykd ifkqatedrl tsakelpyfe gdfwpnvlee sikeleqeee erkkeestaa |
| 1321 | settegsqgd sknakkknnk ktnknkssis rankkkpsmp nvsndlsqkl yatmekhkev |
| 1381 | ffvihlhagp vintlppivd pdpllscdlm dgrdafltla rdkhwefssl rrskwstlcm |
| 1441 | lvelhtqgqd rfvytcneck hhvetrwhct vcedydlcin cyntkshahk mvkwglgldd |
| 1501 | egssqgepqs kspqesrils iqrciqslvh acqcrnancs Ipscqkmkrv vqhtkgckrk |
| 1561 | tnggcpvckq lialccyhak hcqenkcpvp felnikhklr qqqiqhrlqq aqlmrrrmat |
| 1621 | mntrnvpqqs lpsptsappg tptqqpstpq tpqppaqpqp spvsmspagf psvartqppt |
| 1681 | tvstgkptsq vpappppaqp ppaaveaarq iereaqqqqh lyrvninnsm ppgrtgmgtp |
| 1741 | gsqmapvsln vprpnqvsgp vmpsmppgqw qqaplpqqqp mpglprpvis mqaqaavagp |
| 1801 | rmpsvqpprs ispsalqdll rtlkspsspq qqqqvlnilk snpqlmaafi kqrtakyvan |
| 1861 | qpgmqpqpgl qsqpgmqpqp gmhqqpslan Inamqagvpr pgvppqqqam gglnpqgqal |
| 1921 | nimnpghnpn masmnpqyre mlrrqllqqq qqqqqqqqqq qqqqqgsagm aggmaghgqf |
| 1981 | qqpqgpggyp pamqqqqrmq qhlplqgssm gqmaaqmgql gqmgqpglga dstpniqqal |
| 2041 | qqrilqqqqm kqqigspgqp npmspqqhml sgqpqashlp gqqiatslsn qvrspapvqs |
| 2101 | prpqsqpphs spspriqpqp sphhvspqtg sphpglavtm assidqghlg npeqsamlpq |
| 2161 | Intpsrsals selslvgdtt gdtlekfveg l |
| An exemplary CBP nucleic acid sequence is provided at NCBI Accession No. | |
| XM_011522381, version XM_011522381.2, as set forth below (SEQ ID NO: 4): | |
| 1 | cagatgacag ttgaaggaag cttcttgcaa atcagaaatg tgcttaatat ttatcgagct |
| 61 | accatcttgc ctagattaag tcatttgaac tcgaaattga gtctggtttg tggacttgca |
| 121 | gaagaattag tgtctcagtt cacttaagta gagcacctag tggtgacaag aatgtgattg |
| 181 | ctttccaaaa ggtgagaaat gtcacctagg aggactacat ggggaaggaa atcacctgcg |
| 241 | tatgaaatgc acagagctag aactttctgg ttatactcct ttggttttta tttgtgtttc |
| 301 | tgtacaggca tttcagcaga aagggccagt tgtgttgagt gttgatttgt ttgtagccta |
| 361 | gacttttaga gctgaaagaa ataatacgat ccatcttgtt caagacactc atcttacagg |
| 421 | cgaggagttt gaagtccata gaaggaatgt taagttaccc agagtggaca tgctggcgtg |
| 481 | gcagtcacca gatgggaata actgggaaca caagtccatt tggacagccc tttagtcaag |
| 541 | ctggagggca gccaatggga gccactggag tgaaccccca gttagccagc aaacagagca |
| 601 | tggtcaacag tttgcccacc ttccctacag atatcaagaa tacttcagtc accaacgtgc |
| 661 | caaatatgtc tcagatgcaa acatcagtgg gaattgtacc cacacaagca attgcaacag |
| 721 | gccccactgc agatcctgaa aaacgcaaac tgatacagca gcagctggtt ctactgcttc |
| 781 | atgctcataa gtgtcagaga cgagagcaag caaacggaga ggttcgggcc tgctcgctcc |
| 841 | cgcattgtcg aaccatgaaa aacgttttga atcacatgac gcattgtcag gctgggaaag |
| 901 | cctgccaagt tgcccattgt gcatcttcac gacaaatcat ctctcattgg aagaactgca |
| 961 | cacgacatga ctgtcctgtt tgcctccctt tgaaaaatgc cagtgacaag cgaaaccaac |
| 1021 | aaaccatcct ggggtctcca gctagtggaa ttcaaaacac aattggttct gttggcacag |
| 1081 | ggcaacagaa tgccacttct ttaagtaacc caaatcccat agaccccagc tccatgcagc |
| 1141 | gagcctatgc tgctctcgga ctcccctaca tgaaccagcc ccagacgcag ctgcagcctc |
| 1201 | aggttcctgg ccagcaacca gcacagcctc aaacccacca gcagatgagg actctcaacc |
| 1261 | ccctgggaaa taatccaatg aacattccag caggaggaat aacaacagat cagcagcccc |
| 1321 | caaacttgat ttcagaatca gctcttccga cttccctggg ggccacaaac ccactgatga |
| 1381 | acgatggctc caactctggt aacattggaa ccctcagcac tataccaaca gcagctcctc |
| 1441 | cttctagcac cggtgtaagg aaaggctggc acgaacatgt cactcaggac ctgcggagcc |
| 1501 | atctagtgca taaactcgtc caagccatct tcccaacacc tgatcccgca gctctaaagg |
| 1561 | atcgccgcat ggaaaacctg gtagcctatg ctaagaaagt ggaaggggac atgtacgagt |
| 1621 | ctgccaacag cagggatgaa tattatcact tattagcaga gaaaatctac aagatacaaa |
| 1681 | aagaactaga agaaaaacgg aggtcgcgtt tacataaaca aggcatcttg gggaaccagc |
| 1741 | cagccttacc agccccgggg gctcagcccc ctgtgattcc acaggcacaa cctgtgagac |
| 1801 | ctccaaatgg acccctgtcc ctgccagtga atcgcatgca agtttctcaa gggatgaatt |
| 1861 | catttaaccc catgtccttg gggaacgtcc agttgccaca agcacccatg ggacctcgtg |
| 1921 | cagcctcccc aatgaaccac tctgtccaga tgaacagcat gggctcagtg ccagggatgg |
| 1981 | ccatttctcc ttcccgaatg cctcagcctc cgaacatgat gggtgcacac accaacaaca |
| 2041 | tgatggccca ggcgcccgct cagagccagt ttctgccaca gaaccagttc ccgtcatcca |
| 2101 | gcggggcgat gagtgtgggc atggggcagc cgccagccca aacaggcgtg tcacagggac |
| 2161 | aggtgcctgg tgctgctctt cctaaccctc tcaacatgct ggggcctcag gccagccagc |
| 2221 | taccttgccc tccagtgaca cagtcaccac tgcacccaac accgcctcct gcttccacgg |
| 2281 | ctgctggcat gccatctctc cagcacacga caccacctgg gatgactcct ccccagccag |
| 2341 | cagctcccac tcagccatca actcctgtgt cgtcttccgg gcagactccc accccgactc |
| 2401 | ctggctcagt gcccagtgct acccaaaccc agagcacccc tacagtccag gcagcagccc |
| 2461 | aggcccaggt gaccccgcag cctcaaaccc cagttcagcc cccgtctgtg gctacccctc |
| 2521 | agtcatcgca gcaacagccg acgcctgtgc acgcccagcc tcctggcaca ccgctttccc |
| 2581 | aggcagcagc cagcattgat aacagagtcc ctaccccctc ctcggtggcc agcgcagaaa |
| 2641 | ccaattccca gcagccagga cctgacgtac ctgtgctgga aatgaagacg gagacccaag |
| 2701 | cagaggacac tgagcccgat cctggtgaat ccaaagggga gcccaggtct gagatgatgg |
| 2761 | aggaggattt gcaaggagct tcccaagtta aagaagaaac agacatagca gagcagaaat |
| 2821 | cagaaccaat ggaagtggat gaaaagaaac ctgaagtgaa agtagaagtt aaagaggaag |
| 2881 | aagagagtag cagtaacggc acagcctctc agtcaacatc tccttcgcag ccgcgcaaaa |
| 2941 | aaatctttaa accagaggag ttacgccagg ccctcatgcc aaccctagaa gcactgtatc |
| 3001 | gacaggaccc agagtcatta cctttccggc agcctgtaga tccccagctc ctcggaattc |
| 3061 | cagactattt tgacatcgta aagaatccca tggacctctc caccatcaag cggaagctgg |
| 3121 | acacagggca ataccaagag ccctggcagt acgtggacga cgtctggctc atgttcaaca |
| 3181 | atgcctggct ctataatcgc aagacatccc gagtctataa gttttgcagt aagcttgcag |
| 3241 | aggtctttga gcaggaaatt gaccctgtca tgcagtccct tggatattgc tgtggacgca |
| 3301 | agtatgagtt ttccccacag actttgtgct gctatgggaa gcagctgtgt accattcctc |
| 3361 | gcgatgctgc ctactacagc tatcagaata ggtatcattt ctgtgagaag tgtttcacag |
| 3421 | agatccaggg cgagaatgtg accctgggtg acgacccttc acagccccag acgacaattt |
| 3481 | caaaggatca gtttgaaaag aagaaaaatg ataccttaga ccccgaacct ttcgttgatt |
| 3541 | gcaaggagtg tggccggaag atgcatcaga tttgcgttct gcactatgac atcatttggc |
| 3601 | cttcaggttt tgtgtgcgac aactgcttga agaaaactgg cagacctcga aaagaaaaca |
| 3661 | aattcagtgc taagaggctg cagaccacaa gactgggaaa ccacttggaa gaccgagtga |
| 3721 | acaaattttt gcggcgccag aatcaccctg aagccgggga ggtttttgtc cgagtggtgg |
| 3781 | ccagctcaga caagacggtg gaggtcaagc ccgggatgaa gtcacggttt gtggattctg |
| 3841 | gggaaatgtc tgaatctttc ccatatcgaa ccaaagctct gtttgctttt gaggaaattg |
| 3901 | acggcgtgga tgtctgcttt tttggaatgc acgtccaaga atacggctct gattgccccc |
| 3961 | ctccaaacac gaggcgtgtg tacatttctt atctggatag tattcatttc ttccggccac |
| 4021 | gttgcctccg cacagccgtt taccatgaga tccttattgg atatttagag tatgtgaaga |
| 4081 | aattagggta tgtgacaggg cacatctggg cctgtcctcc aagtgaagga gatgattaca |
| 4141 | tcttccattg ccacccacct gatcaaaaaa tacccaagcc aaaacgactg caggagtggt |
| 4201 | acaaaaagat gctggacaag gcgtttgcag agcggatcat ccatgactac aaggatattt |
| 4261 | tcaaacaagc aactgaagac aggctcacca gtgccaagga actgccctat tttgaaggtg |
| 4321 | atttctggcc caatgtgtta gaagagagca ttaaggaact agaacaagaa gaagaggaga |
| 4381 | ggaaaaagga agagagcact gcagccagtg aaaccactga gggcagtcag ggcgacagca |
| 4441 | agaatgccaa gaagaagaac aacaagaaaa ccaacaagaa caaaagcagc atcagccgcg |
| 4501 | ccaacaagaa gaagcccagc atgcccaacg tgtccaatga cctgtcccag aagctgtatg |
| 4561 | ccaccatgga gaagcacaag gaggtcttct tcgtgatcca cctgcacgct gggcctgtca |
| 4621 | tcaacaccct gccccccatc gtcgaccccg accccctgct cagctgtgac ctcatggatg |
| 4681 | ggcgcgacgc cttcctcacc ctcgccagag acaagcactg ggagttctcc tccttgcgcc |
| 4741 | gctccaagtg gtccacgctc tgcatgctgg tggagctgca cacccagggc caggaccgct |
| 4801 | ttgtctacac ctgcaacgag tgcaagcacc acgtggagac gcgctggcac tgcactgtgt |
| 4861 | gcgaggacta cgacctctgc atcaactgct ataacacgaa gagccatgcc cataagatgg |
| 4921 | tgaagtgggg gctgggcctg gatgacgagg gcagcagcca gggcgagcca cagtcaaaga |
| 4981 | gcccccagga gtcacgccgg ctgagcatcc agcgctgcat ccagtcgctg gtgcacgcgt |
| 5041 | gccagtgccg caacgccaac tgctcgctgc catcctgcca gaagatgaag cgggtggtgc |
| 5101 | agcacaccaa gggctgcaaa cgcaagacca acgggggctg cccggtgtgc aagcagctca |
| 5161 | tcgccctctg ctgctaccac gccaagcact gccaagaaaa caaatgcccc gtgcccttct |
| 5221 | gcctcaacat caaacacaag ctccgccagc agcagatcca gcaccgcctg cagcaggccc |
| 5281 | agctcatgcg ccggcggatg gccaccatga acacccgcaa cgtgcctcag cagagtctgc |
| 5341 | cttctcctac ctcagcaccg cccgggaccc ccacacagca gcccagcaca ccccagacgc |
| 5401 | cgcagccccc tgcccagccc caaccctcac ccgtgagcat gtcaccagct ggcttcccca |
| 5461 | gcgtggcccg gactcagccc cccaccacgg tgtccacagg gaagcctacc agccaggtgc |
| 5521 | cggccccccc acccccggcc cagccccctc ctgcagcggt ggaagcggct cggcagatcg |
| 5581 | agcgtgaggc ccagcagcag cagcacctgt accgggtgaa catcaacaac agcatgcccc |
| 5641 | caggacgcac gggcatgggg accccgggga gccagatggc ccccgtgagc ctgaatgtgc |
| 5701 | cccgacccaa ccaggtgagc gggcccgtca tgcccagcat gcctcccggg cagtggcagc |
| 5761 | aggcgcccct tccccagcag cagcccatgc caggcttgcc caggcctgtg atatccatgc |
| 5821 | aggcccaggc ggccgtggct gggccccgga tgcccagcgt gcagccaccc aggagcatct |
| 5881 | cacccagcgc tctgcaagac ctgctgcgga ccctgaagtc gcccagctcc cctcagcagc |
| 5941 | aacagcaggt gctgaacatt ctcaaatcaa acccgcagct aatggcagct ttcatcaaac |
| 6001 | agcgcacagc caagtacgtg gccaatcagc ccggcatgca gccccagcct ggcctccagt |
| 6061 | cccagcccgg catgcaaccc cagcctggca tgcaccagca gcccagcctg cagaacctga |
| 6121 | atgccatgca ggctggcgtg ccgcggcccg gtgtgcctcc acagcagcag gcgatgggag |
| 6181 | gcctgaaccc ccagggccag gccttgaaca tcatgaaccc aggacacaac cccaacatgg |
| 6241 | cgagtatgaa tccacagtac cgagaaatgt tacggaggca gctgctgcag cagcagcagc |
| 6301 | aacagcagca gcaacaacag cagcaacagc agcagcagca agggagtgcc ggcatggctg |
| 6361 | ggggcatggc ggggcacggc cagttccagc agcctcaagg acccggaggc tacccaccgg |
| 6421 | ccatgcagca gcagcagcgc atgcagcagc atctccccct ccagggcagc tccatgggcc |
| 6481 | agatggcggc tcagatggga cagcttggcc agatggggca gccggggctg ggggcagaca |
| 6541 | gcacccccaa catccagcaa gccctgcagc agcggattct gcagcaacag cagatgaagc |
| 6601 | agcagattgg gtccccaggc cagccgaacc ccatgagccc ccagcaacac atgctctcag |
| 6661 | gacagccaca ggcctcgcat ctccctggcc agcagatcgc cacgtccctt agtaaccagg |
| 6721 | tgcggtctcc agcccctgtc cagtctccac ggccccagtc ccagcctcca cattccagcc |
| 6781 | cgtcaccacg gatacagccc cagccttcgc cacaccacgt ctcaccccag actggttccc |
| 6841 | cccaccccgg actcgcagtc accatggcca gctccataga tcagggacac ttggggaacc |
| 6901 | ccgaacagag tgcaatgctc ccccagctga acacccccag caggagtgcg ctgtccagcg |
| 6961 | aactgtccct ggtcggggac accacggggg acacgctaga gaagtttgtg gagggcttgt |
| 7021 | agcattgtga gagcatcacc ttttcccttt catgttcttg gaccttttgt actgaaaatc |
| 7081 | caggcatcta ggttcttttt attcctagat ggaactgcga cttccgagcc atggaagggt |
| 7141 | ggattgatgt ttaaagaaac aatacaaaga atatattttt ttgttaaaaa ccagttgatt |
| 7201 | taaatatctg gtctctctct ttggtttttt tttggcgggg gggtgggggg ggttcttttt |
| 7261 | tttccgtttt gtttttgttt ggggggaggg gggttttgtt tggattcttt ttgtcgtcat |
| 7321 | tgctggtgac tcatgccttt ttttaacggg aaaaacaagt tcattatatt catatttttt |
| 7381 | atttgtattt tcaagacttt aaacatttat gtttaaaagt aagaagaaaa ataatattca |
| 7441 | gaactgattc ctgaaataat gcaagcttat aatgtatccc gataactttg tgatgtttcg |
| 7501 | ggaagatttt tttctatagt gaactctgtg ggcgtctccc agtattaccc tggatgatag |
| 7561 | gaattgactc cggcgtgcac acacgtacac acccacacac atctatctat acataatggc |
| 7621 | tgaagccaaa cttgtcttgc agatgtagaa attgttgctt tgtttctctg ataaaactgg |
| 7681 | ttttagacaa aaaataggga tgatcactct tagaccatgc taatgttact agagaagaag |
| 7741 | ccttcttttc tttcttctat gtgaaacttg aaatgaggaa aagcaattct agtgtaaatc |
| 7801 | atgcaagcgc tctaattcct ataaatacga aactcgagaa gattcaatca ctgtatagaa |
| 7861 | tggtaaaata ccaactcatt tcttatatca tattgttaaa taaactgtgt gcaacagaca |
| 7921 | aaaagggtgg tccttcttga attcatgtac atggtattaa cacttagtgt tcggggtttt |
| 7981 | ttgttatgaa aatgctgttt tcaacattgt atttggacta tgcatgtgtt ttttccccat |
| 8041 | tgtatataaa gtaccgctta aaattgatat aaattactga ggtttttaac atgtattctg |
| 8101 | ttctttaaga tccctgtaag aatgtttaag gtttttattt atttatatat attttttgag |
| 8161 | tctgttcttt gtaagacatg gttctggttg ttcgctcata gcggagaggc tggggctgcg |
| 8221 | gttgtggttg tggcggcgtg ggtggtggct gggaactgtg gcccaggctt agcggccgcc |
| 8281 | cggaggcttt tcttcccgga gactgaggtg ggcgactgag gtgggcggct cagcgttggc |
| 8341 | cccacacatt cgaggctcac aggtgattgt cgctcacaca gttagggtcg tcagttggtc |
| 8401 | tgaaactgca tttggcccac tcctccatcc tccctgtccg tcgtagctgc cacccccaga |
| 8461 | ggcggcgctt cttcccgtgt tcaggcggct cccccccccc gtacacgact cccagaatct |
| 8521 | gaggcagaga gtgctccagg ctcgcgaggt gctttctgac ttccccccaa atcctgccgc |
| 8581 | tgccgcgcag catgtcccgt gtggcgtttg aggaaatgct gagggacaga caccttggag |
| 8641 | caccagctcc ggtccctgtt acagtgagaa aggtccccca cttcggggga tacttgcact |
| 8701 | tagccacatg gtcctgcctc ccttggagtc cagttccagg ctcccttact gagtgggtga |
| 8761 | gacaagttca caaaaaccgt aaaactgaga ggaggaccat gggcagggga gctgaagttc |
| 8821 | atcccctaag tctaccaccc ccagcaccca gagaacccac tttatcccta gtcccccaac |
| 8881 | aaaggctggt ctaggtgggg gtgatggtaa ttttagaaat cacgccccaa atagcttccg |
| 8941 | tttgggccct tacattcaca gataggtttt aaatagctga atacttggtt tgggaatctg |
| 9001 | aattcgagga acctttctaa gaagttggaa aggtccgatc tagttttagc acagagcttt |
| 9061 | gaaccttgag ttataaaatg cagaataatt caagtaaaaa taagaccacc atctggcacc |
| 9121 | cctgaccagc ccccattcac cccatcccag gaggggaagc acaggccggg cctccggtgg |
| 9181 | agattgctgc cactgctcgg cctgctgggt tcttaacctc cagtgtcctc ttcatctttt |
| 9241 | ccacccgtag ggaaaccttg agccatgtgt tcaaacaaga agtggggcta gagcccgaga |
| 9301 | gcagcagctc taagcccaca ctcagaaagt ggcgccctcc tggttgtgca gccttttaat |
| 9361 | gtgggcagtg gaggggcctc tgtttcaggt tatcctggaa ttcaaaacgt tatgtaccaa |
| 9421 | cctcatcctc tttggagtct gcatcctgtg caaccgtctt gggcaatcca gatgtcgaag |
| 9481 | gatgtgaccg agagcatggt ctgtggatgc taaccctaag tttgtcgtaa ggaaatttct |
| 9541 | gtaagaaacc tggaaagccc caacgctgtg tctcatgctg tatacttaag aggagaagaa |
| 9601 | aaagtcctat atttgtgatc aaaaagagga aacttgaaat gtgatggtgt ttataataaa |
| 9661 | agatggtaaa acta |

### Cereblon (CRBN)

Human CRBN is a 442 amino acid E3 ubiquitin ligase with an apparent molecular weight of ˜51 kDa. CRBN contains the N-terminal part (237-amino acids from ammino acid 81 to 317) of ATP-dependent Lon protease domain without the conserved Walker A and Walker B motifs, 11 casein kinase II phosphorylation sites, 4 protein kinase C phosphorylation sites, 1 N-linked glycosylation site, and 2 myristoylation sites.

CRBN is widely expressed in testis, spleen, prostate, liver, pancreas, placenta, kidney, lung, skeletal muscle, ovary, small intestine, peripheral blood leukocytes, colon, brain, and retina, and is localized in the cytoplasm, nucleus, and plasma membrane (e.g., peripheral membrane). (Chang et al. Int. J. Biochem. Mol. Biol. 2:287-94 (2011)). Cereblon is an E3 ubiquitin ligase, and it forms complexes with damaged DNA binding protein 1 (DDB1), Cullin-4A (CUL4A), and regulator of cullins 1 (ROC1). This complex also ubiquitinates a number of other proteins. Cereblon ubiquitination of target proteins results in increased levels of fibroblast growth factor 8 (FGF8) and fibroblast growth factor 10 (FGF10). FGF8, in turn, regulates a number of developmental processes, such as limb and auditory vesicle formation.

| An exemplary CRBN amino acid sequence is provided at NCBI Accession No. | |
|---|---|
| XP_011532093, version XP_011532093.1, as set forth below (SEQ ID NO: 5): | |
| 1 | magegdqqda ahnmgnhlpl lpaeseeede mevedqdske akkpniinfd tslptshtyl |
| 61 | gadmeefhgr tlhdddscqv ipvlpqvmmi lipgqtlplq lfhpqevsmv rnliqkdrtf |
| 121 | avlaysnvqe reaqfgttae iyayreeqdf gieivkvkai grqrfkvlel rtqsdgiqqa |
| 181 | kvqilpecvl pstmsavqle slnkcqifps kpvsredqcs ykwwqkyqkr kfhcanltsw |
| 241 | prwlyslyda etlmdrikkq lrewdenlkd dslpsnpidf syrvaaclpi ddvlriqllk |
| 301 | igsaiqrlrc eldimnkcts lcckqcqete ittkneifry awtvaqckic ashigwkfta |
| 361 | tkkdmspqkf wgltrsallp tipdtedeis pdkvilcl |
| An exemplary CRBN nucleic acid sequence is provided at NCBI Accession No. | |
| XM_011533791, version XM_011533791.3, as set forth below (SEQ ID NO: 6): | |
| 1 | gcgggtaaac agacatggcc ggcgaaggag atcagcagga cgctgcgcac aacatgggca |
| 61 | accacctgcc gctcctgcct gcagagagtg aggaagaaga tgaaatggaa gttgaagacc |
| 121 | aggatagtaa agaagccaaa aaaccaaaca tcataaattt tgacaccagt ctgccgacat |
| 181 | cacatacata cctaggtgct gatatggaag aatttcatgg caggactttg cacgatgacg |
| 241 | acagctgtca ggtgattcca gttcttccac aagtgatgat gatcctgatt cccggacaga |
| 301 | cattacctct tcagcttttt caccctcaag aagtcagtat ggtgcggaat ttaattcaga |
| 361 | aagatagaac ctttgctgtt cttgcataca gcaatgtaca ggaaagggaa gcacagtttg |
| 421 | gaacaacagc agagatatat gcctatcgag aagaacagga ttttggaatt gagatagtga |
| 481 | aagtgaaagc aattggaaga caaaggttca aagtccttga gctaagaaca cagtcagatg |
| 541 | gaatccagca agctaaagtg caaattcttc ccgaatgtgt gttgccttca accatgtctg |
| 601 | cagttcaatt agaatccctc aataagtgcc agatatttcc ttcaaaacct gtctcaagag |
| 661 | aagaccaatg ttcatataaa tggtggcaga aataccagaa gagaaagttt cattgtgcaa |
| 721 | atctaacttc atggcctcgc tggctgtatt ccttatatga tgctgagacc ttaatggaca |
| 781 | gaatcaagaa acagctacgt gaatgggatg aaaatctaaa agatgattct cttccttcaa |
| 841 | atccaataga tttttcttac agagtagctg cttgtcttcc tattgatgat gtattgagaa |
| 901 | ttcagctcct taaaattggc agtgctatcc agcgacttcg ctgtgaatta gacattatga |
| 961 | ataaatgtac ttccctttgc tgtaaacaat gtcaagaaac agaaataaca accaaaaatg |
| 1021 | aaatattcag gtatgcctgg actgttgccc agtgtaagat ctgtgcaagc catattggat |
| 1081 | ggaagtttac ggccaccaaa aaagacatgt cacctcaaaa attttggggc ttaacgcgat |
| 1141 | ctgctctgtt gcccacgatc ccagacactg aagatgaaat aagtccagac aaagtaatac |
| 1201 | tttgcttgta aacagatgtg atagagataa agttatctaa caaattggtt atattctaag |
| 1261 | atctgctttg gaaattattg cctctgatac atacctaagt aaacataaca ttaataccta |
| 1321 | agtaaacata acattacttg gagggttgca gtttctaagt gaaactgtat ttgaaacttt |
| 1381 | taagtatact ttaggaaaca agcatgaacg gcagtctaga ataccagaaa catctacttg |
| 1441 | ggtagcttgg tgccattatc ctgtggaatc tgatatgtct ggtagcatgt cattgatggg |
| 1501 | acatgaagac atctttggaa atgatgagat tatttcctgt gttaaaaaaa aaaaaaatct |
| 1561 | taaattccta caatgtgaaa ctgaaactaa taatttgatc ctgatgtatg ggacagcgta |
| 1621 | tctgtaccag tgctctaaat aacaaaagct agggtgacaa gtacatgttc cttttggaaa |
| 1681 | gaagcaaggc aatgtatatt aattattcta aaagggcttt gttcctttcc attttcttta |
| 1741 | acttctctga gatactgatt tgtaaatttt gaaaattagt taaaatatgc agttttttga |
| 1801 | gcccacgaat agttgtcatt tcctttatgt gcctgttagt aaaaagtagt attgtgtatt |
| 1861 | tgctcagtat ctgaactata agcccattta tactgttcca tacaaaagct atttttcaaa |
| 1921 | aattaatttg aaccaaaact actactatag ggaaaagatg ccaaaacatg tcccctcacc |
| 1981 | cagactaaac ttgatactgt attattttgt tcaatgtaaa ttgaagaaaa tctgtaagta |
| 2041 | agtaaacctt aagtgtgaaa ctaaacatgt tctttgttca aataatgtaa aatatctact |
| 2101 | cataatttta aagttctaaa aaggctgctt tgccacctac ctctttgctt atattcaagg |
| 2161 | gtttagtggg tccttccttc cacatttgta tgataacttt cgttttattg gtagtccttt |
| 2221 | gctactttaa aactaatcag gttaaatgtt tactcaaatg tagtaatagt atgaggccaa |
| 2281 | aatacatgcc ttgaaggtgg agtgaaatca gttgagtgtc acactgcata tttatgtaaa |
| 2341 | tagagtaaat gaaaattcaa gaatacttcc aatcaggata tgcccctgcc ttattttttc |
| 2401 | taaggtctac ttttactact ttaataattt tgggtttgtg tgacttcact ggtctggttt |
| 2461 | gttcctatag ttcttacact gcactaaaac ttacttactc caggaagttc ctgtcagtag |
| 2521 | catagtacct ctctgtaggc tgaactctaa accagttgca atgagttacc ttgtccctct |
| 2581 | gaaaagttaa aatttcagta tgtcccatac cgtttactag caaataaaat tttctgatct |
| 2641 | gtaaaaacac acatatacta tactactact gaaaatggtt ttacacatga caaaaccaga |
| 2701 | cctgatagct aaatctgcat actttctata cagaaatagt atgtattata tgacgttggg |
| 2761 | tctaaatctg tattttggca gtacttaaat attttaagta agtatttcaa agtattattt |
| 2821 | tataatgcat ctaattactg acctgtatgc agtcatttct gaggctttct tgcatcatag |
| 2881 | cccctgtgac atttcctctt agaaatatta cactctacaa aattgtttta tcaaggtcca |
| 2941 | aaattactat ttgctcatag agtacaaaga tgttatgact ggctacacag aaaggaaata |
| 3001 | aattatgaaa tgtcagtata gcagctgaaa ttatagcagc tgaaataaac agtttgtatt |
| 3061 | atacatttta tttacctgat gaaagcattt aggactcaaa tctttagaga atatataata |
| 3121 | atagccatat ccttttaatt aataaaataa cactttaaag acaggcatat taccattgta |
| 3181 | tatgacacct aatacacatt gtcagatacc acataaacat atttatcttc caataccaat |
| 3241 | gtttgtttta ttaaaacaat aattaattaa taggctcaat agtgaccctc aagttaatcc |
| 3301 | catgacttcc ccaggctctg catgcataaa gaaatcattg tgacagaaaa atatcaacca |
| 3361 | catactgaag catagcttgc tagaacaaat cctgtacata tgaatgccag caactgggga |
| 3421 | cacatttagt attcacattt tatgcaaact tacttggtca attatttctt ttttaagcac |
| 3481 | aggtttattt tggaattcag ataagaactg acacatcata gacagtcatt gttaaggtaa |
| 3541 | ccctgttatt tcaattgcaa acaaacaata aacgtttttt cttcaa |

### EP300 is Required for High-Risk NB Growth

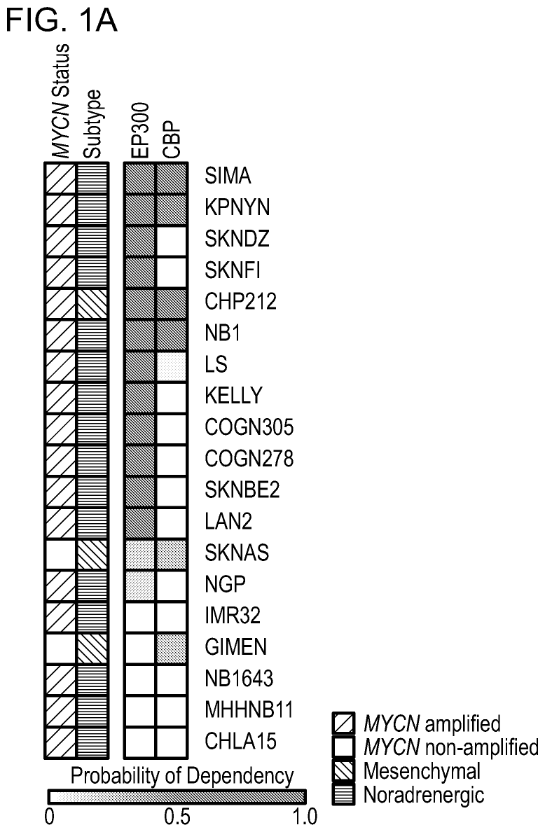

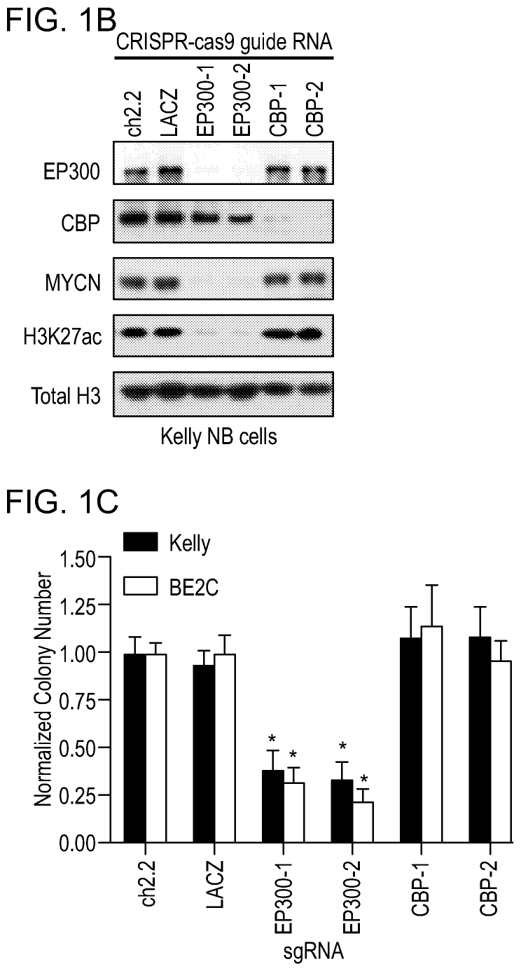

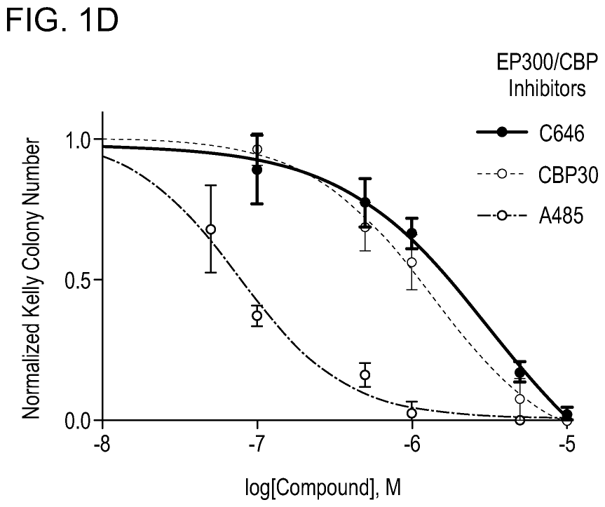

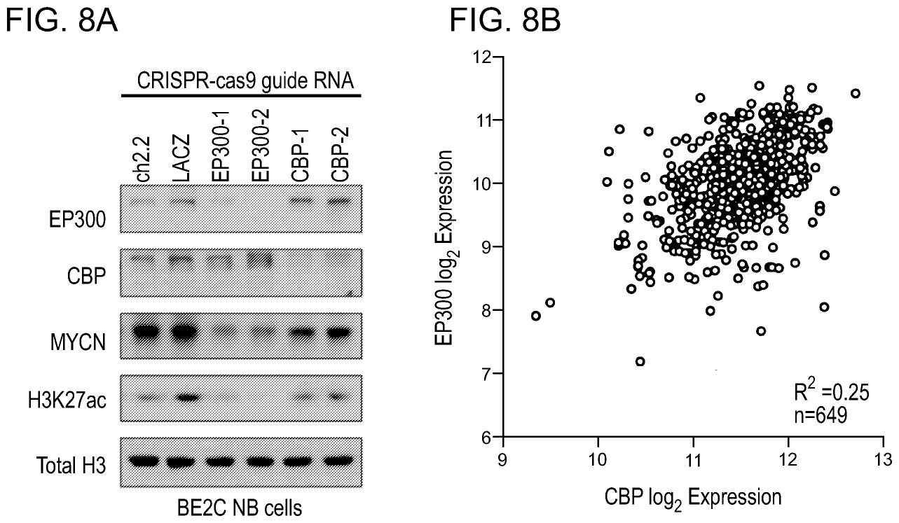

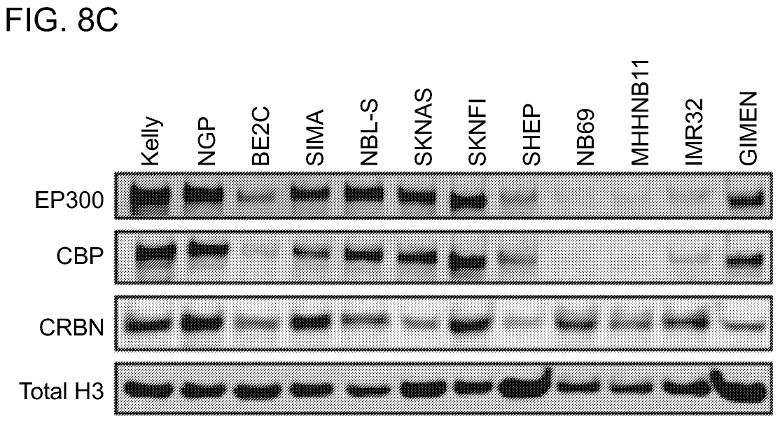

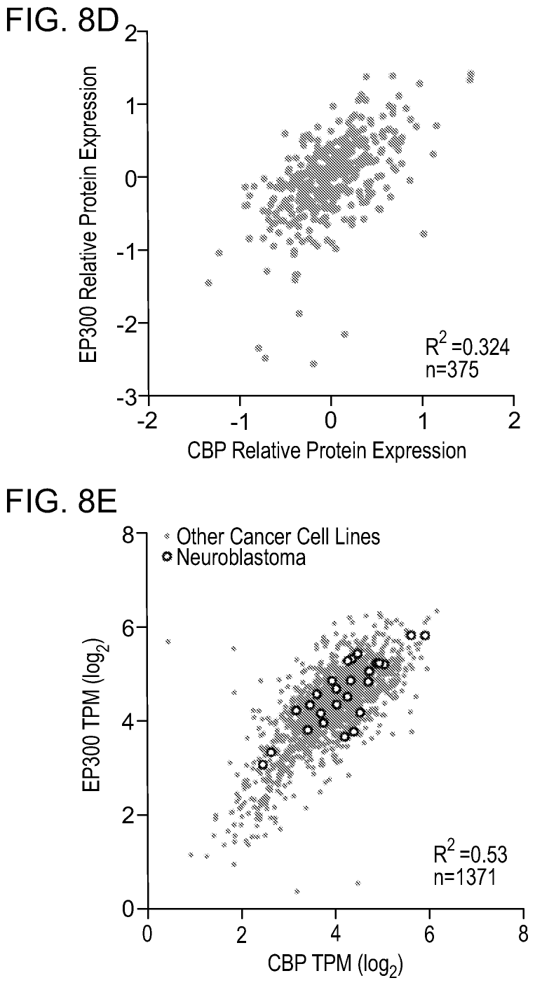

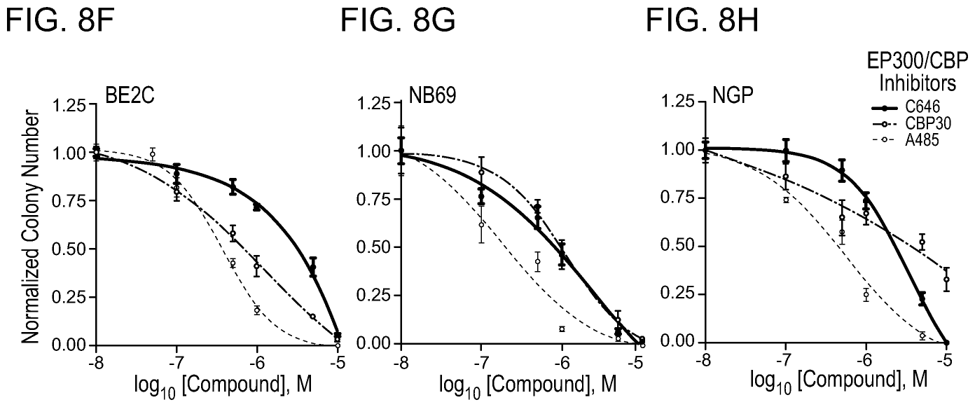

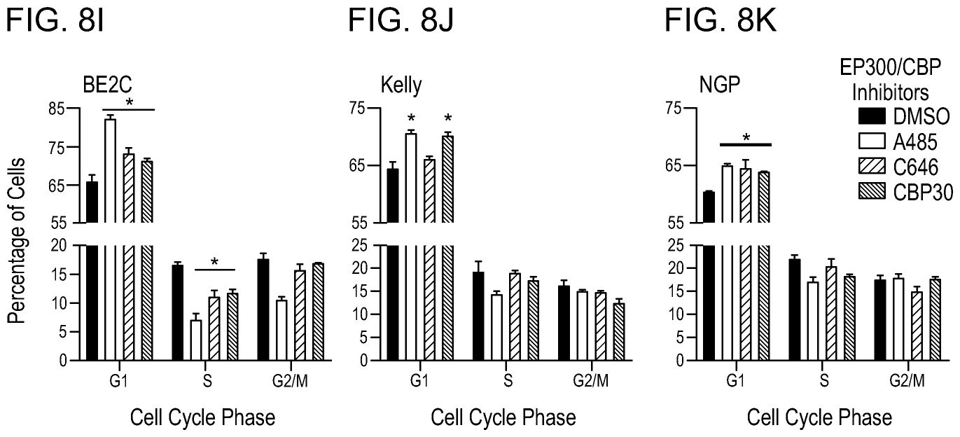

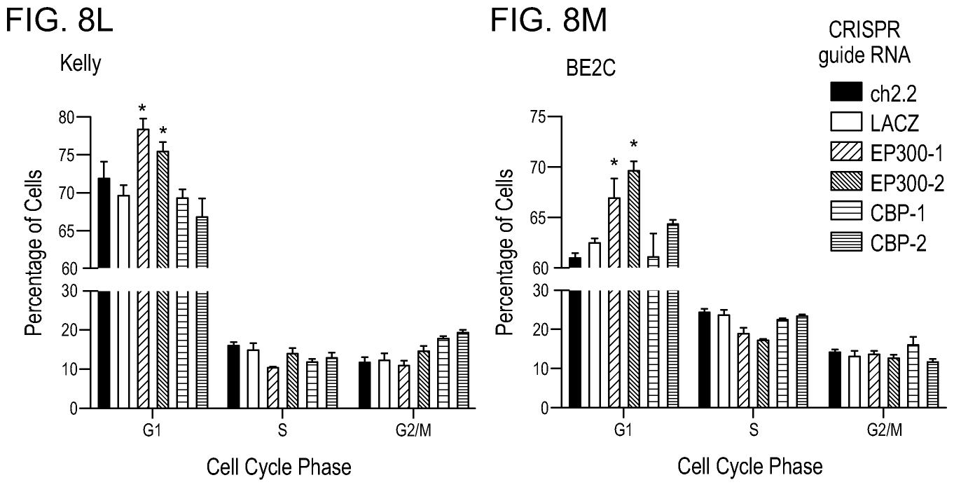

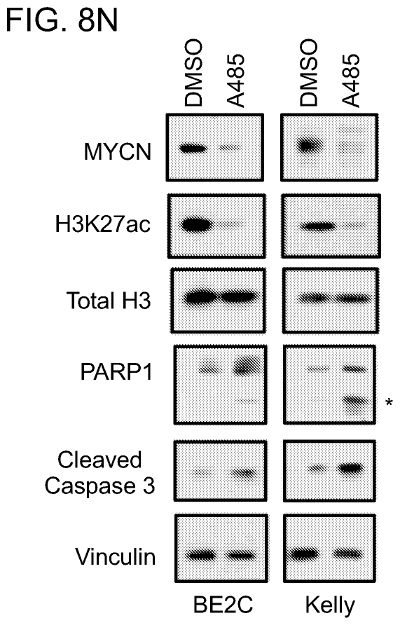

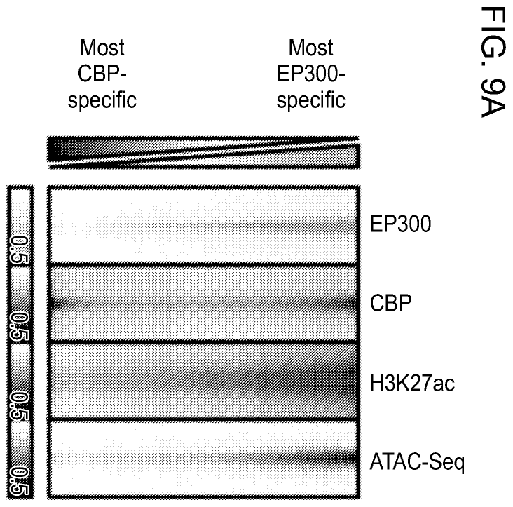

High-risk neuroblastoma requires a group of 147 genes for survival (Durbin et al. Nat. Genet. 50:1240-6 (2018)). One of these genes is the histone acetyltransferase enzyme EP300, but not its paralog CBP, which is surprising because EP300 is often redundant with CBP (Dancy and Cole, Chem. Rev. 115:2419-52 (2015)). Both EP300 and CBP acetylate the Lys-27 residue of histone H3 (H3K27ac), which is a mark associated with active gene transcription (Dancy and Cole, Chem. Rev. 115:2419-52 (2015); Durbin et al. Nat. Genet. 50:1240-46 (2018)). EP300, intriguingly, appeared to be uniquely required in neuroblastoma compared to CBP. Therefore, the relative expression and dependency of these two genes across a panel of representative neuroblastoma cell lines were investigated. First, the relative dependency of EP300 or CBP was examined in 19 high-risk neuroblastoma cell lines using the DepMap exome-wide Clustered Regularly Interspaced Short Palindromic Repeats (CRISPR)-CRISPR-associated protein 9 (Cas9) deletion dataset (Meyers et al. Nat. Genet. 49:1779-84 (2017)). Examination of the probability of dependency on EP300 and CBP in this panel of neuroblastoma cell lines demonstrated that the majority of cell lines require EP300 for cell growth (FIG. 1A). Interestingly, in four of the cell lines with a high level of dependency on EP300, dependency on CBP was also observed, indicating that each protein was essential to promote the expression of different subsets of genes (FIG. 1A). An additional four of the cell lines were not dependent on either EP300 or CBP, potentially indicating redundancy of these two acetyltransferases in these cell lines (FIG. 1A). To extend these findings, CRISPR-Cas9-mediated knockout of EP300 and CBP was performed in two MYCN-amplified NB cell lines, Kelly and BE2C (FIG. 1B, FIG. 8A). Although both EP300 and CBP are highly expressed in these two cell lines, loss of EP300 caused a profound loss of H3K27ac expression, while loss of CBP had a minimal effect, similar to control single-guide RNAs (sgRNAs), indicating that most enhancers and promoters rely on EP300 in these cell lines to catalyze H3K27ac. Further, expression of MYCN, which is a well-known dependency in MYCN-amplified neuroblastoma, was almost completely dependent on EP300 and not on CBP (FIG. 1B, FIG. 8A). Accordingly, CRISPR-Cas9 inactivation of EP300 markedly reduced colony formation in each cell line, while CBP inactivation did not (FIG. 1C). The residual colonies formed by EP300 CRISPR knockout cells did not express green fluorescent protein (GFP) which was co-expressed in the vector containing the guide RNA, indicating that they represent cells that were not infected with the vector containing EP300-targeted guide RNAs.

Analysis of EP300 and CBP messenger RNA (mRNA) expression in primary neuroblastoma tumors revealed a positive correlation in expression levels (FIG. 8B). Further, analysis of publicly available sequencing data in primary neuroblastoma tumors demonstrated that mutations in EP300 or CBP were rare in human high-risk neuroblastoma, including inactivating mutations (e.g., nonsense or frame-shift mutations) (Zhou et al. Nat. Genet. 48:4-6 (2016)). By western blotting, EP300 and CBP levels were generally similar to each other across a panel of neuroblastoma cell lines (FIG. 8C). Analysis of cancer cell lines in the Cancer Cell Line Encyclopedia (CCLE) proteomics and mRNA expression datasets also showed correlated expression levels of EP300 and CBP at both the RNA and protein levels, including the cell lines from patients with neuroblastoma (in red), indicating that these findings pertain across multiple tumor lineages (FIG. 8D-FIG. 8E).

To test the genetic findings using small molecule probes, we next performed colony formation assays of NB cells were performed with known combined inhibitors of EP300 and CBP , including two inhibitors targeting the HAT domain—A485 and C646, and one targeting the bromodomain—CBP30(Lasko et al. Nature 550:128-32 (2017); Yan et al. J. Invest. Dermatol. 133:2444-52 (2013); Hammitzsch et al. Proc. Natl. Acad. Sci. U.S.A. 112:10768-73 (2015)). These inhibitors are known to be nonselective between the two HATs. Across multiple NB cell lines, the most potent compound in reducing neuroblastoma colony formation was the HAT domain inhibitor A485 (FIG. 1D, FIG. 8F-FIG. 8H). Combined inhibition of EP300/CBP with A485 caused G1 cell cycle arrest within 24 hours (FIG. 8I-FIG. 8K), similar to the effects of knockout of EP300, but not CBP (FIG. 8LFIG. 8M). Prolonged treatment for seven days led to global loss of the H3K27Ac modification, loss of MYCN expression, and induction of cleaved caspase 3 and poly [ADP-ribose] polymerase 1 (PARP1), indicative of apoptotic cell death (FIG. 8N). Thus, both cell cycle progression and cell survival are impaired by inhibition of the HAT activity of both EP300 and CBP. The genetic studies indicated that this is due to a dependency on EP300, but not CBP, for the growth and survival of most MYCN-amplified neuroblastoma cell lines.

### EP300 Facilitates NB Adrenergic CRC-Driven Transcription Through Binding to TFAP2β

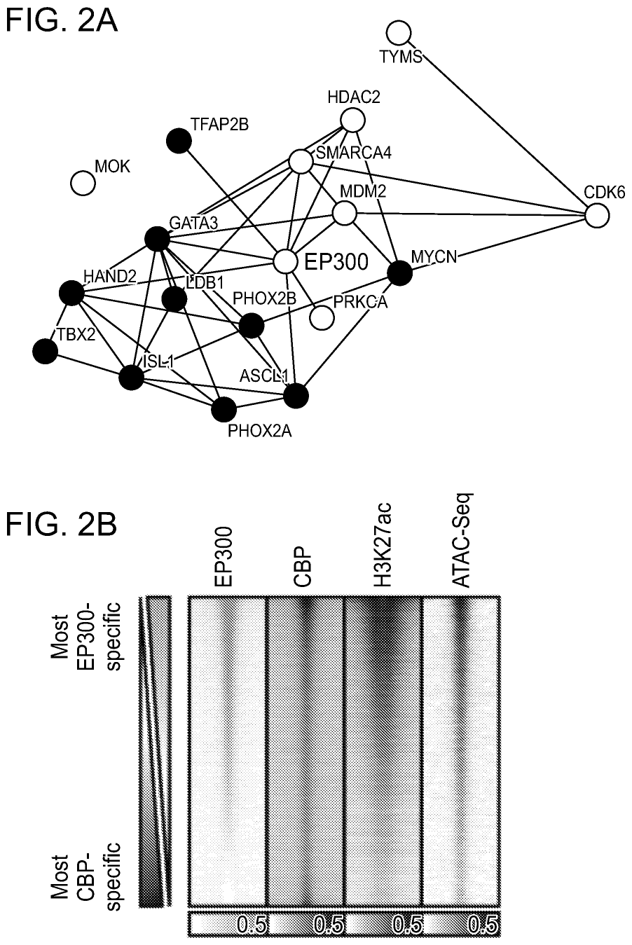

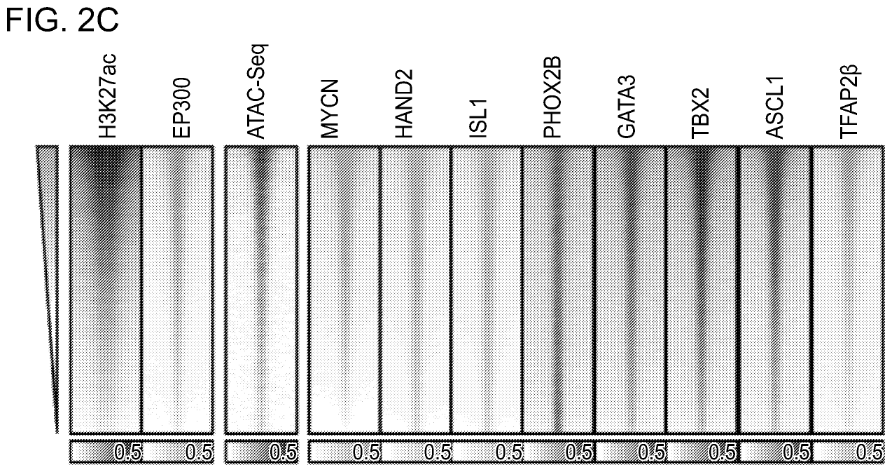

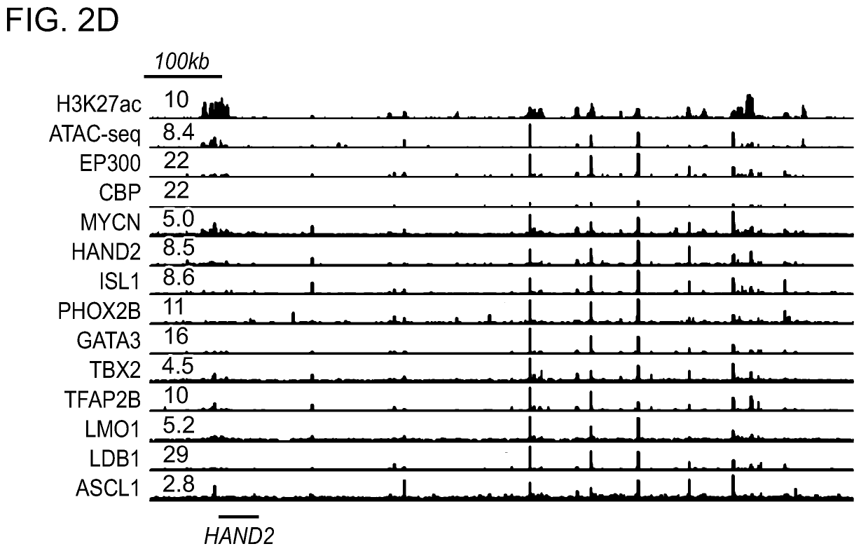

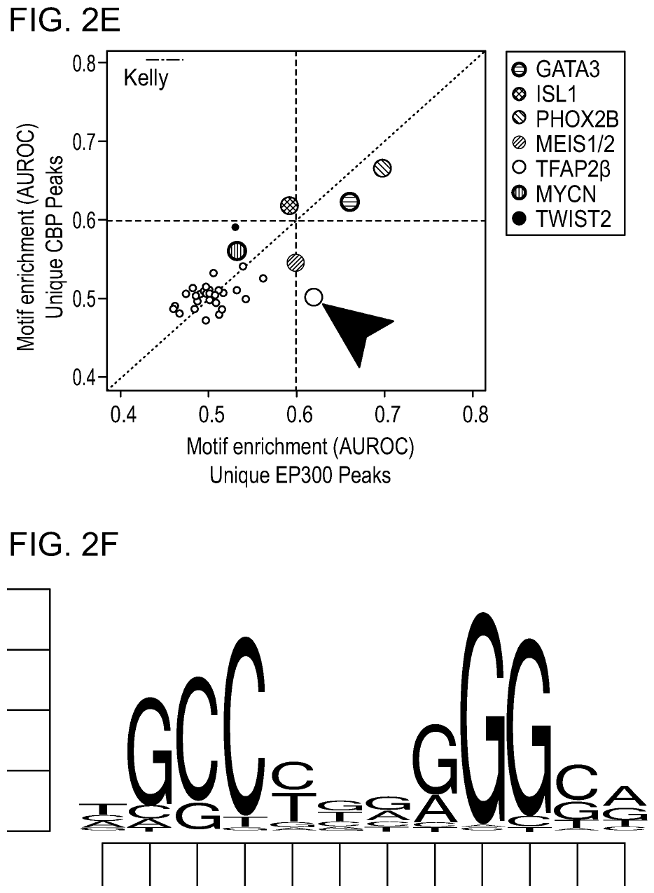

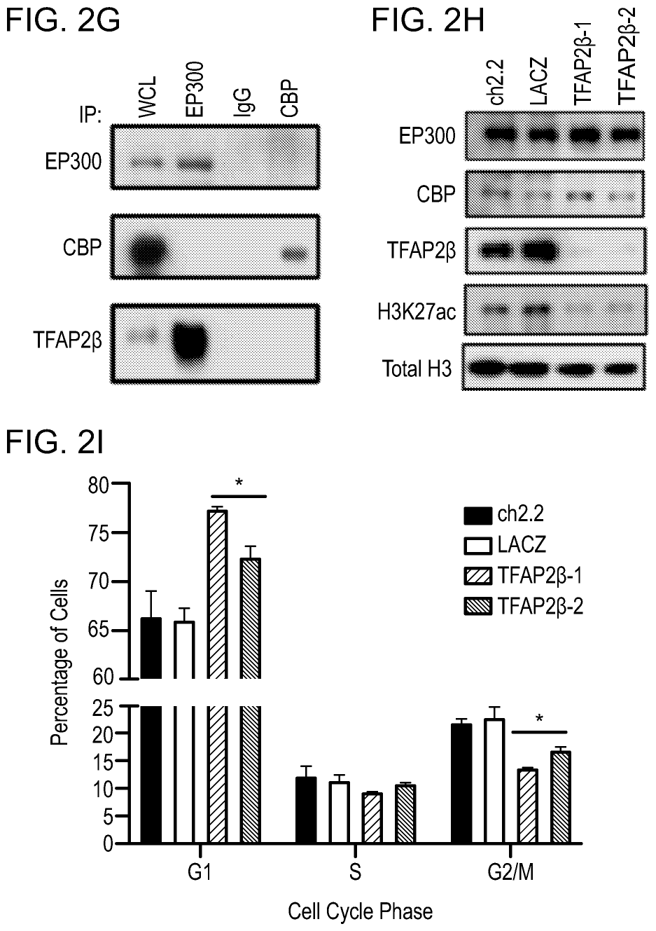

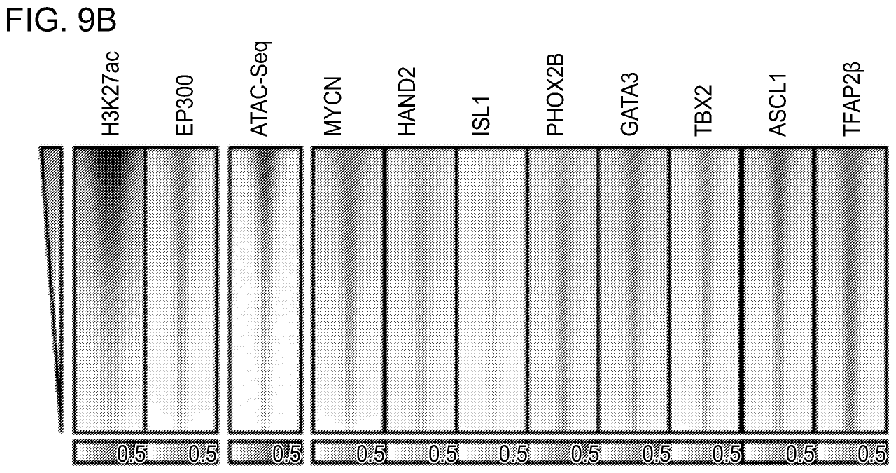

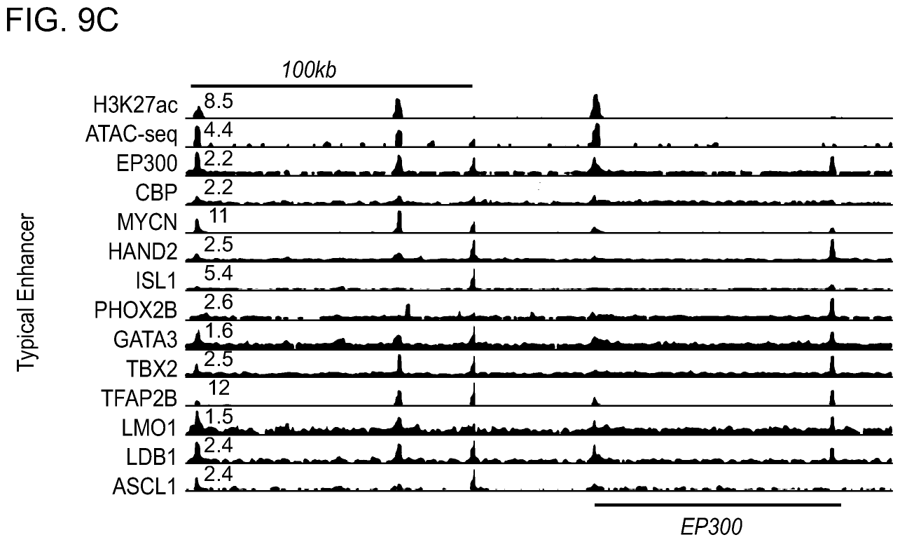

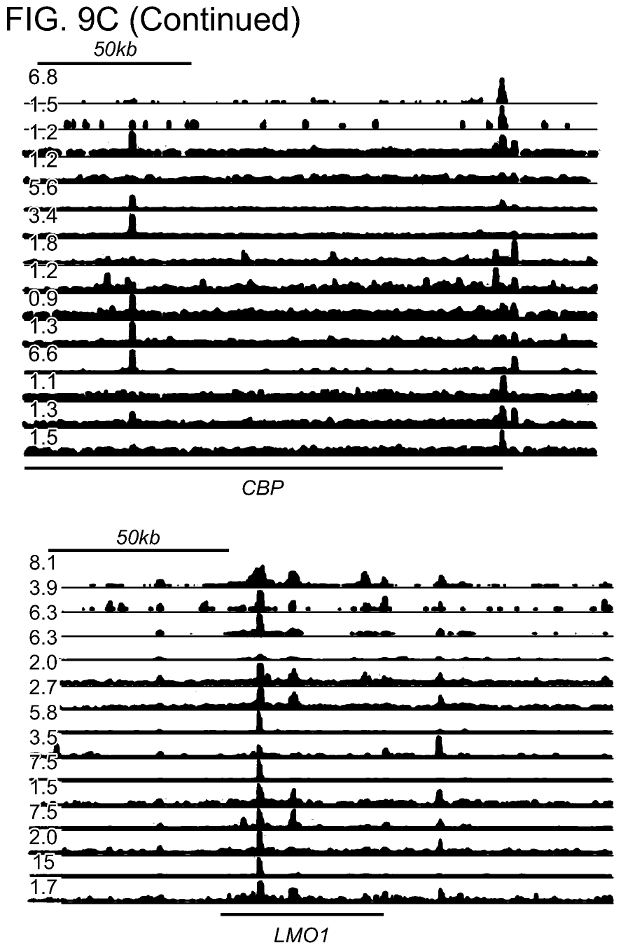

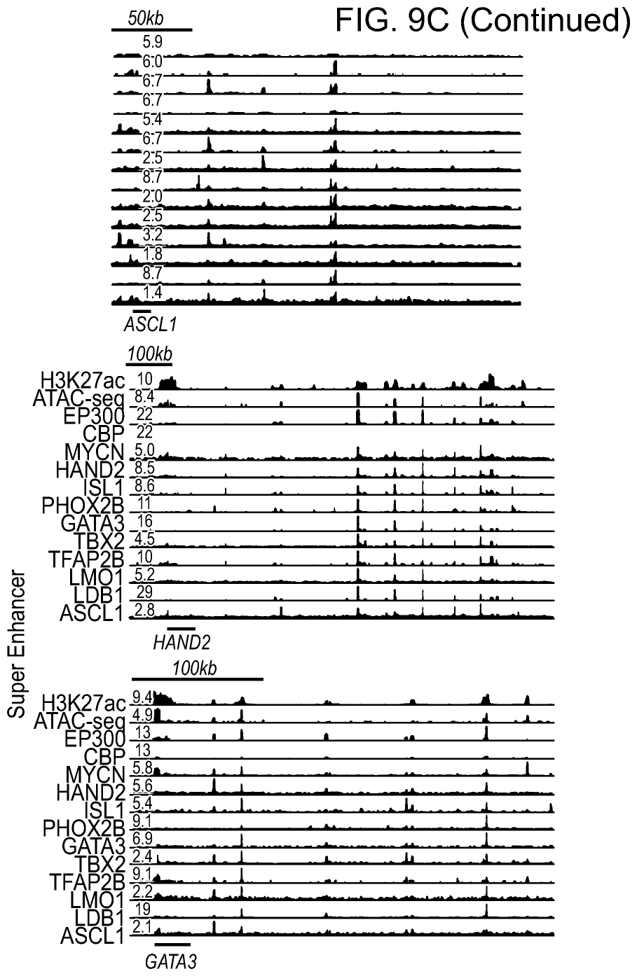

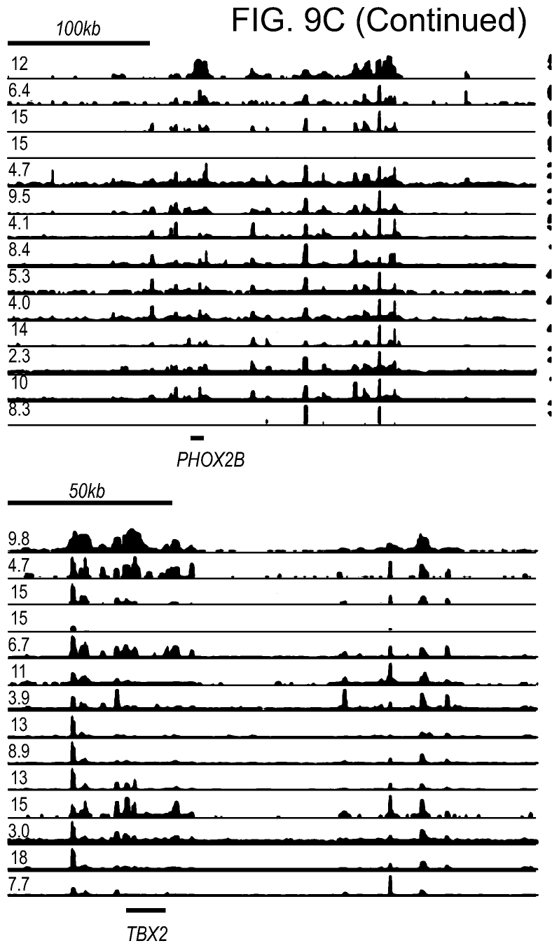

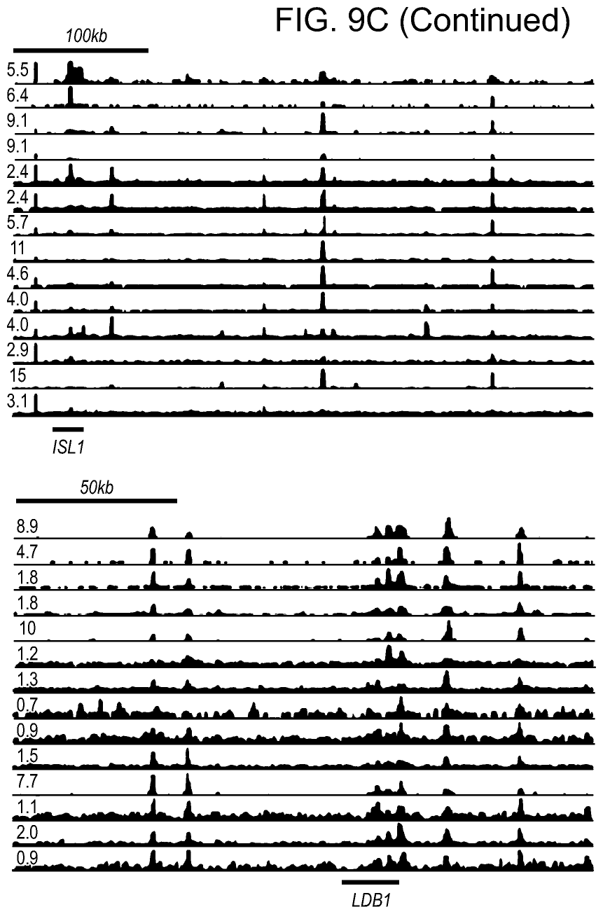

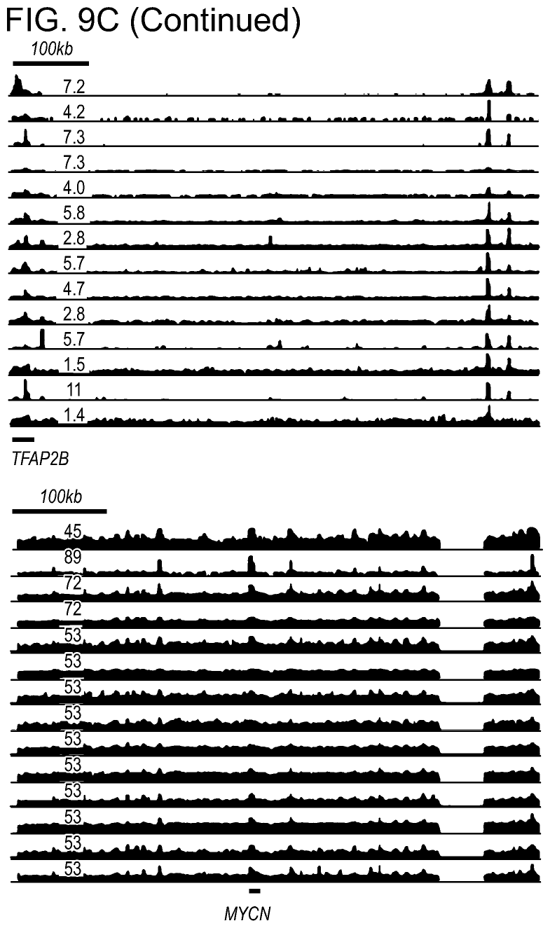

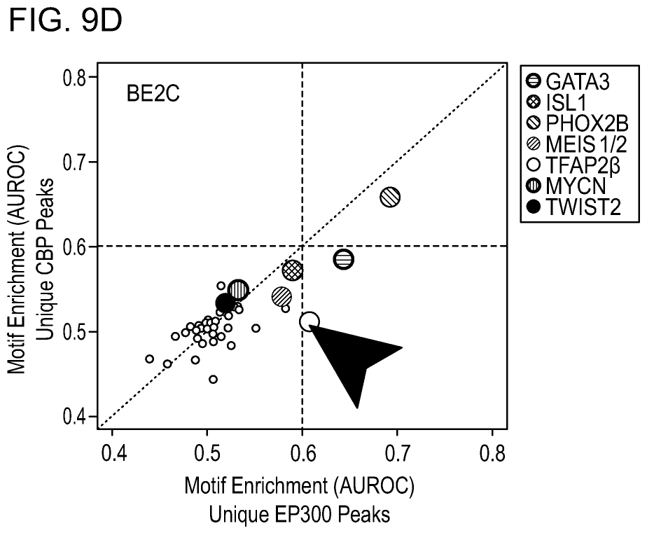

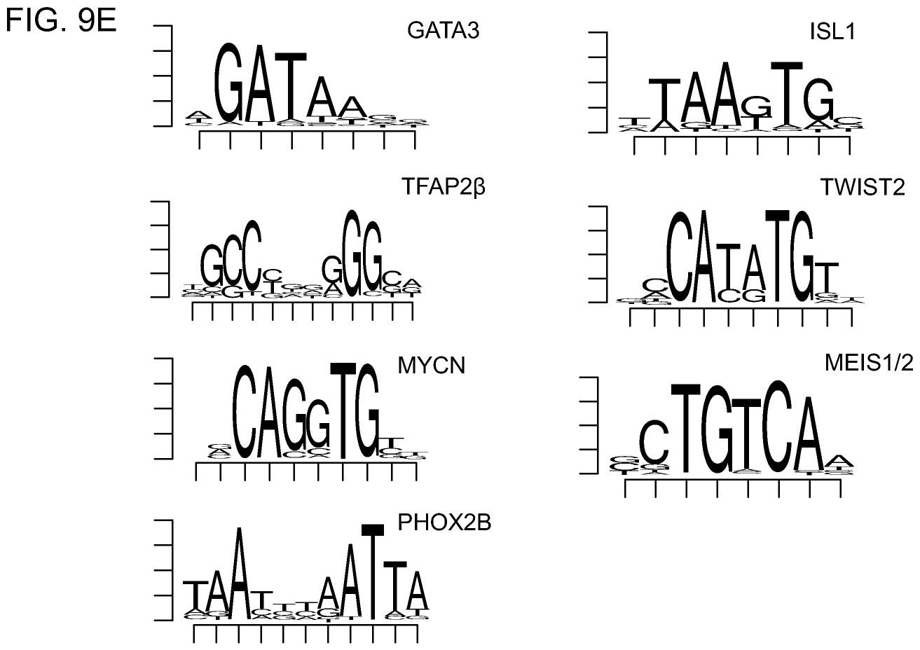

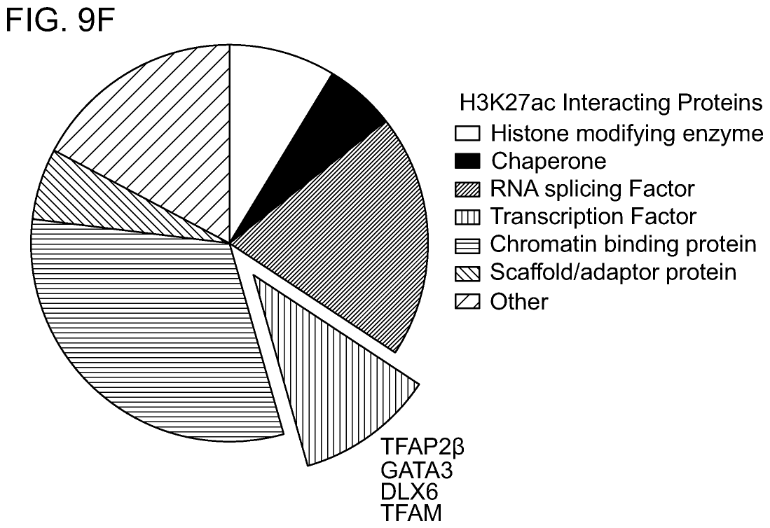

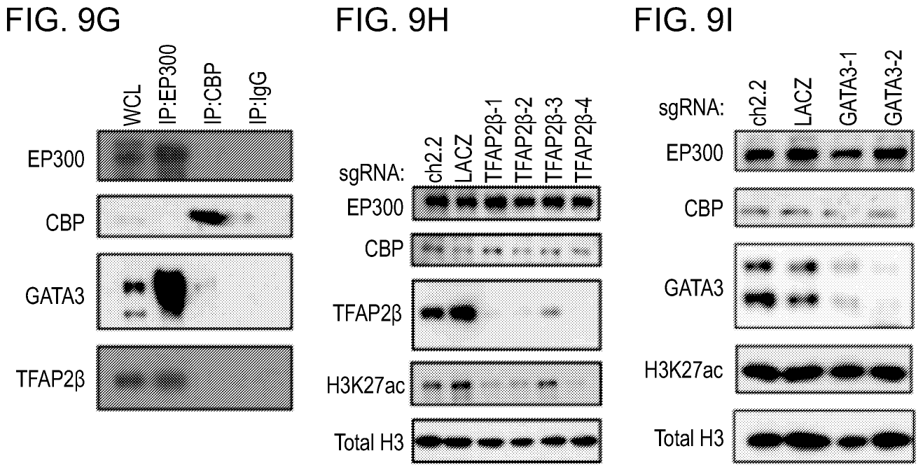

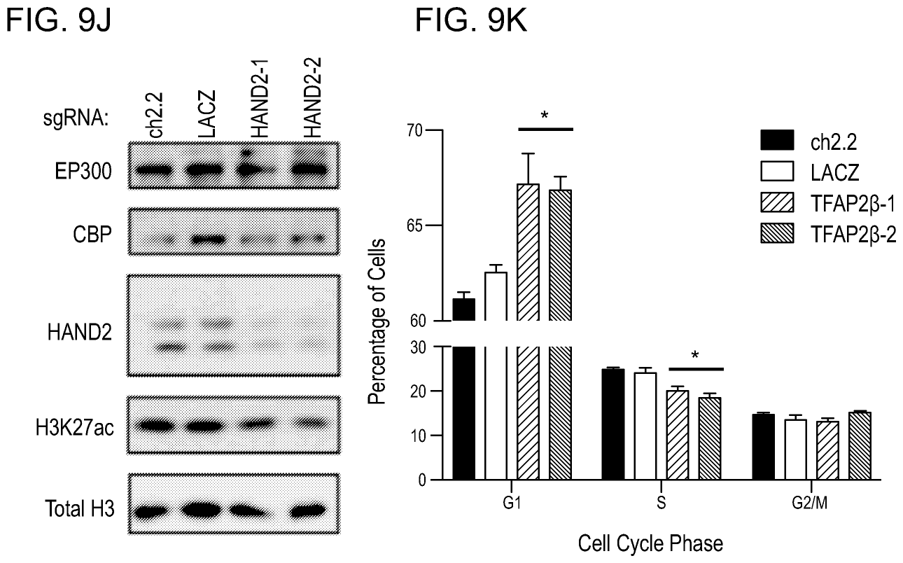

Next, the mechanism by which EP300, but not CBP, was required for growth of MYCN-amplified neuroblastoma cell lines was investigated. Core-regulatory circuitry (CRC) transcription factors (TF) were identified to be critically important in determining cell fate in neuroblastoma, and to be marked and regulated by extensive stretches of histone H3K27ac (Boeva et al. Nat. Genet. 49:1408-13 (2017); Durbin et al. Nat. Genet. 50:1240-46 (2018); van Groningen et al. Nat. Genet. 49:1261-6 (2017)). This analysis uncovered that the master transcription factors of adrenergic subtype NB include HAND2, ISL1, PHOX2B, GATA3, TBX2, and ASCL1. Thus, the mechanism by which EP300 collaborates with the NB CRC-driven gene expression program was also investigated. The Search Tool for the Retrieval of Interacting Genes/Proteins (STRING) database was used to perform an interaction analysis of all expressed nuclear neuroblastoma dependency genes (Szklarczyk et al. Nucleic Acids Res. 43:D447-52 (2015)). This analysis demonstrated that EP300 is found in a densely interacting network of genes, enriched for CRC transcription factors (FIG. 2A) (Durbin et al. Nat. Genet. 50:1240-6 (2018); Wang et al. Nat. Commun. 10:5622 (2019)). To understand the genome-wide binding patterns of EP300 and CBP, chromatin immunoprecipitation coupled to massively parallel high-throughput sequencing (ChIP-seq) experiments were performed using antibodies recognizing H3K27ac, EP300 and CBP in two separate MYCN-amplified neuroblastoma cell lines, BE2C and Kelly (FIG. 2B, FIG. 9A). This was also compared with Assay of Transposase Accessible Chromatin sequencing (ATAC-seq) data from both cell lines. Genome-wide heatmap analysis of binding demonstrated that EP300 displayed a similar pattern of binding as H3K27ac (FIG. 2B, FIG. 2A). In contrast, CBP binding demonstrated a distinct pattern, with enrichment at the regions most densely occupied by EP300, and many regions depleted of EP300 (FIG. 2B, FIG. 2A). The results show that EP300 primarily binds active enhancers that are marked by H3K27ac and accessible to transposase by ATAC-sequencing (FIG. 2B, FIG. 2A). Next, to examine the importance of EP300 in regulating gene expression programs in neuroblastoma cells, the degree of co-localization of EP300 was assayed with the targets of the previously defined neuroblastoma CRC transcription factors (FIG. 2C, FIG. 9B). Genome-wide heatmap analysis, ranked by binding of MYCN, demonstrated that EP300 displayed a similar pattern of binding as each of the CRC TFs (FIG. 2C, FIG. 9B). Specific evaluation of EP300 and CBP binding at specific CRC TF-encoding loci marked by dense H3K27ac signal demonstrated co-localization of EP300 with CRC transcription factors at sites bound by all CRC members (FIG. 2D, FIG. 9C, red tracks). CBP, in contrast, was minimally enriched at the loci of CRC transcription factors (FIG. 2D, FIG. 9C, green tracks). These data indicate that EP300, but not CBP, is preferentially localized across the genome at open chromatin sites densely enriched for binding of CRC factors, including sites within the enhancers controlling adrenergic CRC genes themselves and the enhancers of genes comprising the extended regulatory network of the adrenergic CRC in NB cells (Durbin et al. Nat. Genet. 50:1240-6 (2018)).

Both EP300 and CBP lack sequence-specific DNA binding activity and require association with a DNA-binding factor to achieve locus-specific binding (Song et al. Biochem. Biophys. Res. Commun. 296:118-24 (2002)). Thus, the mechanism by which EP300 is targeted to chromatin loci associated with enhancers of the CRC was investigated. To identify proteins involved in EP300 recruitment to DNA in NB cells, a motif enrichment analysis of the top 500 peaks bound by either EP300 or CBP was performed in Kelly and BE2C NB cell lines. Consistent with prior evidence indicating that EP300 proteins form interactions with several TFs to nucleate higher order enhance some structures, this analysis demonstrated enrichment for several transcription factor consensus binding motifs preferentially associated with either EP300 or CBP binding (FIG. 2E-FIG. 2F, FIG. 9D-FIG. 9E) (He et al. Nucleic Acids Res. 39:4464-74 (2011)). Two motifs were selectively enriched under EP300 bound peaks in both cell lines, corresponding to consensus binding sequences for the transcription factors GATA3 and TFAP2β (FIG. 2E-FIG. 2F, FIG. 9D-FIG. 9E). To validate that these transcription factors associate with H3K27ac-marked chromatin, co-immunoprecipitation of H3K27ac of nuclear extracts of Kelly and BE2C cells was next performed, followed by mass spectrometry of the isolated protein. As expected, peptides corresponding to EP300 and CBP that co-immunoprecipitated with H3K27ac were detected in both Kelly and BE2C cells. However, this experiment also identified that four transcription factors, including GATA3 and TFAP2β, physically interact with H3K27ac-marked nucleosomes in both cell lines (Table 3, Example 5, and FIG. 9F). GATA3 is a known member of the CRC of high-risk NB cells (Boeva et al. Nat. Genet. 49:1408-13 (2017); Durbin et al. Nat. Genet. 50:1240-6 (2018)). TFAP2β has been identified as a possible CRC member since it is a transcription factor dependency in NB commonly regulated by a super-enhancer (Boeva et al. Nat. Genet. 49:1408-13 (2017); van Groningen et al. Nat. Genet. 49:1261-6 (2017); Durbin et al. Nat. Genet. 50:1240-6 (2018)). Using new antibodies against TFAP2β, ChIP-seq for TFAP2β binding was performed, and the results show that TFAP2β binds to the super-enhancers and regulates other members of the CRC (FIG. 2B-FIG. 2C, FIG. 9B-FIG. 9C). Thus, TFAP2β represents a newly identified member of the adrenergic core-regulatory circuitry in MYCN-amplified NB cells.

Because EP300 binding was enriched at sites containing GATA3 and TFAP2β motifs, and all these proteins bound to H3K27ac-marked chromatin, the physical association of EP300 with GATA3 and TFAP2β was then investigated. Immunoprecipitation of EP300 and CBP in Kelly NB cells, followed by western blotting for TFAPP and GATA3 demonstrated that EP300, but not CBP, physically interacts with both TFAPP and, to a lesser degree, GATA3 (FIG. 2G, FIG. 9G). Additionally, reciprocal co-immunoprecipitation of TFAP2β in Kelly cells demonstrated the presence of EP300 proteins, but not CBP (FIG. 9G). To determine whether these transcription factors are able to control localization of EP300, CRISPR-Cas9-based knockout of TFAP2β or GATA3 was performed in Kelly NB cells (FIG. 2H, FIG. 9H-FIG. 9I). As a control, knockout studies of HAND2, a CRC factor that did not display selective motif enrichment under EP300 peaks, were also performed (FIG. 9J). Day 5 after knockout of TFAP2β, loss of TFAP2β and H3K27ac expression levels was observed without effects on expression levels of EP300 or CBP (FIG. 2H, FIG. 9H). In contrast, knockout of either GATA3 or HAND2 had no effect on the levels of H3K27ac (FIG. 9I-FIG. 9J). As with knockout of EP300, knockout of TFAP2β also resulted in G1 cell cycle arrest in Kelly and NGP NB cells (FIG. 2I, FIG. 9K). Thus, these data indicate that EP300 is targeted to DNA through a physical interaction with the CRC transcription factor TFAP2β in neuroblastoma.

### EP300 was Selectively Degraded by Proteolysis Targeting Chimera (PROTAC®), JQAD1

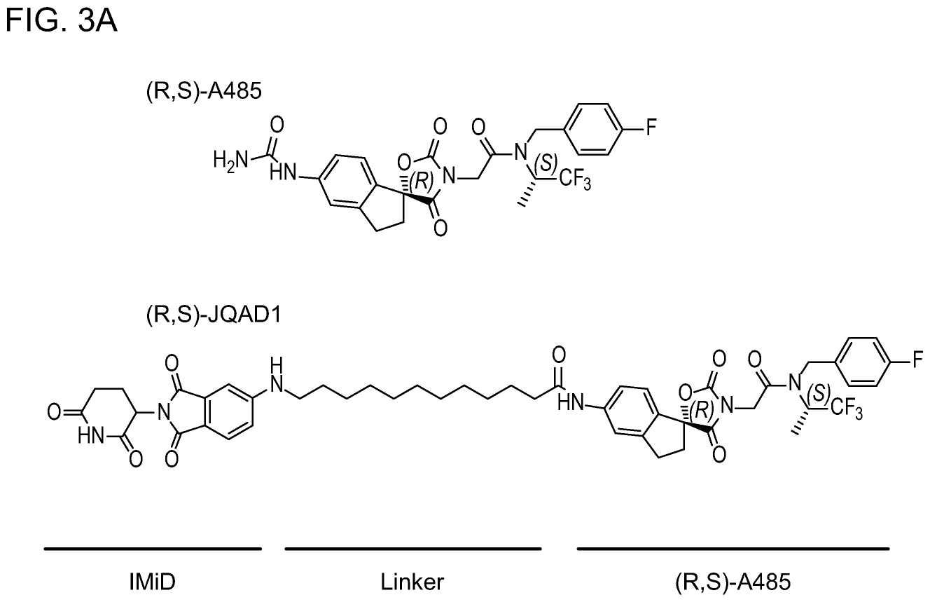

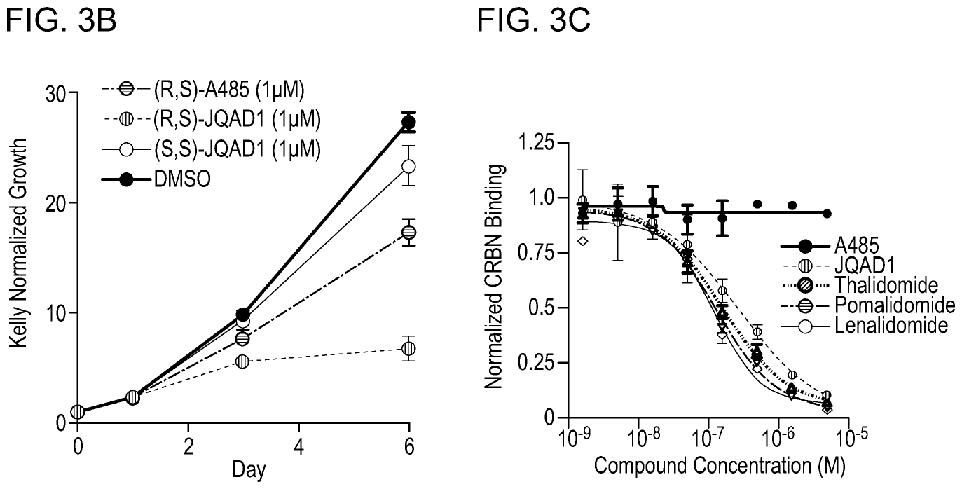

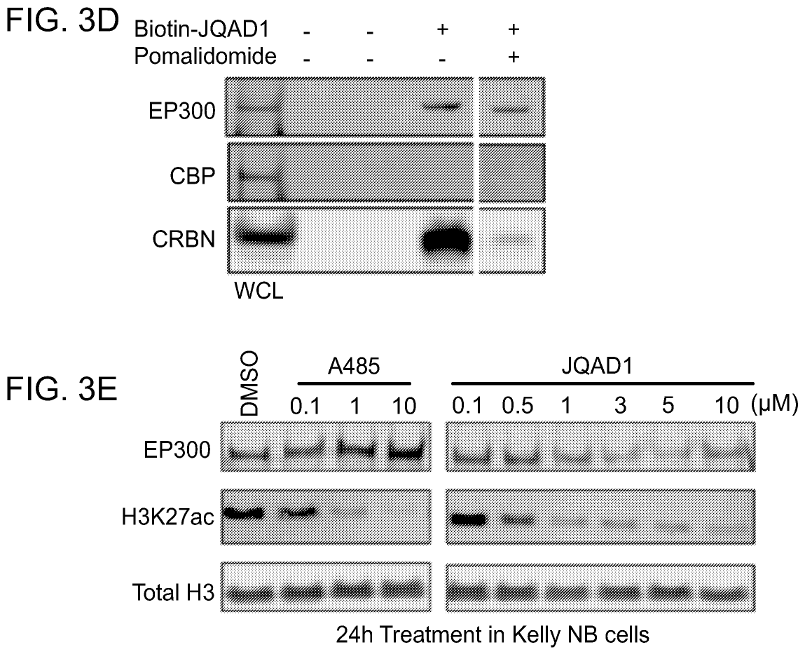

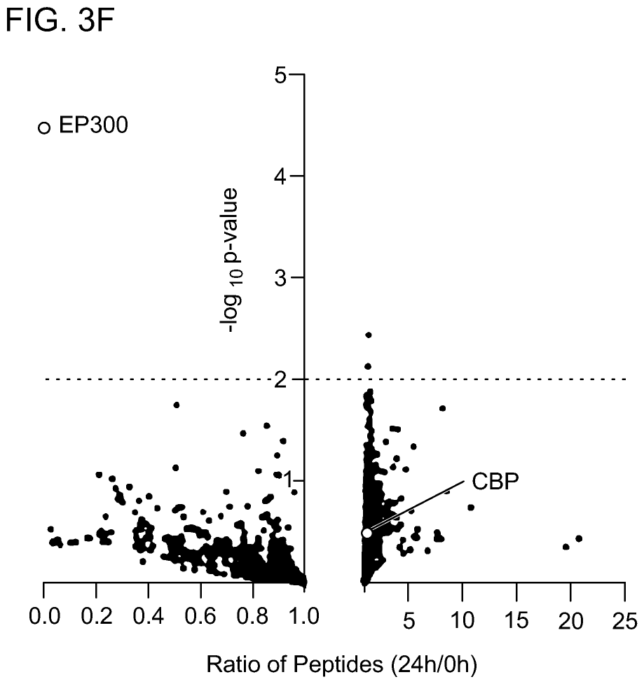

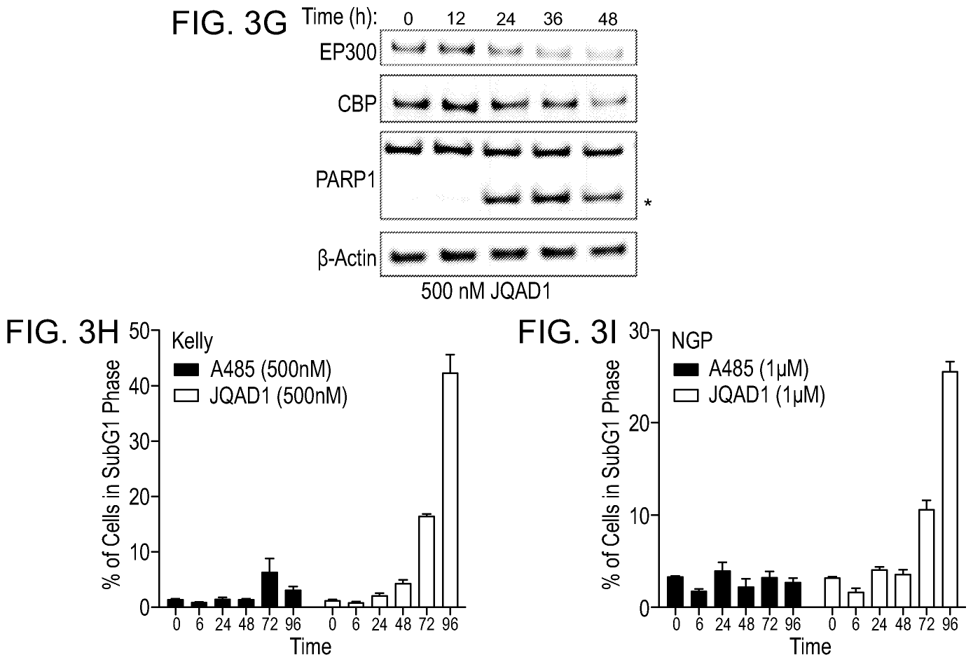

All small molecules that are currently available and inhibit the HAT activity of EP300 also inhibit the HAT activity of CBP with nearly an equivalent K_{d} (Dancy and Cole, Chem. Rev. 115:2419-52 (2015); Hammitzsch et al. Proc. Natl. Acad. Sci. U.S.A. 112:10768-73 (2015); Lasko et al. Nature 550:128-32 (2017); Yan et al. J. Invest. Dermatol. 133:2444-52 (2013)). This includes A485, the most potent and specific HAT inhibitory compound developed to date (Lasko et al. Nature 550:128-32 (2017); Michaelides et al. ACS Med. Chem. Lett. 9:28-33 (2018)). One approach to selectively target EP300 in neuroblastoma may be to disrupt the interaction between TFAP2β and EP300, however, a strategy like this has typically been difficult to implement (reviewed in Wimalasena et al. Mol. Cell. 78:1086-95 (2020)). Recently, evidence has indicated that an alternative approach to develop selective compounds may be through the development of small molecule degraders, termed “PROTAC® s.” PROTAC® s are heterobifunctional small molecules that bind the target protein and mediate the formation of a ternary complex between the target protein and an E3 ligase receptor (reviewed in Burslem and Crews, Cell 181:102-14 (2020)). The ternary complex formed by the PROTAC® and the target protein bridges to an E2 ubiquitin ligase, which polyubiquitinates the target protein and directs it to the proteasome for degradation and recycling (Burslem and Crews, Cell 181:102-14 (2020)). To this end, the degrader molecule A485 has been reported that degrades EP300 and CBP indiscriminately, using a bait molecule that targets the bromodomain of these proteins (Vannam et al. Cell Chem Biol, 28:503-14.e12 (2020)). However, since A485 was the most potent small molecule inhibitor in neuroblastoma cells and has the lowest K_{d} value for EP300 and CBP of all small molecules targeting these proteins, the activity of a small molecule degrader using A485 as a bait molecule was therefore tested (FIG. 3A) (Lasko et al. Nature 550:128-32 (2017)). Computational structural modeling of the interaction between the HAT domain of EP300 and the immunomodulatory imide drug (iMiD) binding region of CRBN indicated that an optimal linker length between A485 and the E3 ligase would be between 8-12 carbons. Therefore, compound JQAD1, containing the two chiral centers found within the A485 molecule and a 12-carbon linking chain, was designed, and synthesized (FIG. 3A, Scheme 1) (Michaelides et al. ACS Med. Chem. Lett. 9:28-33 (2018)).

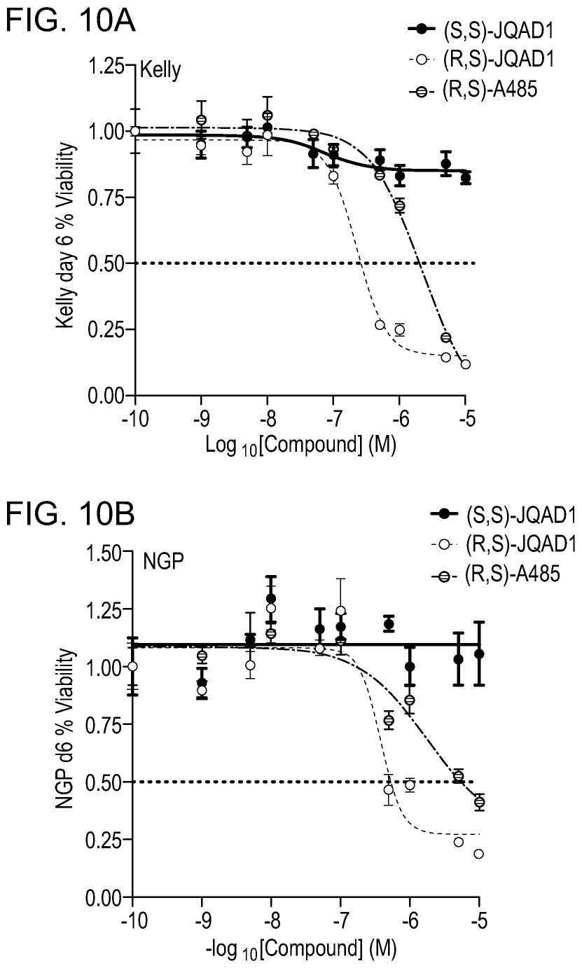

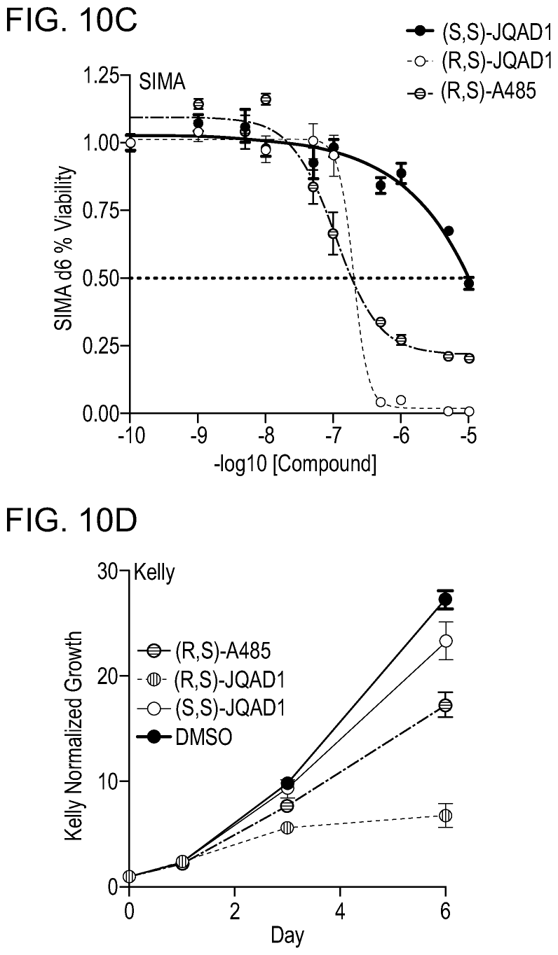

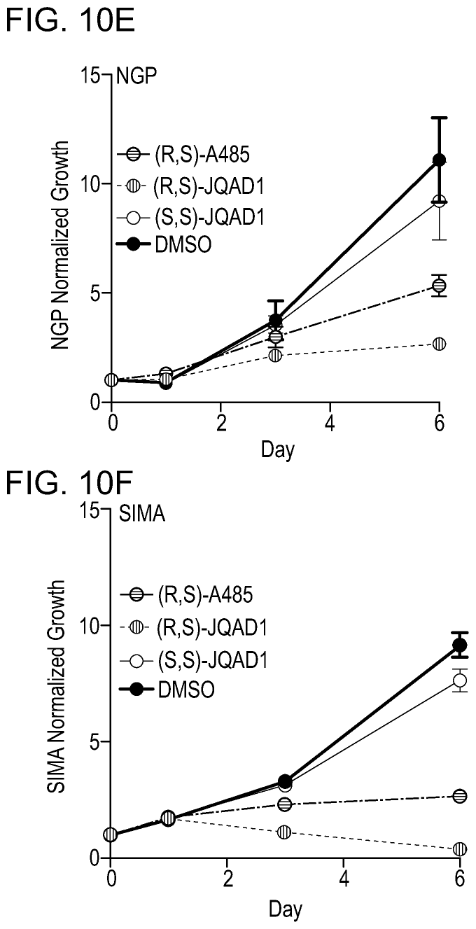

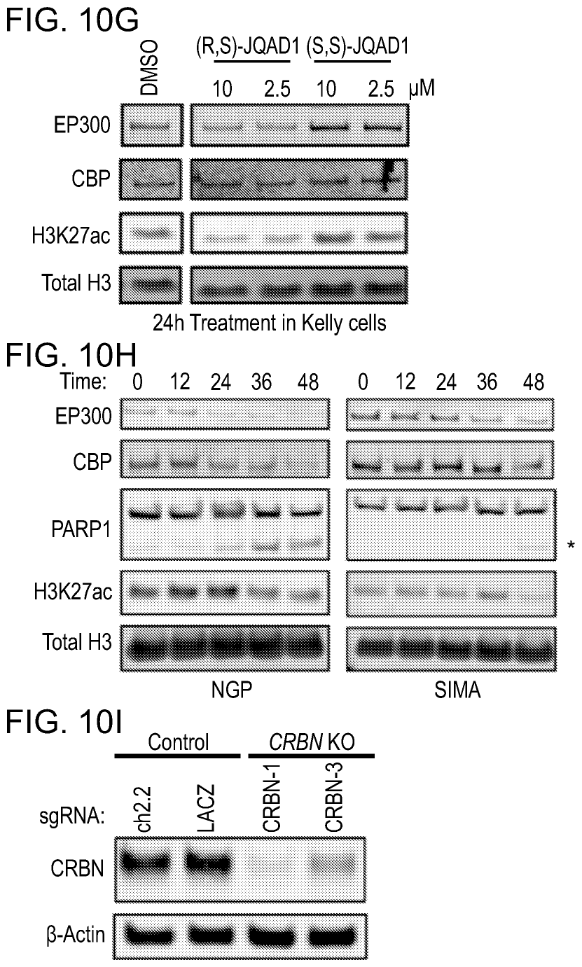

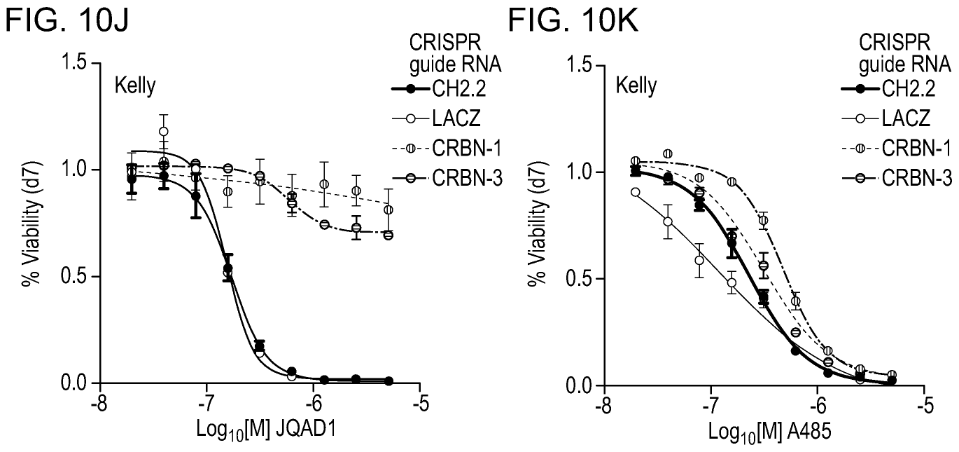

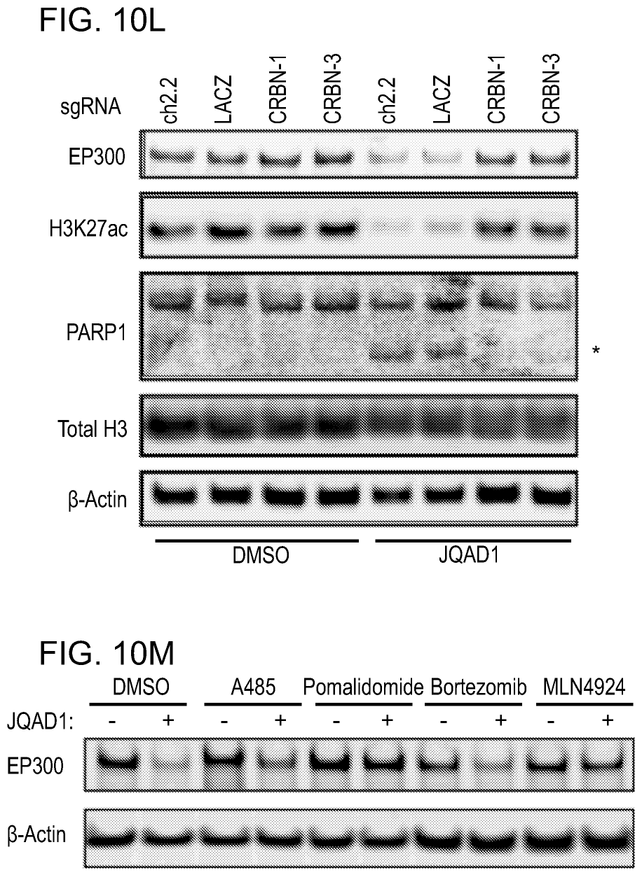

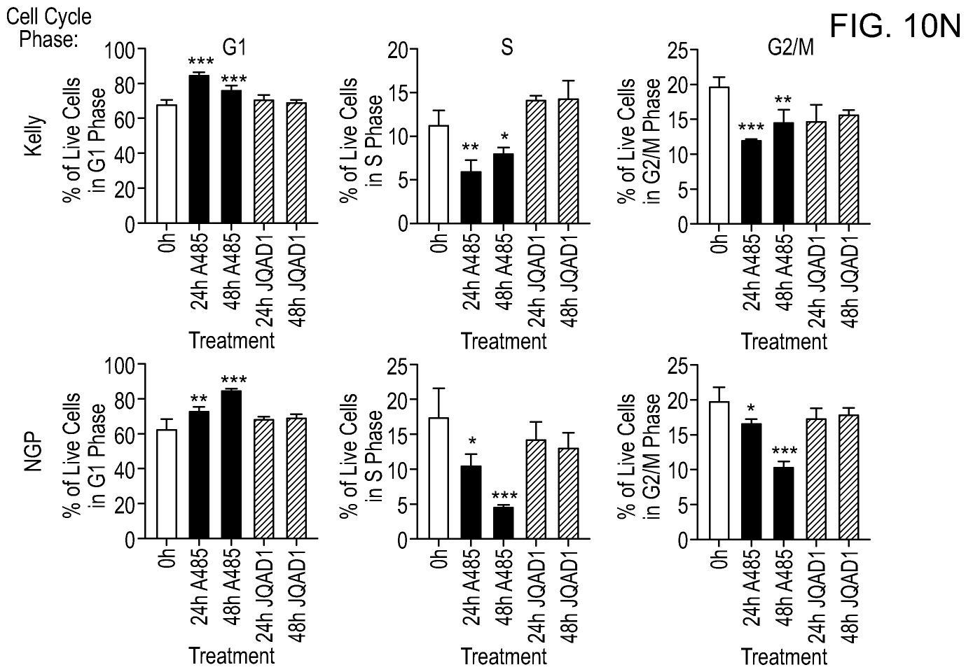

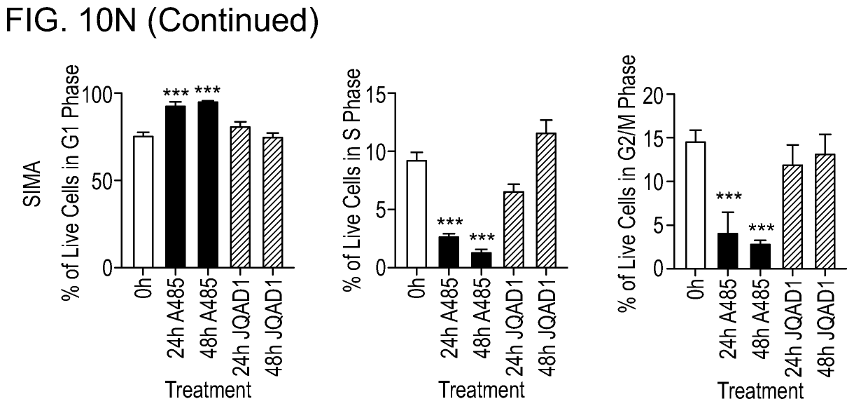

Kelly, NGP, and SIMA, three neuroblastoma cell lines that express high levels of CRBN, were treated with purified (R,S) and (S,S) stereoisomers of JQAD1 (FIG. 8C). Comparison established that the (R,S) stereoisomer had the lowest IC50 concentration in intact Kelly, NGP and SIMA NB cells, and that this IC₅₀ was lower than that of the parental molecule A485 (FIG. 3B, FIG. 10A-FIG. 10F). Therefore, the term JQAD1, as used herein, refers to the more active (R,S) stereoisomer of the PROTAC® compound, unless the (S,S) stereoisomer is used as a control and specifically indicated.

To determine whether JQAD1 interacts with the E3 ligase receptor CRBN, the AlphaLISA® platform was used to perform AlphaLISA® fluorescent assays using biotinylated pomalidomide bound to beads and His-tagged CRBN (Yasgar et al. Methods Mol. Biol. 1439:77-98 (2016)). All iMiD-containing compounds, including JQAD1 and free pomalidomide, efficiently interacted with CRBN in the AlphaLISA® assays, while the parental compound A485 did not (FIG. 3C). Next, biotinylated JQAD1 (Biotin-JQAD1, Scheme 2) was synthesized and incubated with Kelly cell lysates, followed by streptavidin-based purification. Western blotting of purified lysates demonstrated the presence of EP300 and CRBN, but surprisingly not CBP proteins (FIG. 3D). Furthermore, co-treating Kelly cell lysates with JQAD1 and excess pomalidomide (to compete for binding to CRBN) resulted in a partial loss of the interaction between JQAD1, CRBN, and EP300, indicating that these three proteins form a ternary complex (FIG. 3D). Surprisingly, Applicant discovered that PROTAC® JQAD1 specifically binds EP300, the dominant mediator of H3K27ac in high-risk neuroblastoma. PROTAC®s may acquire preferential specificity for one of two possible target enzymes due to restricted three-dimensional interactions, as noted in Burslem and Crews, Cell 181:102-14 (2020). In this case, the preferential targeting of EP300 by PROTAC® JQAD1 is clearly an advantage over prior compounds, as neuroblastoma cells are often exclusively dependent on EP300, while normal cells of different lineages may require CBP, and thus would be spared from toxicity by the specificity of PROTAC® JQAD1.

Because JQAD1 interacted preferentially with both EP300 and CRBN, whether JQAD1 preferentially induces degradation of EP300 compared to CBP in MYCN-amplified neuroblastoma cells was examined. Treatment of Kelly cells for 24 hours (h) with JQAD1 demonstrated a dose-dependent decrease in EP300 expression, along with a parallel loss of the H3K27ac modification (FIG. 3E). Similar treatment of Kelly cells with A485 caused a loss of H3K27ac, due to catalytic inhibition of EP300 enzymatic activity (FIG. 3E). Treatment of Kelly cells with (R,S)-JQAD1 and the control (S,S)-JQAD1 for 2 4h revealed that (S,S)-JQAD1 had limited effects on H3K27ac or EP300 expression levels, while (R,S)-JQAD1 suppressed both H3K27ac and EP300 expression levels (FIG. 10G). Consistent with the specificity of JQAD1 for EP300, neither compound had significant effects on expression levels of CBP at this time point (FIG. 10G). To further examine the specificity of JQAD1 for EP300, an analysis of the effects of JQAD1 in stable isotope labeling by amino acids in cell culture (SILAC)-labelled Kelly cells was performed (FIG. 3F). Kelly cells were cultured with SILAC media containing heavy or light-labelled arginine and lysine. Heavy labelled cells were treated with 500 nM JQAD1, and light-labelled cells treated with dimethyl sulfoxide (DMSO) for 24 h, prior to nuclear extraction and protein lysis. As a control, nuclear extraction and lysis on untreated heavy and light-labelled Kelly cells were performed. Protein abundance was then analyzed by mass spectrometry, to determine global changes in the nuclear proteome. Following 24 h of treatment with JQAD1, EP300 protein was significantly decreased (p=3.3×10⁻⁵), while CBP and other proteins within the nuclear proteome remained detectable at similar levels as controls (FIG. 3F). To expand upon these findings, three NB cell lines, Kelly, NGP and SIMA, which have high levels of CRBN (e.g., protein or mRNA) (FIG. 8C), were treated with JQAD, and the effects on specific proteins were measured by western blotting. In all three cell lines, JQAD1 induced selective loss of EP300 expression by 24 h, coincident with cleavage of PARP1, signaling the onset of apoptosis (FIG. 3G, FIG. 10H). At this time point in all three cell lines, CBP could still be detected. With extended treatment, loss of CBP expression was observed, though this could not be separated from general effects of apoptosis (FIG. 3G, FIG. 10H).

JQAD1 contains an IMiD moiety which interacts with the E3 ligase receptor CRBN (FIG. 3C-FIG. 3D). To demonstrate genetically that CRBN is required for JQAD1-mediated EP300 degradation and cellular effects, CRISPR-Cas9 gene editing was used to produce Kelly cells with stable disruption of the CRBN gene. Western blotting of lysates prepared from control or CRBN-edited Kelly cells demonstrated loss of CRBN expression in CRBN-edited cells with retained expression in control-edited cells (FIG. 10I). Control-edited Kelly cells were potently killed by JQAD1; however, CRBN-knockout cells were resistant to the effects of JQAD1, indicating that CRBN expression was required for JQAD1 growth suppressive activity (FIG. 10J). By contrast, A485 equivalently inhibited the growth of both CRBN-knockout cells and controls (FIG. 10K), indicating that loss of CRBN has no effect on the enzymatic function of EP300. Further, western blotting of lysates prepared from control and CRBN-knockout Kelly cells treated with JQAD1 or DMSO demonstrated that JQAD1 suppressed EP300 expression and the H3K27ac modification, and induced apoptosis marked by PARP1 cleavage in control knockout cells, but not in CRBN-edited cells (FIG. 10L). Thus, CRBN is required for JQAD1-mediated EP300 degradation and the induction of apoptosis. Since treatment of CRBN-edited cells with JQAD1 had no effect on H3K27ac, the structure of JQAD1 prevents its A485 moiety from competitively inhibiting EP300 HAT activity, and, therefore, acts as a pure CRBN-dependent protein degrader without catalytic inhibitory activity (FIG. 10L). To further probe the pathway involved in JQAD1-mediated EP300 degradation, western blotting was performed on Kelly cells co-treated with JQAD1 and other compounds predicted to disrupt JQAD1 function. Degradation of EP300 was blocked by co-treatment with excess A485 or IMiD (pomalidomide), or with inhibitors of the proteasome (bortezomib) or neddylation of E3 ubiquitin ligases (MLN4924) (FIG. 10M). These data indicate that JQAD1 functions by binding to EP300, which leads to its CRBN-dependent proteasomal degradation.

JQAD1 resulted in potent CRBN- and proteasomal-dependent loss of EP300 and cell death. To evaluate the mechanism by which JQAD1 reduced cell growth, Kelly and NGP cells were treated with JQAD1, A485, or vehicle control, and propidium-iodide DNA flow cytometry was performed. Cells treated with A485 displayed a phenotype of G1 cell cycle arrest (FIG. 10N). However, the use of (R,S)-JQAD1 to degrade EP300, rather than combined catalytic inhibition of both EP300 and CBP, resulted in induction of a subG1 peak, consistent with apoptotic cell death (FIG. 3H; FIG. 10I). Thus, while A485 slows cell growth by G1 cell cycle arrest, (R,S)-JQAD1 has unique effects by inducing apoptotic cell death.

### JQAD1 Caused Dissociation of MYCN Protein from Chromatin

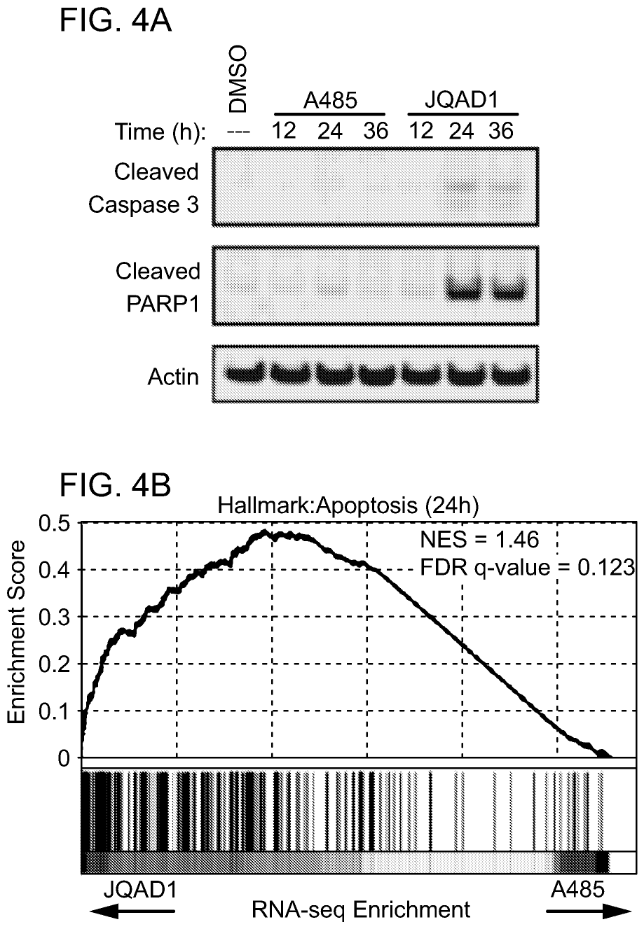

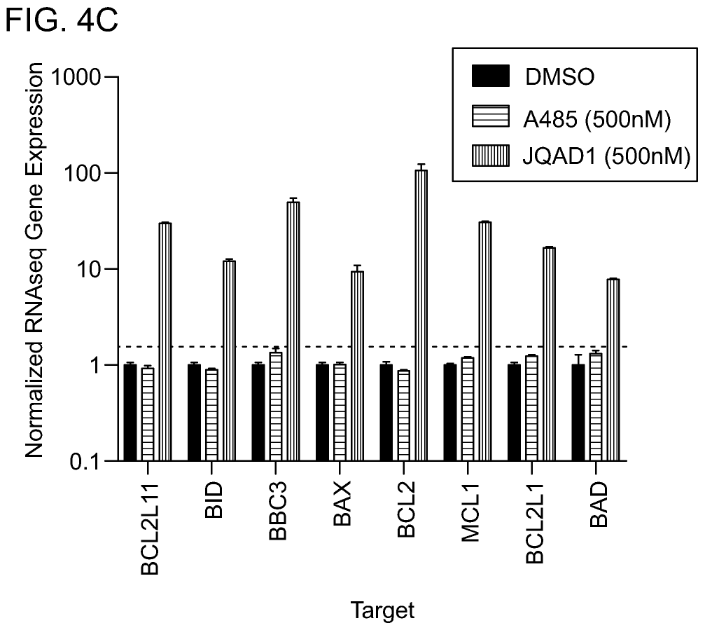

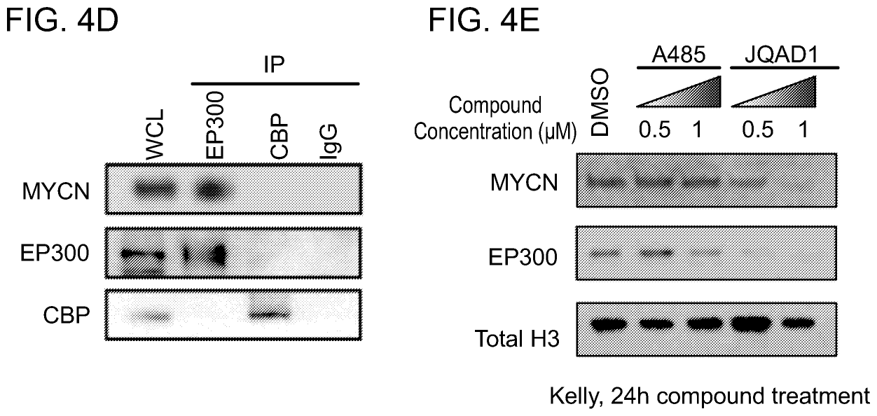

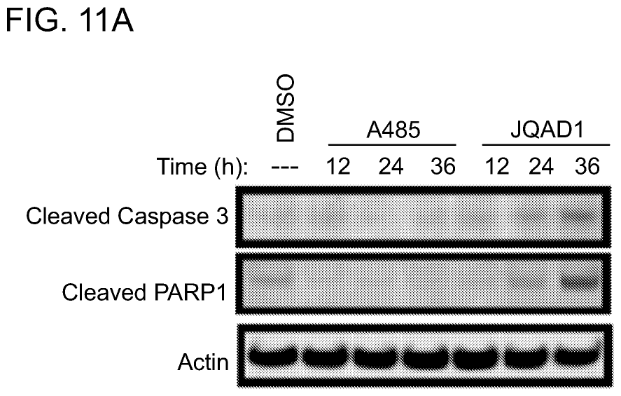

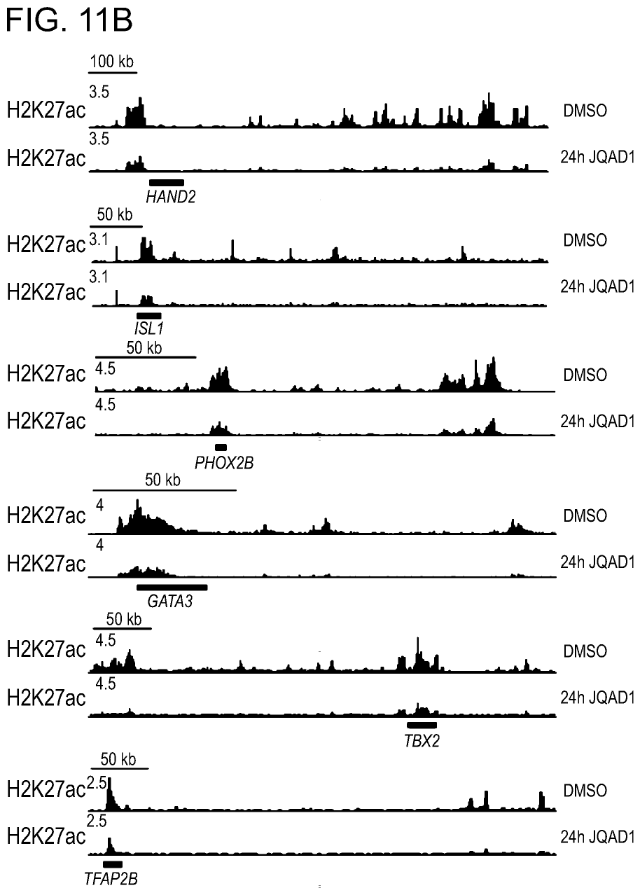

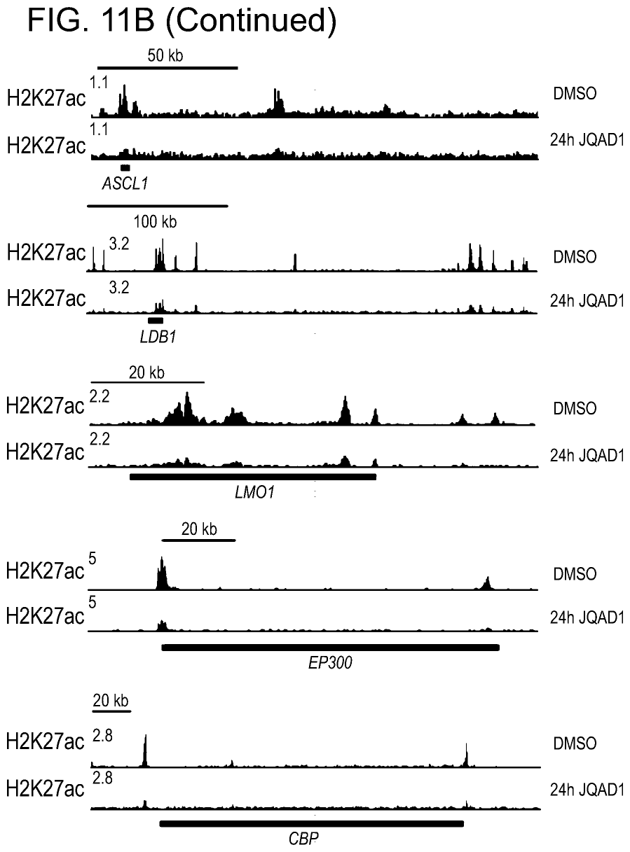

One key difference between the acute effects of A485 and JQAD1 treatment is that JQAD1 induced apoptosis consistent with the kinetics of loss of EP300, while A485 treatment resulted in G1 cell cycle arrest (FIG. 3G-FIG. 3I, FIG. 10H-FIG. 10N). To further demonstrate this difference in the induction of apoptosis by these two drugs, Kelly and NGP cells were treated for 12-36 h with equal concentrations of A485 or JQAD1 prior to protein extraction to analyze effects on apoptosis. Treatment of both cell lines with JQAD1 resulted in induction of cellular apoptosis marked by cleavage of caspase-3 and PARP1. In contrast, treatment with A485 had little effect on these parameters (FIG. 4A, FIG. 11A). To evaluate the mechanism underlying this difference in response, Kelly NB cells were treated with equivalent concentrations of DMSO, JQAD1 or A485 for 24 h prior to extraction of total RNA. RNA samples were then normalized with an exogenous spike-in RNA and used for RNA-seq analysis. RNA-seq results for JQAD1 and A485 treated samples were then compared by gene set enrichment (GSEA) analysis. Consistent with our DNA flow cytometry studies, GSEA analysis of the hallmark gene sets demonstrated enrichment of the apoptosis hallmark gene set in JQAD1 treated cells, compared with A485 treated cells (FIG. 4B). Furthermore, JQAD1-treated cells exhibited upregulation of the proapoptotic BH3-only effectors B-cell lymphoma 2 (Bcl-2)-like protein 11 (BIM), BH3 interacting-domain death agonist (BID), and p53-upregulated modulator of apoptosis (PUMA) together with the proapoptotic mediator BCL2 associated X protein (BAX) and its inhibitors BCL2 and myeloid cell leukemia 1(MCL1), while transcript levels for each of these mRNAs were unaffected in A485 treated cells (FIG. 4C). This suggests that these mRNA transcripts are upregulated through CBP, as JQAD1 specifically affected EP300, while A485 inhibited transcriptional activation by both HAT proteins.

One mechanism by which NB cells repress apoptosis is through high level expression and transcriptional activity of the MYCN oncoprotein, sometimes referred to as “oncogene addiction” (reviewed in Gabay et al. Cold Spring Harb. Perspect. Med. 4:a014241 (2014); Huang and Weiss, Cold Spring Harb Perspect Med. 3:a014415 (2013)). Further, EP300 and CBP are known to regulate the MYCN family member c-MYC protein by protein-protein interaction (Faiola et al. Mol. Cell Biol. 25:10220-34 (2005); Vervoorts et al. EMBO Rep. 4:484-90 (2003); Zhang et al. Biochem. Biophys. Res. Commun. 336:274-80 (2005)). Thus, it was hypothesized that a similar physical interaction between MYCN and EP300 might exist, resulting in stabilization of MYCN expression. Therefore, co-immunoprecipitation assays were performed with antibodies targeting endogenous EP300 or CBP in Kelly NB cells. Immunoprecipitation of protein from Kelly nuclear lysates with anti-EP300 antibodies, followed by western blotting, demonstrated pronounced association with MYCN protein. In contrast, immunoprecipitation of CBP, like IgG controls, did not reveal any association with MYCN proteins (FIG. 4D). Thus, in Kelly NB cells, EP300, but not CBP, physically interacts with the MYCN oncoprotein. To evaluate the functional significance of this interaction, Kelly NB cells we treated with DMSO or a variety of concentrations of A485 or JQAD1 for 24 h, and then isolated chromatin protein extracts to evaluate the presence of MYCN associated with chromatin. Kelly cells treated with A485 did not show MYCN loss from chromatin extracts up to 1.0 μM (FIG. 4E). By contrast, cells treated with JQAD1 to degrade EP300 showed loss of both EP300 and chromatin-bound MYCN proteins (FIG. 4E). These data support the interpretation that EP300 degradation by JQAD1 results in loss of chromatin-bound MYCN while inhibition of EP300 HAT activity does not affect chromatin localization. Thus, a physical interaction between EP300 and MYCN, separate from HAT enzymatic activity, maintains MYCN on chromatin, where it is necessary to promote cell growth and repress apoptosis in NB cells.

### JQAD1 Caused Loss of H3K27Ac Enriched at Super-Enhancers

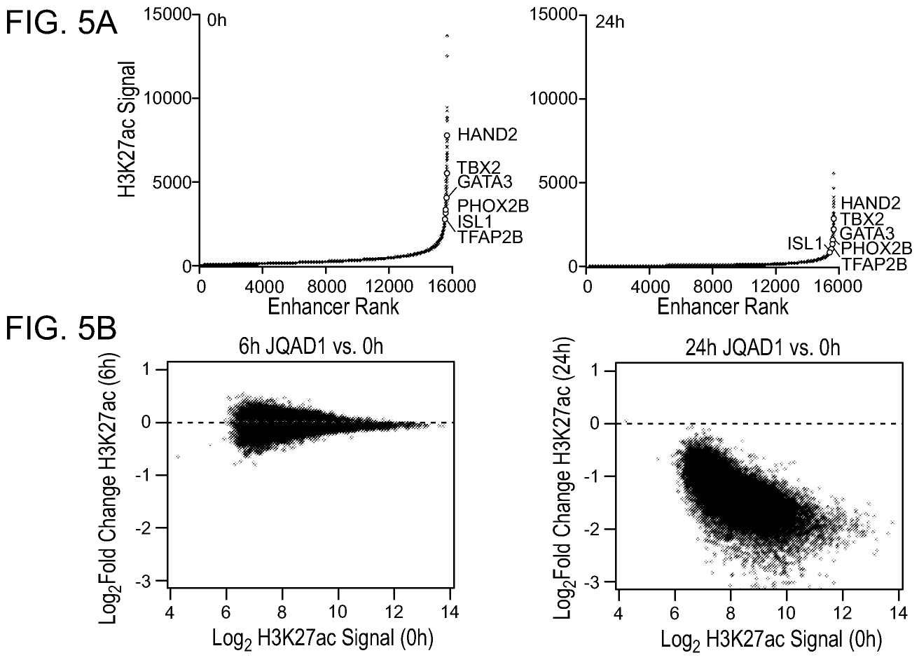

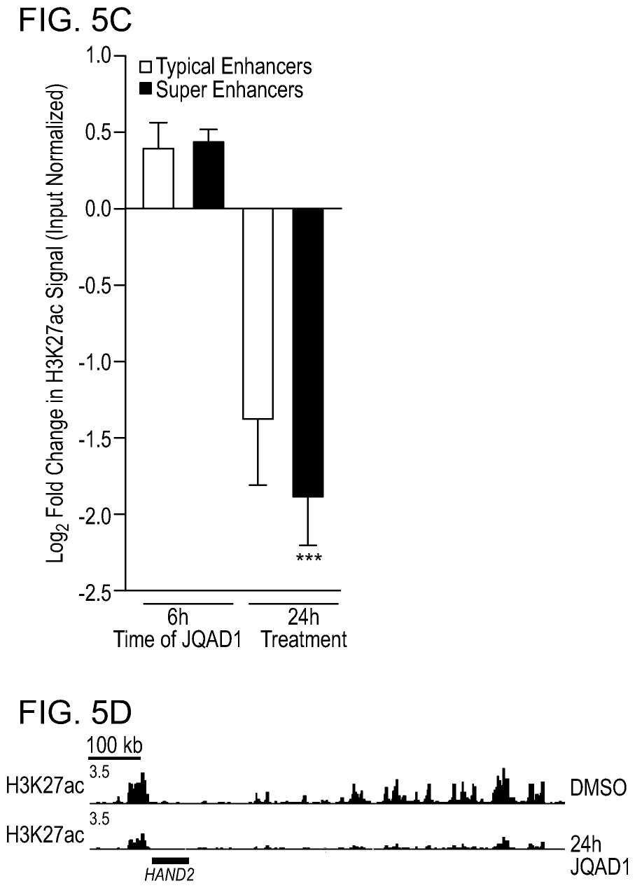

Since JQAD1 selectively degraded EP300 with minimal effects on CBP until 48 h in NB cell lines, JQAD1 was used to assess the effects of EP300 loss on genome-wide H3K27ac modifications. To determine these effects, ChIP-seq was performed with antibodies recognizing H3K27ac in Kelly NB cells over a time course from 0 to 24 h after exposure to (R,S)-JQAD1. These samples were externally normalized using spike-in Drosophila melanogaster chromatin. Comparison of all H3K27ac marked sites to untreated samples demonstrated approximately 2-fold global suppression of all enhancers by 24 h of treatment, at a time when EP300 was degraded and CBP was retained (FIG. 5A). While comparison of H3K27ac signal at earlier time points (6 h) to 0 h controls demonstrated no consistent change in acetylation, by 24 h of treatment, there was general loss of H3K27ac signal genome-wide, which was most pronounced at densely acetylated super-enhancers, including those regulating the core-regulatory circuitry. (FIG. 5B-FIG. 5D, FIG. 11B). These data indicate that super-enhancer loci in Kelly cells are regulated predominantly by EP300 and not CBP, because at this time point, EP300 was degraded without effects on the levels of CBP protein expression (FIG. 5D, FIG. 11B).

### JQAD1 was Effective with Limited Toxicity In Vivo

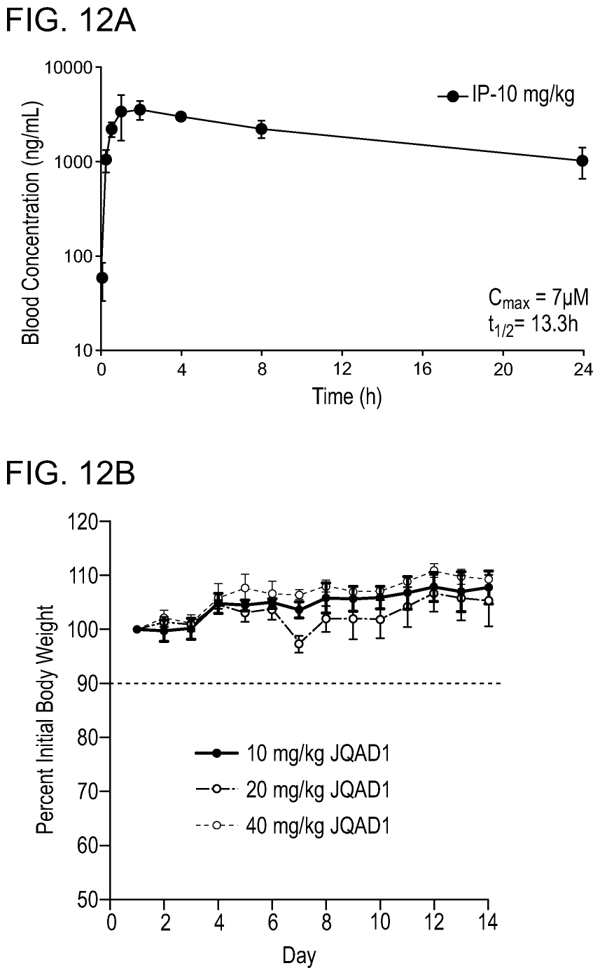

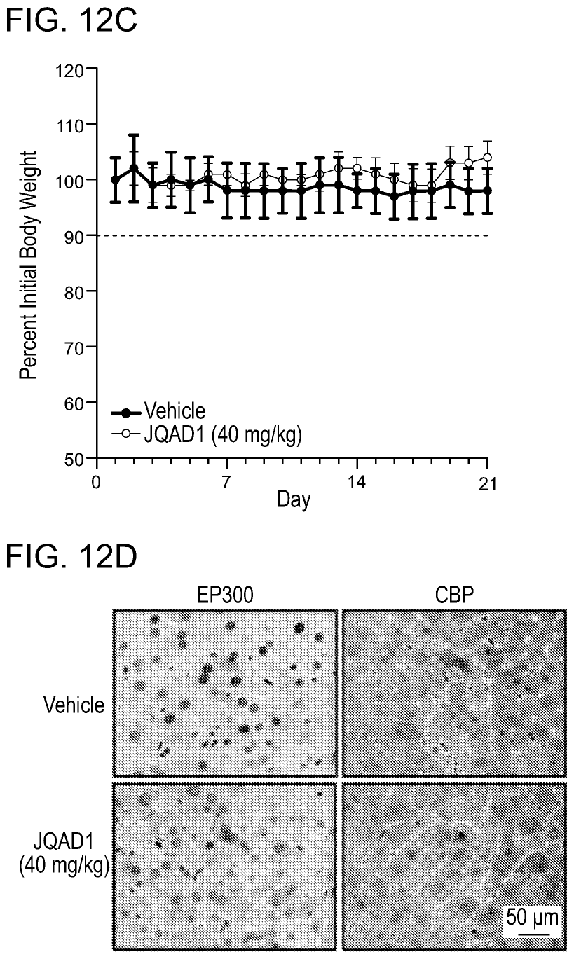

Because some CRBN-based PROTAC® agents have been shown to cause target protein degradation in vivo (reviewed in Burslem and Crews, Cell 181:102-14 (2020)), whether JQAD1 would actively degrade EP300 in vivo in human neuroblastoma xenograft models was investigated. First, pharmacokinetic analysis after a single intraperitoneal (IP) dose of JQAD1 at 10 mg/Kg was performed to identify the half-life and maximum serum concentration of the compound. After 10 mg/Kg intraperitoneal dosage, JQAD1 had a half-life of 13.3 (+/−3.37 SD) h in murine serum with a Cₘₐₓ of 7 μM (FIG. 12A), which is well above the IC₅₀ of human neuroblastoma cells in vitro (FIG. 8A-FIG. 8C). The maximum tolerated dose (MTD) in murine models was then investigated. Daily IP injection of JQAD1 at increasing doses in CD1 mice was performed. Daily IP treatment with JQAD1 was well tolerated with no signs of animal weight loss (FIG. 12B). Notably, with doses of JQAD1 higher than 40 mg/Kg, precipitation of the compound was observed in the peritoneal cavity. Thus, 40 mg/Kg of JQAD1 was determined to be the maximal dosage for single-dose IP administration without evident toxicity in the mouse.

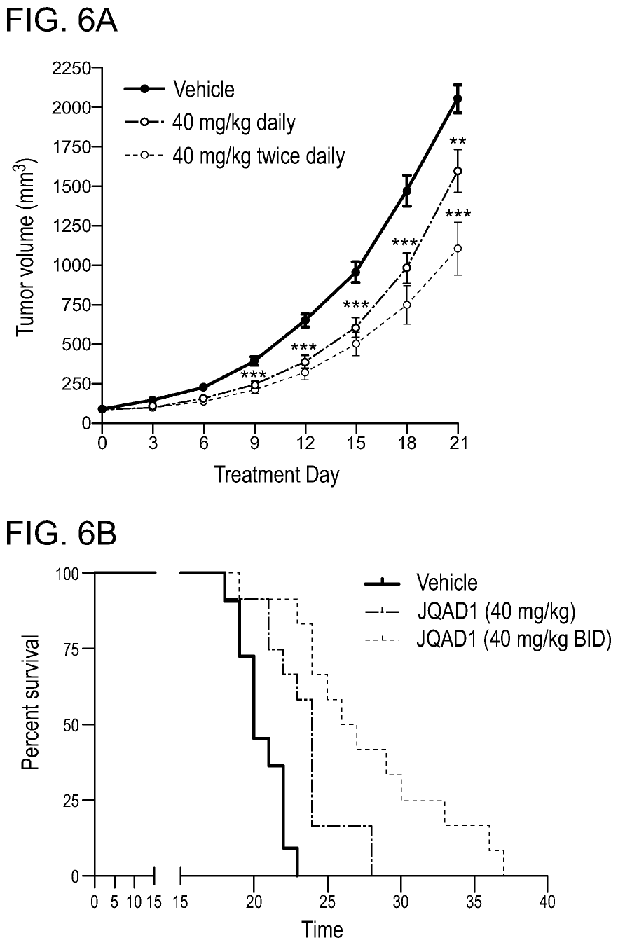

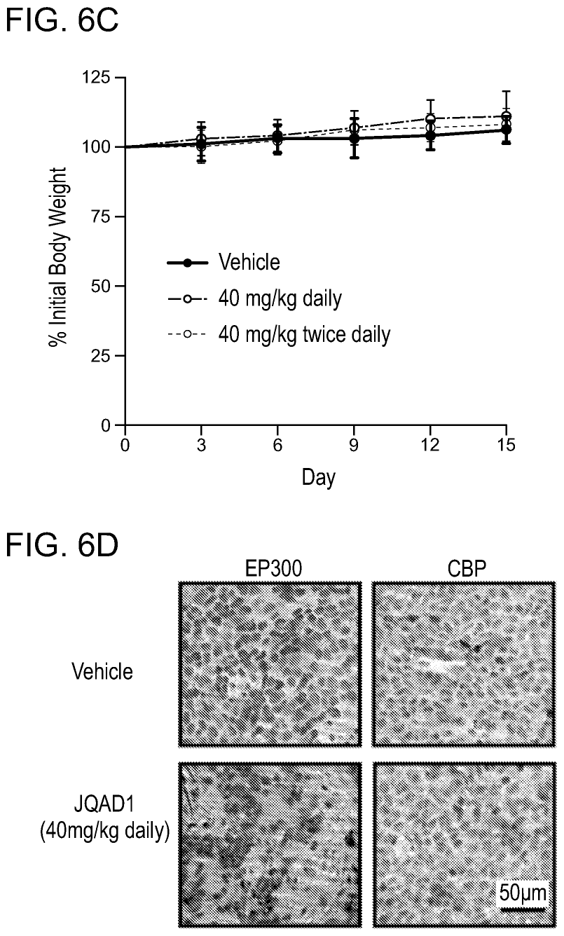

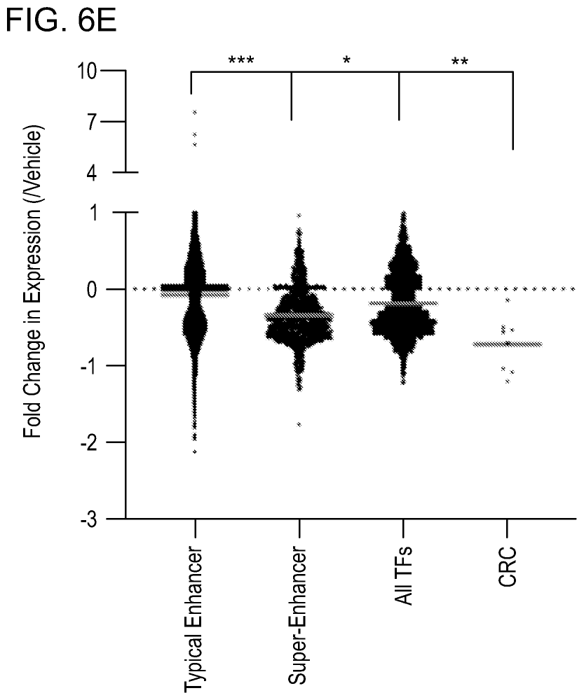

Next, subcutaneous xenografts of Kelly cells into the flanks of NOD scid gamma (NSG™) mice was established, and the mice were treated with either vehicle control or JQAD1 at 40 mg/Kg IP once or twice daily (FIG. 6A). JQAD1 treatment twice daily, and to a lesser extent, once daily, both caused a suppression of xenograft tumor growth by day 3 of treatment (p<0.05 by two-way ANOVA with post-hoc Tukey tests for tumor size, p<0.05 for suppressed growth rates in JQAD1 daily or twice daily treated tumors by mixed-effects analysis with post-hoc Tukey's multiple comparisons test), and prolongation of survival (log-rank test p<0.0001 each for once and twice daily dosing compared with vehicle control) (FIG. 6A-FIG. 6B). The effects of JQAD1 treatment on animal weight was also monitored, and body weight was maintained over 14 days of treatment prior to when control animals began to require sacrifice due to tumor burden (FIG. 6C). In a separate experiment, NSG™ mice were again xenografted with Kelly cells and treated with vehicle control or JQAD1 once daily at 40 mg/Kg for 10 days. Animals were then sacrificed, and the tumor was extracted. Tumor material was fixed for immunohistochemistry (IHC) and processed into single cells, prior to External RNA Controls Consortium (ERCC)-controlled RNA-seq analysis (FIG. 6D-FIG. 6E). Tumors recovered from animals treated with JQAD1 displayed a loss of EP300 immunostaining compared with vehicle control tumors, while CBP immunostaining was retained (FIG. 6D). Further, RNA expression profiles of tumor cells from mice treated with JQAD1, compared with vehicle control, demonstrated preferential downregulation of genes regulated by super-enhancers compared with those regulated by typical enhancers (FIG. 6E, p<0.0001).

Human CRBN differs from mouse at a key residue, CRBN^{Val388} compared to Crbn^{Ile391} in the mouse, which is important for binding, ubiquitinating and degrading key substrates including spalt-like transcription factor 4 (SALL4), a member of the spalt-like family of developmental transcription factors (Donovan et al. Elife 7:e38430 (2018); Fink et al. Blood 132:1535-44 (2018)). Thus, to assess the potential activity and toxic effects of JQAD1 more rigorously on murine tissues, JQAD1 was administered at 40 mg/Kg IP daily for 21 days to Balb/c Crbn^{ILE391VAL} humanized knockin mice (Fink et al. Blood 132:1535-44 (2018)). JQAD1 at this dosage was well tolerated, with no effects on grooming, behavior, weight, peripheral blood counts, liver function tests or creatinine measurements performed after 14 days of treatment (Table 1, FIG. 12C). After 14 days of treatment, three mice per each treatment group were sacrificed, and skin, brain, heart, lung, liver, spleen, kidney, pancreas, small intestine, colon, adrenal gland, and bladder were extracted and processed for pathologic analysis. Tissues were evaluated by an independently blinded pathologist, by hematoxylin and eosin staining for evidence of toxicity with tissue architectural changes. This revealed no gross changes in tissue architecture or immune infiltrate, consistent with a lack of toxicity. To establish whether JQAD1 was selective in degrading EP300 and not CBP in vivo, immunohistochemistry against EP300 and CBP was performed on liver tissue from Balb/c Crbn^{ILE391VAL} mice treated with vehicle control or JQAD1. This analysis demonstrated that JQAD1-treated animals had reduced EP300 protein expression levels in the liver cell nuclei compared with vehicle treated controls (FIG. 12D). Consistent with the hypothesis that CBP could partially compensate for loss of EP300, JQAD1-treated animals displayed no histologic or biochemical evidence of toxicity in the liver, and despite a loss of EP300 in vivo, CBP remained detectable by immunohistochemistry (FIG. 12D).

TABLE 1
JQAD1 had limited toxicity in vivo.

| | | | JQAD1 | | Normal | |
|---|---|---|---|---|---|---|
| Parameter | Vehicle | | (40 mg/Kg) | | Range | p |
| WBC (K/μL) | 3.86 | (2.9-4.18) | 3.46 | (1.36-6.56) | 1.8-10.7 | NS |
| Neutrophils | 1.37 | (0.89-1.9) | 1.2 | (0.39-3.01) | 0.1-2.4 | NS |
| Lymphocytes | 1.78 | (1.35- | 1.80 | (0.86-2.47) | 0.9-9.3 | NS |
| Monocytes (K/μL) | 0.43 | (0.27- | 0.35 | (0.09-0.71) | 0.0-0.4 | NS |
| Eosinophils | 0.12 | (0.06- | 0.10 | (0.01-0.3) | 0.0-0.2 | NS |
| Basophils (K/μL) | 0.03 | (0.01- | 0.02 | (0-0.07) | 0.0-0.2 | NS |
| Hemoglobin | 14.8 | (12.2-17) | 13.35 | (11.4-15.3) | 11.0-15.1 | NS |
| Hematocrit (%) | 46.8 | (40-52.8) | 41.2 | (33-50.3) | 35.1-45.4 | NS |
| Platelet (K/μL) | 904 | (351- | 528 | (192-1071) | 592-2972 | NS |
| Creatinine | 0.16 | (0.15- | 0.17 | (0.15-0.22) | NR | NS |
| Albumin | 3.8 | (2.7-4.6) | 3.2 | (2.8-4.0) | NR | NS |
| AST | 57.5 | (27-148) | 37 | (23-64) | NR | NS |
| ALT | 92.5 | (26-184) | 54 | (47-198) | NR | NS |
| ALP | 24 | (10-38) | 51 | (28-62) | NR | NS |
| GGTP | 5 | (5-46) | 5 | (5) | NR | NS |

EP300, but less commonly CBP, was identified as a dependency in neuroblastoma, along with MYCN and each of the members of the adrenergic CRC (Durbin et al. Nat. Genet. 50:1240-6 (2018)). Since EP300 catalyzes the H3K27ac mark, it was hypothesized that EP300 might preferentially be responsible for the high levels of expression of CRC master transcription factors. Because JQAD1 preferentially degraded EP300, the HAT that primarily catalyzes H3K27ac seen at super-enhancers, it was reasoned that treatment with JQAD1 might have major effects of the expression levels of genes in the CRC. Therefore, the effects of JQAD1 given daily for 14 days on the expression levels of several different classes of mRNAs were compared, including those regulated by typical enhancers, super-enhancers, and all TFs as well as TFs that encoded members of the CRC (FIG. 6E). This comparison revealed that the CRC genes, along with MYCN, were among the most downregulated genes in tumors treated with JQAD1, compared to genes in the other categories. These results demonstrate that JQAD1 treatment drastically downregulated the expression of the very important subset of genes that encode transcription factors comprising the CRC, including MYCN, which is a dominant oncogene in neuroblastoma cells and one that neuroblastoma cells are dependent on for growth and survival (Durbin et al. Nat. Genet. 50:1240-6 (2018); Pugh et al. Nat. Genet. 45:279-84 2013; Zeid et al. Nat Genet 50:515-23(2018)).

### JQAD1 had Broad Anti-Neoplastic Activity Across Cancer Cell Lines

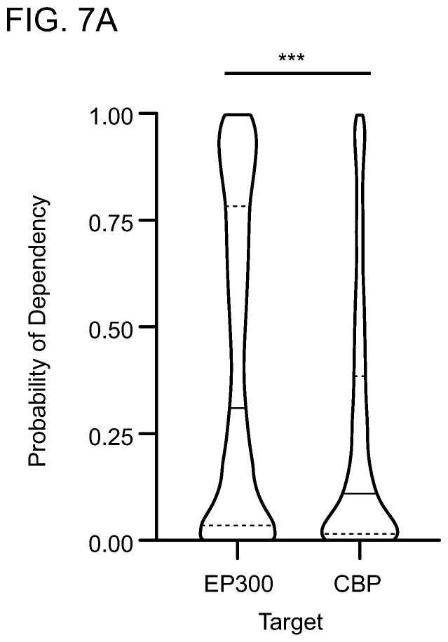

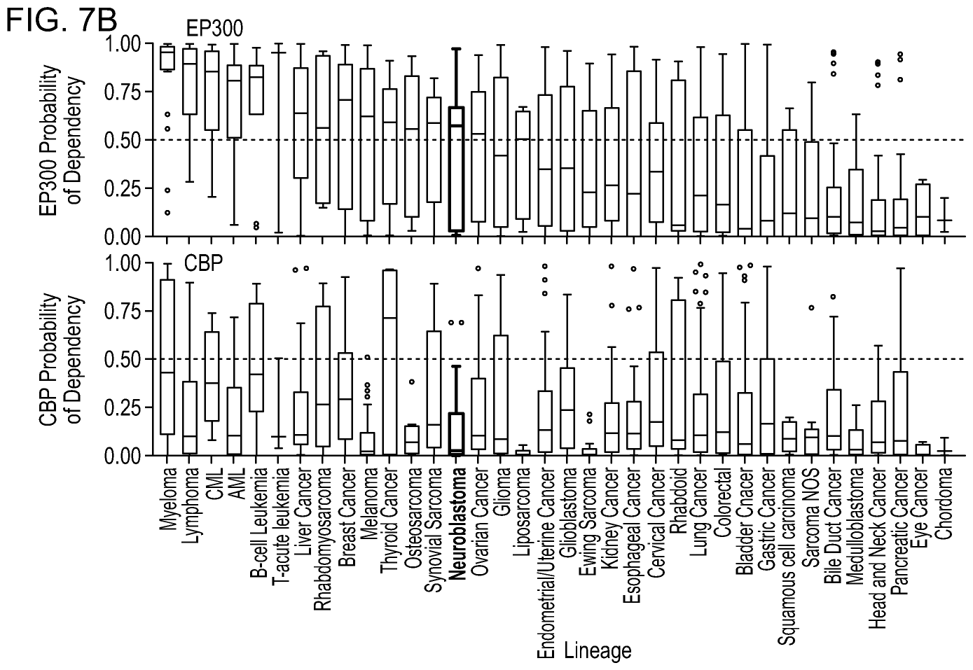

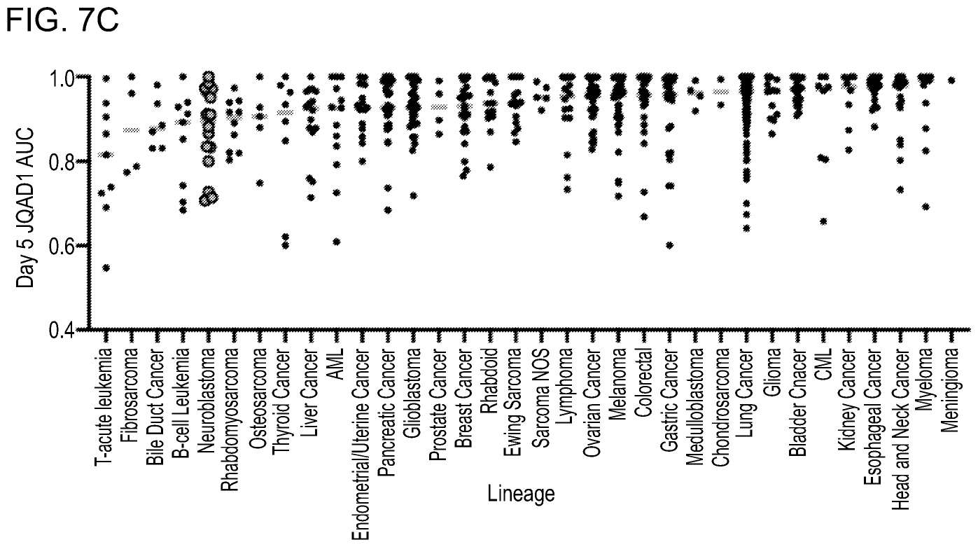

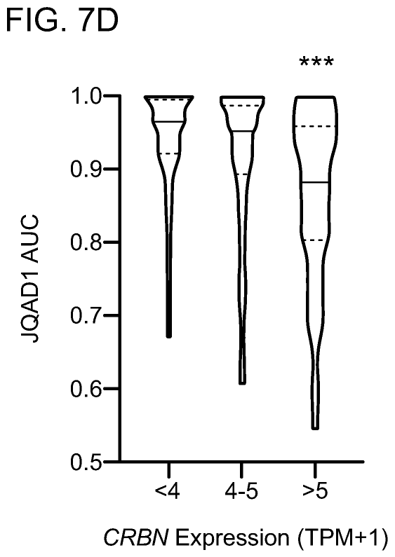

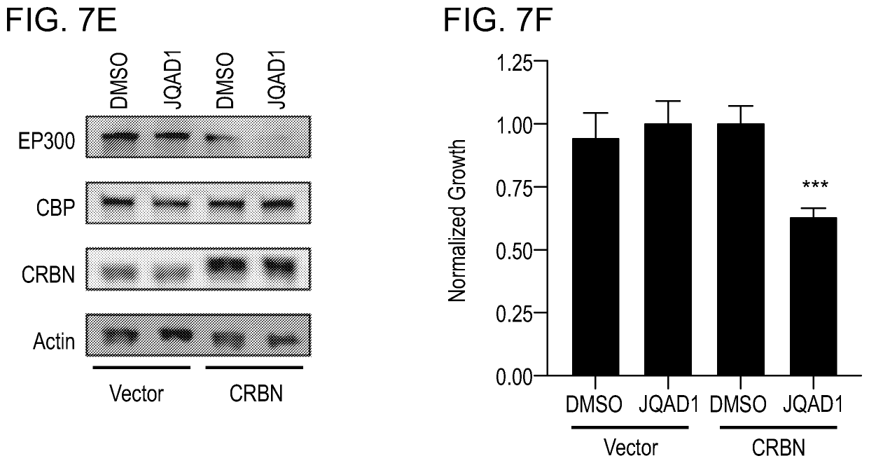

Epigenetic and enhancer-mediated control of gene expression is required for normal cellular and tissue developmental processes and is dysregulated in different cancer subtypes (reviewed in Bradner et al. Cancer. Cell 168:629-43 (2017); Wimalasena et al. Mol Cell 78:1086-95 (2020)). In neuroblastoma cells, EP300 is a dominant controller of H3K27ac, signifying active promoters and enhancers, in addition to transcriptional activity. Therefore, it was hypothesized that there may be a preferential reliance on EP300 or CBP across other cancer subtypes as well. Thus, the relative dependence of all available cell lines on EP300 or CBP was examined using the DepMap genome-scale CRISPR-Cas9 loss-of-function screening dataset (Meyers et al. Nat. Genet. 49:1779-84 (2017)). Comparison of the probability of dependency on EP300 and CBP across a total of 757 human cancer cell lines, representing 36 distinct tumor lineages, demonstrated a higher probability of dependency on EP300 than CBP across many cancer cell lines (p<0.0001, FIG. 7A). Thus, all cell lines in DepMap were stratified by tumor lineage, and the probability of dependency on EP300 and CBP in each lineage was examined. By this analysis, many tumor lineages displayed an enhanced probability of dependency on EP300 compared with CBP (FIG. 7B). Few tumor cell lineages, notably thyroid, pancreatic, and cervical carcinomas displayed enhanced dependency on CBP compared to EP300 (FIG. 7B).

Because the probability of dependency on EP300 was higher for many tumor lineages than that of CBP, whether JQAD1 would display antineoplastic effects across multiple tumor lineages was assessed. The response to JQAD1 in a pooled and barcoded 5-day cell viability PRISM screen conducted at the Broad Institute with 557 cancer cell lines was analyzed (FIG. 7C) (Corsello et al. Nat. Cancer 1:235-48 (2020)). These results demonstrate that JQAD1 treatment had antineoplastic activity across multiple tumor lineages, many of which display enhanced dependency on EP300 compared with CBP. Within the majority of tumor lineages, some example cell lines displayed growth inhibition with JQAD1 treatment (area under the curve (AUC)<0.85) (FIG. 7C). Since cell lines from multiple lineages displayed growth suppression with JQAD1 treatment, whether predictors of JQAD1 activity could be determined was assessed. Accordingly, an analysis of RNA expression profiles of all cell lines treated with JQAD1 was performed. Consistent with its mechanism of action, higher expression levels of CRBN were correlated with higher JQAD1-mediated antineoplastic activity as reflected by a lower AUC measurement of JQAD1 dose-response (FIG. 7D). This indicates that JQAD1 activity was at least partially determined by CRBN expression levels, which is consistent with the requirement by JQAD1 for CRBN to target EP300 for degradation.

To further investigate this requirement, it was hypothesized that increasing the expression levels of CRBN in JQAD1-resistant cells may result in restoration of sensitivity. Thus, the response of BE2C neuroblastoma cells, which display lower CRBN protein expression, to JQAD1 was examined (FIG. 10C). BE2C cells with stable overexpression of CRBN (BE2C-CRBN) or, as a control, zsGreen (BE2C-zsGreen) were established, and then cells were treated with either DMSO or JQAD1 (FIG. 7E). EP300 was degraded within 24 h of treatment with JQAD1 in BE2C-CRBN cells, while expression of EP300 in control cells was unaffected (FIG. 7E). Concordant with these results, the growth of BE2C-CRBN cells was suppressed by JQAD1 treatment, while untreated BE2C-CRBN cells grew at similar rates as BE2C-zsGreen cells treated with DMSO or JQAD1 (FIG. 7F). Thus, CRBN overexpression in JQAD1-insensitive BE2C cells was sufficient to restore sensitivity to the compound, JQAD1. These data underscore that two important considerations for using degraders across distinct cell models include both individual cell line dependency on the PROTAC® target, in addition to expression levels of key components of the PROTAC® machinery, such as CRBN.

In summary, these data indicate that cancer cells in addition to neuroblastoma display enhanced dependency on EP300, compared to CBP, and that JQAD1 represents a potential method to capitalize on this enhanced dependency, especially in individual tumors with elevated CRBN expression levels.

The basis for selective dependency in most childhood neuroblastomas on EP300 and not on CBP is demonstrated herein. It is also demonstrated that, in the adrenergic subtype of neuroblastoma, the AP2 family transcription factor TFAP2β is a key member of the core-regulatory circuitry that co-binds genome-wide along with the remainder of the CRC factors. Core-regulatory circuitries are lineage-defining autoregulatory transcription factor networks that establish the transcriptional landscapes of different types of cells (Boyer et al. Cell 122:947-56 (2005); Durbin et al. Nat. Genet. 50:1240-6 (2018); Saint-André et al. Genome Res. 26:385-96 (2016); Sanda et al. Cancer Cell 22:209-21 (2012); Wang et al. Nat. Commun. 10:5622 (2019)). EP300 and CBP do not recognize sequence-specific DNA motifs, and thus depend on transcription factors to localize them to their target enhancers. Importantly, TFAP2β specifically binds EP300, but not CBP, establishing the basis for dependency on EP300. TFAP2β, therefore, specifically associates with EP300 at the enhancers that form the extended regulatory network of the adrenergic NB CRC across the genome, including the network of genes that establish the malignant cell state in this subtype of neuroblastoma. Thus, loss of TFAP2β results in loss of the H3K27ac mark on CRC associated super-enhancers catalyzed by EP300 in neuroblastoma cells, thereby identifying TFAP2β as a dominant mediator of EP300 localization to critical super-enhancers. This mechanism results in direct regulation of lineage-specifying and oncogenic loci in neuroblastoma through recruitment of EP300 by physical interaction with the novel CRC transcription factor TFAP2β. This function cannot be accomplished by CBP, because it does not physically interact with TFAP2β, or indeed with other transcription factors of the adrenergic CRC. In addition to transcription factors, other elements of core-regulatory circuitries including enhancer RNAs and linker proteins such as LDB1 and LMO1 are integral components of this regulatory complex (Sanda et al. Cancer Cell 22:209-21 (2012); Suzuki et al. Cell 168:1000-14 (2017); Wang et al. Nat. Commun. 10:5622 (2019)). With evidence that coactivator proteins are found at genomic loci bound by CRC transcription factors and that loss of EP300 results in enhanced loss of CRC factor expression compared with other transcription factors in vivo, it was posited that coactivator enzymes such as EP300 are critical for the high levels of expression that define genes of the CRC extended regulatory network, and that lineage- and tumor-specific CRC factors such as TFAP2β in neuroblastoma play a novel role in the CRC complex, being required for recruiting EP300 to establish the malignant cell state (Sabari et al. Science 361: eaar3958 (2018)).

It has been demonstrated that the activity of the CRC through its target enhancers is required for cell growth and viability in adrenergic neuroblastoma (Durbin et al. Nat. Genet. 50:1240-6 (2018); Wang et al. Nat. Commun. 10:5622 (2019)). Thus, it is not surprising that EP300 is a major dependency in neuroblastoma, while CBP is not a dependency in most NB cell lines, presumably because it is not required to maintain high levels of expression of the network of genes driven by the CRC in this disease.

There is a striking enrichment for dependency on EP300 compared to CBP in various cancer subtypes, highlighting the hypothesis that these two paralogous genes may play context-dependent and distinct roles in regulating cancer cell survival. As a result, selective targeting of EP300 in different types of cancer cell lines that are dependent of EP300 may be effective for eliciting anti-tumor activity, with reduced toxicity because CBP is still active in normal cells and may be able in most normal cells to compensate for the loss of EP300. This attractive hypothesis has been hard to test, because of significant homology between these two proteins, which has prevented pharmacologic strategies to preferentially target one of these enzymes, while sparing activity of the other.

PROTAC® JQAD1, which relies on the binding activity of A485 and is selective in its ability to degrade EP300 compared to CBP, is described herein. This observation stands in marked contrast to the more promiscuous acetyltransferase inhibitory activity of A485 against both EP300 and CBP. PROTAC® agents, synthesized from bait molecules with binding to several closely related proteins, in some cases display substrate specificity, such as with bromodomain-containing protein 4 (BRD4) and p38 degraders (reviewed in Burslem and Crews, Cell 181:102-14 (2020)). The mechanism of this selectively is likely to be related to three-dimensional interactions between chimeric degrader compounds and the E3 ligase complex, mediated by the three-dimensional structure of the target protein and E3 ligase receptor. Due to the size and lack of solubility of full-length EP300 and CBP proteins, full-length crystal structures have not been resolved. However, Biotin-JQAD1 forms a ternary complex with EP300 and CRBN, which does not contain CBP. Thus, in contrast to A485, which has equivalent activity against EP300 and CBP, JQAD1 bound more avidly to EP300 in biochemical assays.

JQAD1 has several intriguing properties: i) It demonstrated selectivity for EP300 relative to CBP in multiple neuroblastoma cell lines; ii) It had higher potency than the parental inhibitor in some cell lines; and iii) It was useful for degradation of EP300 with limited effects on CBP and limited toxicities in vivo. EP300 was degraded by JQAD1 in vivo in normal murine tissues that express humanized CRBN, however, CBP staining was only minimally affected in these tissues. Further, these tissues display normal architecture. These data support the hypothesis that CBP compensates for the loss of EP300 in some normal tissues. Accordingly, no toxicity was observed in mice treated with twice daily with 40 mg/Kg JQAD1 IP for 14 days after profiling blood counts, liver and kidney function tests, weight, and grooming. Thus, it was hypothesized that CBP-mediated activities are able to compensate for loss of EP300 at least partially in untransformed cells.

Experiments using JQAD1 also permitted the identification of a skewed activity toward loss of H3K27ac signal prior to effects of expression of genes that form the extended regulatory network of the CRC. JQAD1 caused selective degradation of full-length EP300 compared with the catalytic inhibition of EP300 and CBP by A485. This indicates that loss of full-length EP300 causes induction of apoptosis in neuroblastoma cells compared with catalytic inhibition. In neuroblastoma, EP300 physically interacts with the dominant tumor oncoprotein MYCN, controlling its localization to chromatin. Thus, degradation of EP300 results in loss of this binding activity, which then leads to disassociation of MYCN from chromatin. Prior evidence indicates that MYCN, and indeed other MYC proteins, engage chromatin widely to cause enhancer invasion and are independently required to repress apoptosis in neuroblastoma cells (Huang and Weiss, Cold Spring Harb Perspect Med. 3:a014415 (2013); Zeid et al. Nat Genet 50, 515-23 (2018)). Thus, these data implicate a new mechanism by which MYCN is maintained in a chromatin-associated state through physical interactions with EP300, which thereby facilitates enhancer invasion and MYCN-mediated enhancement of CRC-based oncogenic transcription.

Thus, distinct roles for EP300 and CBP in the regulation of cell growth in high-risk pediatric neuroblastoma are described herein. These findings were similarly identified in a variety of other tumor types, indicating that enhanced dependency on EP300 is a common finding in human cancers. EP300, but not CBP, is required for regulation of H3K27ac and the gene expression landscape of a subset of high-risk neuroblastoma. This function is performed due to interaction between EP300 and the new CRC transcription factor TFAP2β that mediates EP300 binding to enhancers and promoters associated with the CRC. In doing so, TFAP2β and EP300 collaborate to determine gene expression patterns in the adrenergic subtype of high-risk neuroblastoma. PROTAC® JQAD1was generated to capitalize on these findings. Importantly, loss of EP300 results in disassociation of the dominant neuroblastoma oncoprotein MYCN from chromatin, resulting in a loss of enhancer invasion, suppression of CRC-based transcription and apoptosis. These data provided key insights into enhancer control in high-risk neuroblastoma and highlighted a new paradigm for chemical epigenetic control of gene enhancers and mRNA expression in high-risk neuroblastoma with implications for other types of human cancers.

### World Health Organization Criteria

The WHO Criteria for evaluating the effectiveness of anti-cancer agents on tumor shrinkage, developed in the 1970s by the International Union Against Cancer and the World Health Organization, represented the first generally agreed specific criteria for the codification of tumor response evaluation. These criteria were first published in 1981 (Miller et al. 1981 Clin. Cancer Res., 47:207-14). WHO Criteria proposed>50% tumor shrinkage for a Partial Response and >25% tumor increase for Progressive Disease.

### Response Evaluation Criteria in Solid Tumors (RECIST)

RECIST is a set of published rules that define when tumors in cancer patients improve (“respond”), stay the same (“stabilize”), or worsen (“progress”) during treatment (Eisenhauer et al. 2009 European Journal of Cancer, 45:228-247). Only patients with measurably disease at baseline should be included in protocols where objective tumor response is the primary endpoint.

The response criteria for evaluation of target lesions are as follows:
Complete Response (CR): Disappearance of all target lesions.
Partial Response (PR): At least a 30% decrease in the sum of the longest diameter (LD) of target lesions, taking as reference the baseline sum LD.
Stable Disease (SD): Neither sufficient shrinkage to qualify for PR nor sufficient increase to qualify for PD, taking as reference the smallest sum LD since the treatment started.
Progressive Disease (PD): At least a 20% increase in the sum of the LD of target lesions, taking as reference the smallest sum LD recorded since the treatment started or the appearance of one or more new lesions. The response criteria for evaluation of non-target lesions are as follows:
Complete Response (CR): Disappearance of all non-target lesions and normalization of tumor marker level.
Incomplete Response/Stable Disease (SD): Persistence of one or more non-target lesion(s) or/and maintenance of tumor marker level above the normal limits.
Progressive Disease (PD): Appearance of one or more new lesions and/or unequivocal progression of existing non-target lesions.

The response criteria for evaluation of best overall response are as follows. The best overall response is the best response recorded from the start of the treatment until disease progression/recurrence (taking as reference for PD the smallest measurements recorded since the treatment started). In general, the patient's best response assignment will depend on the achievement of both measurement and confirmation criteria.
Patients with a global deterioration of health status requiring discontinuation of treatment without objective evidence of disease progression at that time should be classified as having “symptomatic deterioration”. Every effort should be made to document the objective progression even after discontinuation of treatment.
In some circumstances, it may be difficult to distinguish residual disease from normal tissue. When the evaluation of complete response depends on this determination, it is recommended that the residual lesion be investigated (fine needle aspirate/biopsy) to confirm the complete response status.

### Immune-Related Response Criteria

The immune-related response criteria (irRC) are a set of published rules that define when tumors in cancer patients improve (“respond”), stay the same (“stabilize”), or worsen (“progress”) during treatment, where the compound being evaluated is an immuno-oncology drug. The Immune-Related Response Criteria, first published in 2009 (Wolchok et al. Clin. Cancer Res. 15:7412 (2009)), arose out of observations that immuno-oncology drugs would fail in clinical trials that measured responses using the WHO or RECIST Criteria, because these criteria could not account for the time gap in many patients between initial treatment and the apparent action of the immune system to reduce the tumor burden. The key driver in the development of the irRC was the observation that, in studies of various cancer therapies derived from the immune system such as cytokines and monoclonal antibodies, the looked-for Complete and Partial Responses as well as Stable Disease only occurred after an increase in tumor burden that the conventional RECIST Criteria would have dubbed “Progressive Disease”. RECIST failed to take account of the delay between dosing and an observed anti-tumor T cell response, so that otherwise ‘successful’ drugs - that is, drugs which ultimately prolonged life—failed in clinical trials.

The irRC are based on the WHO Criteria; however, the measurement of tumor burden and the assessment of immune-related response have been modified as set forth below.

### Measurement of Tumor Burden

In the irRC, tumor burden is measured by combining ‘index’ lesions with new lesions. Ordinarily, tumor burden would be measured with a limited number of ‘index’ lesions (that is, the largest identifiable lesions) at baseline, with new lesions identified at subsequent time points counting as ‘Progressive Disease’. In the irRC, by contrast, new lesions are a change in tumor burden. The irRC retained the bidirectional measurement of lesions that had originally been laid down in the WHO Criteria.

### Assessment of Immune-Related Response

In the irRC, an immune-related Complete Response (irCR) is the disappearance of all lesions, measured or unmeasured, and no new lesions; an immune-related Partial Response (irPR) is a 50% drop in tumor burden from baseline as defined by the irRC; and immune-related Progressive Disease (irPD) is a 25% increase in tumor burden from the lowest level recorded. Everything else is considered immune-related Stable Disease (irSD). Even if tumor burden is rising, the immune system is likely to “kick in” some months after first dosing and lead to an eventual decline in tumor burden for many patients. The 25% threshold accounts for this apparent delay.

### Gene Expression Profiling

In general, methods of gene expression profiling may be divided into two large groups: methods based on polynucleotide hybridization analysis and methods based on polynucleotide sequencing. Methods known in the art for the quantification of mRNA expression in a sample include northern blotting and in situ hybridization, RNAse protection assays, RNA-seq, and reverse transcription polymerase chain reaction (RT-PCR). Alternatively, antibodies are employed that recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). For example, RT-PCR is used to compare mRNA levels in different sample populations, in normal and tumor tissues, with or without drug treatment, to characterize patterns of gene expression (i.e., expression level), to discriminate between closely related mRNAs, and/or to analyze RNA structure.

In some cases, a first step in gene expression profiling by RT-PCR is the reverse transcription of the RNA template into cDNA, followed by amplification in a PCR reaction. For example, extracted RNA is reverse transcribed using a GeneAmp RNA PCR kit (Perkin Elmer, Calif, USA), following the manufacturer's instructions. The cDNA is then used as template in a subsequent PCR amplification and quantitative analysis using, for example, a TaqMan™ Respiratory Tract Microbiota® (Life Technologies™, Inc., Grand Island, N.Y.) assay.

Microarrays. Differential gene expression can also be identified or confirmed using a microarray technique. In these methods, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines and corresponding normal tissues or cell lines. Thus, RNA is isolated from a variety of primary tumors or tumor cell lines. If the source of mRNA is a primary tumor, mRNA is extracted from frozen or archived tissue samples.

In the microarray technique, PCR-amplified inserts of cDNA clones are applied to a substrate in a dense array. The microarrayed genes, immobilized on the microchip, are suitable for hybridization under stringent conditions.

In some cases, fluorescently labeled cDNA probes are generated through incorporation of fluorescent nucleotides by reverse transcription of RNA extracted from tissues of interest (e.g., leukemia tissue). Labeled cDNA probes applied to the chip hybridize with specificity to loci of DNA on the array. After washing to remove non-specifically bound probes, the chip is scanned by confocal laser microscopy or by another detection method, such as a charge-coupled device (CCD) camera. Quantification of hybridization of each arrayed element allows for assessment of corresponding mRNA abundance.

In some configurations, dual color fluorescence is used. With dual color fluorescence, separately labeled cDNA probes generated from two sources of RNA are hybridized pairwise to the array. The relative abundance of the transcripts from the two sources corresponding to each specified gene is thus determined simultaneously. In various configurations, the miniaturized scale of the hybridization can afford a convenient and rapid evaluation of the expression pattern for large numbers of genes. In various configurations, such methods can have sensitivity required to detect rare transcripts, which are expressed at fewer than 1000, fewer than 100, or fewer than 10 copies per cell. In various configurations, such methods can detect at least approximately two-fold differences in expression levels (Schena et al. Proc. Natl. Acad. Sci. USA 93:106-149 (1996)). In various configurations, microarray analysis is performed by commercially available equipment, following manufacturer's protocols, such as by using the Affymetrix GenChip technology, or Incyte's microarray technology.

RNA sequencing (RNA-seq), also called whole transcriptome shotgun sequencing (WTSS), is another technique to identify or confirm differential gene expression. RNA-seq uses next-generation sequencing (NGS) to reveal the presence and quantity of RNA in a biological sample at a given moment in time.

RNA-Seq is used to analyze the continually changing cellular transcriptome. See, e.g., Wang et al. Nat. Rev. Genet. 10:57-63 (2009). Specifically, RNA-Seq facilitates the ability to look at alternative gene spliced transcripts, post-transcriptional modifications, gene fusion, mutations/SNPs, and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries.

Prior to RNA-Seq, gene expression studies were done with hybridization-based microarrays. Issues with microarrays include cross-hybridization artifacts, poor quantification of lowly and highly expressed genes, and the need to know the sequence of interest. Because of these technical issues, transcriptomics transitioned to sequencing-based methods. These progressed from Sanger sequencing of Expressed Sequence Tag libraries to chemical tag-based methods (e.g., serial analysis of gene expression), and finally to the current technology, NGS of cDNA (notably RNA-Seq).

### Pharmaceutical Therapeutics

For therapeutic uses, the agents (e.g., JQAD1) described herein may be administered systemically, for example, formulated in a pharmaceutically-acceptable buffer such as physiological saline. Preferable routes of administration include, for example, subcutaneous, intravenous, intraperitoneal, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the agents to be administered varies depending upon the manner of administration, the age and body weight of the patient, and with the clinical symptoms of (e.g., NB). Generally, amounts will be in the range of those used for other agents used in the treatment of other diseases associated with EP300 dependency (e.g., cancer (e.g., NB)), although in certain instances lower amounts will be needed because of the increased specificity of the agents. For example, an agent is administered at a dosage that is cytotoxic to a neoplastic cell.

In one aspect, the disease or disorder is a cancer. In certain embodiments, the cancer is solid tumor, for example, neuroblastoma, rhabdomyosarcoma, melanoma, colon cancer, rectum cancer, stomach cancer, breast cancer, brain cancer, and pancreatic cancer. In certain embodiments the cancer is a hematologic cancer, for example, leukemia, myeloma, and lymphoma. In certain embodiments, the cancer is high-risk neuroblastoma. In some embodiments, the EP 300 dependent cancer is high-risk NB.

### Formulations

Human dosage amounts can initially be determined by extrapolating from the amount of the agent used in animal models, as a skilled artisan recognizes it is routine in the art to modify the dosage for humans compared to animal models. In certain embodiments, it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other cases, this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other aspects, it is envisaged that doses may be in the range of about 5 mg compound/Kg body to about 20 mg compound/Kg body. In other embodiments, the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

In some cases, the agent of the invention is administered at a dose that is lower than the human equivalent dosage (HED) of the no observed adverse effect level (NOAEL) over a period of three months, four months, six months, nine months, 1 year, 2 years, 3 years, 4 years or more. The NOAEL, as determined in animal studies, is useful in determining the maximum recommended starting dose for human clinical trials. For instance, the NOAELs can be extrapolated to determine human equivalent dosages. Typically, such extrapolations between species are conducted based on the doses that are normalized to body surface area (i.e., mg/m²). In specific embodiments, the NOAELs are determined in mice, hamsters, rats, ferrets, guinea pigs, rabbits, dogs, primates, primates (monkeys, marmosets, squirrel monkeys, baboons), micropigs or minipigs. For a discussion on the use of NOAELs and their extrapolation to determine human equivalent doses, see Guidance for Industry Estimating the Maximum Safe Starting Dose in Initial Clinical Trials for Therapeutics in Adult Healthy Volunteers, U.S. Department of Health and Human Services Food and Drug Administration Center for Drug Evaluation and Research (CDER), Pharmacology and Toxicology, July 2005.

The amount of an agent of the invention used in the prophylactic and/or therapeutic regimens which will be effective in the treatment of a hematopoietic cancer, or an autoimmune disease can be based on the currently prescribed dosage of the agent as well as assessed by methods disclosed herein and known in the art. The frequency and dosage will vary also according to factors specific for each patient depending on the specific agent administered, the severity of the cancerous condition, the route of administration, as well as age, body, weight, response, and the past medical history of the patient. For example, the dosage of an agent of the invention which will be effective in the treatment of cancer can be determined by administering the agent to an animal model such as, e.g., the animal models disclosed herein or known to those skilled in the art. In addition, in vitro assays may optionally be employed to help identify optimal dosage ranges.

In some aspects, the prophylactic and/or therapeutic regimens comprise titrating the dosages administered to the patient so as to achieve a specified measure of therapeutic efficacy. Such measures include a reduction in the cancer cell population in the patient.

In certain cases, the dosage of the agent of the invention in the prophylactic and/or therapeutic regimen is adjusted so as to achieve a reduction in the number or amount of cancer cells found in a test specimen extracted from a patient after undergoing the prophylactic and/or therapeutic regimen, as compared with a reference sample. Here, the reference sample is a specimen extracted from the patient undergoing therapy, wherein the specimen is extracted from the patient at an earlier time point. In one aspect, the reference sample is a specimen extracted from the same patient, prior to receiving the prophylactic and/or therapeutic regimen. For example, the number or amount of cancer cells in the test specimen is at least 2%, 5%, 10%, 15%, 20%, 30%, 40%, 50%, 60%, 70%, 80%, 90%, 95% or 99% lower than in the reference sample.

In some cases, the dosage of the agent of the invention in the prophylactic and/or therapeutic regimen is adjusted so as to achieve a number or amount of cancer cells that falls within a predetermined reference range. In these embodiments, the number or amount of cancer cells in a test specimen is compared with a predetermined reference range.

In other embodiments, the dosage of the agent of the invention in prophylactic and/or therapeutic regimen is adjusted so as to achieve a reduction in the number or amount of cancer cells found in a test specimen extracted from a patient after undergoing the prophylactic and/or therapeutic regimen, as compared with a reference sample, wherein the reference sample is a specimen is extracted from a healthy, noncancer-afflicted patient. For example, the number or amount of cancer cells in the test specimen is at least within 60%, 50%, 40%, 30%, 20%, 15%, 10%, 5%, or 2% of the number or amount of cancer cells in the reference sample.

In treating certain human patients having solid tumors, extracting multiple tissue specimens from a suspected tumor site may prove impracticable. In these cases, the dosage of the agent of the invention in the prophylactic and/or therapeutic regimen for a human patient is extrapolated from doses in animal models that are effective to reduce the cancer population in those animal models. In the animal models, the prophylactic and/or therapeutic regimens are adjusted so as to achieve a reduction in the number or amount of cancer cells found in a test specimen extracted from an animal after undergoing the prophylactic and/or therapeutic regimen, as compared with a reference sample. The reference sample can be a specimen extracted from the same animal, prior to receiving the prophylactic and/or therapeutic regimen. In specific embodiments, the number or amount of cancer cells in the test specimen is at least 2%, 5%, 10%, 15%, 20%, 30%, 40%, 50% or 60% lower than in the reference sample. The doses effective in reducing the number or amount of cancer cells in the animals can be normalized to body surface area (e.g., mg/m²) to provide an equivalent human dose.

The prophylactic and/or therapeutic regimens disclosed herein comprise administration of an agent of the invention or pharmaceutical compositions thereof to the patient in a single dose or in multiple doses (e.g., 1, 2, 3, 4, 5, 6, 7, 8, 10, 15, 20, or more doses).

In one aspect, the prophylactic and/or therapeutic regimens comprise administration of the agent of the invention or pharmaceutical compositions thereof in multiple doses. When administered in multiple doses, the agent or pharmaceutical compositions are administered with a frequency and in an amount sufficient to treat the condition. For example, the frequency of administration ranges from once a day up to about once every eight weeks. In another example, the frequency of administration ranges from about once a week up to about once every six weeks. In another example, the frequency of administration ranges from about once every three weeks up to about once every four weeks.

Generally, the dosage of an agent of the invention administered to a subject to treat cancer is in the range of 0.01 to 500 mg/Kg, e.g., in the range of 0.1 mg/Kg to 100 mg/Kg, of the subject's body weight. For example, the dosage administered to a subject is in the range of 0.1 mg/Kg to 50 mg/Kg, or 1 mg/Kg to 50 mg/Kg, of the subject's body weight, more preferably in the range of 0.1 mg/Kg to 25 mg/Kg, or 1 mg/Kg to 25 mg/Kg, of the patient's body weight. In another example, the dosage of an agent of the invention administered to a subject to treat cancer in a patient is 500 mg/Kg or less, preferably 250 mg/Kg or less, 100 mg/Kg or less, 95 mg/Kg or less, 90 mg/Kg or less, 85 mg/Kg or less, 80 mg/Kg or less, 75 mg/Kg or less, 70 mg/Kg or less, 65 mg/Kg or less, 60 mg/Kg or less, 55 mg/Kg or less, 50 mg/Kg or less, 45 mg/Kg or less, 40 mg/Kg or less, 35 mg/Kg or less, 30 mg/Kg or less, 25 mg/Kg or less, 20 mg/Kg or less, 15 mg/Kg or less, 10 mg/Kg or less, 5 mg/Kg or less, 2.5 mg/Kg or less, 2 mg/Kg or less, 1.5 mg/Kg or less, or 1 mg/Kg or less of a patient's body weight.

In another example, the dosage of an agent of the invention administered to a subject to treat cancer in a patient is a unit dose of 0.1 to 50 mg, 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In another example, the dosage of an agent of the invention administered to a subject to treat cancer in a patient is in the range of 0.01 to 10 g/m², and more typically, in the range of 0.1 g/m² to 7.5 g/m², of the subject's body weight. For example, the dosage administered to a subject is in the range of 0.5 g/m² to 5 g/m², or 1 g/m² to 5 g/m² of the subject's body's surface area.

In another example, the prophylactic and/or therapeutic regimen comprises administering to a patient one or more doses of an effective amount of an agent of the invention, wherein the dose of an effective amount achieves a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the agent of the invention.

In another example, the prophylactic and/or therapeutic regimen comprises administering to a patient a plurality of doses of an effective amount of an agent of the invention, wherein the plurality of doses maintains a plasma level of at least 0.1 μg/mL, at least 0.5 μg/mL, at least 1μg/mL, at least 2μg/mL, at least 5μg/mL, at least 6μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the agent of the invention for at least 1 day, 1 month, 2 months, 3 months, 4 months, 5 months, 6 months, 7 months, 8 months, 9 months, 10 months, 11 months, 12 months, 15 months, 18 months, 24 months or 36 months.

### Combination Therapy

In one example, the agents are administered in combination therapy, i.e., combined with other agents, e.g., therapeutic agents, that are useful for treating pathological conditions or disorders, such as various forms of cancer. The term “in combination” in this context means that the agents are given substantially contemporaneously, either simultaneously or sequentially. If given sequentially, at the onset of administration of the second compound, the first of the two compounds are in some cases still detectable at effective concentrations at the site of treatment.

The administration of a compound or a combination of compounds for the treatment of a neoplasia may be by any suitable means that results in a concentration of the therapeutic that, combined with other components, is effective in ameliorating, reducing, or stabilizing a neoplasia. The agent may be contained in any appropriate amount in any suitable carrier substance and is generally present in an amount of 1-95% by weight of the total weight of the composition. The agent may be provided in a dosage form that is suitable for parenteral (e.g., subcutaneously, intravenously, intramuscularly, or intraperitoneally) administration route. The agent may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Accordingly, in some examples, the prophylactic and/or therapeutic regimen comprises administration of an agent of the invention in combination with one or more additional anticancer therapeutics. In one example, the dosages of the one or more additional anticancer therapeutics used in the combination therapy is lower than those which have been or are currently being used to treat cancer. The recommended dosages of the one or more additional anticancer therapeutics currently used for the treatment of cancer can be obtained from any reference in the art including, but not limited to, Hardman et al. eds., Goodman & Gilman's The Pharmacological Basis of Basis of Therapeutics, 10th ed., McGraw-Hill, New York, 2001; Physician's Desk Reference (60.sup.th ed., 2006).

In some embodiments, the agent of the invention may be used in combination with one or more additional anticancer therapeutics. Examples of anticancer therapeutics include cis-retinoic acid, cyclophosphamide (Cytoxan®, Neosar®, Endoxan®), cisplatin (Platinol®), carboplatin (Paraplatin®), vincristine (Oncovin®, Vincasar PFS®, VCR), doxorubicin (Adriamycin ®, Rubex®), etoposide (Toposar®, VePesid®, Etopophos®,VP-16) , topotecan (Hycamtin®), busulfan (Myleran®, Busulfex®) and melphalan (Alkeran®, L-PAM, Evomela®), or thiotepa (Thioplex®, Tepadina®).

In some embodiments, the anticancer therapeutics may be co-administered with one or more steroids, including methylprednisolone (Depo-Medrol®, Solu-Medrol®, Medrol®), prednisone (Sterapred®, Prednisone Intensol), dexamethasone (Decadron®), hydrocortisone (Cortef®), or Adrenocorticotropic hormone derivatives, including tetracosactide (synacthen®, tetracosactrin®, cosyntropin®).

In some embodiments, the prophylactic and/or therapeutic regimen comprises administration of an agent of the invention in combination with a combination chemotherapy agent. In some embodiments, the combination chemotherapy agent includes busulfan (Myleran®, Busulfex®), carboplatin (Paraplatin®) or cisplatin (Platinol®), cyclophosphamide (Cytoxan®, Neosar®, Endoxan®), doxorubicin (Adriamycin®, Rubex®), etoposide (Toposar®, VePesid®, Etopophos®, VP-16), irinotecan (Onivyde®), temozolomide (Temodal®, or ifosfamide (Ifex®), thiotepa (Tepadina®), melphalan (Evomela®), topotecan (Hycamtin®), or vincristine (Margibo®, Vincasar PFS®). In some embodiments, this treatment is followed by a stem cell transplant. The chemotherapy agents may be used in combination with other treatments in a monotherapy (i.e., a single chemotherapy agent) or as a polytherapy (i.e., more than one chemotherapy agent. Polytherapties may include any combination of agents. One common polytherapy includes isplatin (or carboplatin), cyclophosphamide, doxorubicin, vincristine, and etoposide.

In some cases, the prophylactic and/or therapeutic regimen comprises administration of an agent of the invention in combination with an immunosuppressant agent such dinutuximab (Unituxin®) with or without cis-retinoic acid, or rituximab (Rituxan®).

The agent of the invention and the one or more additional anticancer therapeutics can be administered separately, simultaneously, or sequentially. In various aspects, the agent of the invention and the additional anticancer therapeutic are administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, 96 hours apart, 120 hours part, or 168 hours apart. In another example, two or more anticancer therapeutics are administered within the same patient visit.

In certain aspects, the agent of the invention and the additional anticancer therapeutic are cyclically administered. Cycling therapy involves the administration of one anticancer therapeutic for a period of time, followed by the administration of a second anticancer therapeutic for a period of time and repeating this sequential administration, i.e., the cycle, in order to reduce the development of resistance to one or both of the agents, to avoid or reduce the side effects of one or both of the agents, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to the agent, to avoid or reduce the side effects of one of the agent, and/or to improve the efficacy of the agent.

In another example, the agents are administered concurrently to a subject in separate compositions. The combination the agents of the invention may be administered to a subject by the same or different routes of administration.

When an agent of the invention and the additional anticancer therapeutic are administered to a subject concurrently, the term “concurrently” is not limited to the administration of the agent at exactly the same time, but rather, it is meant that they are administered to a subject in a sequence and within a time interval such that they can act together (e.g., synergistically to provide an increased benefit than if they were administered otherwise). For example, the agents may be administered at the same time or sequentially in any order at different points in time; however, if not administered at the same time, they should be administered sufficiently close in time so as to provide the desired therapeutic effect, preferably in a synergistic fashion. The combination of the agents can be administered separately, in any appropriate form and by any suitable route. When the components of the combination the agents are not administered in the same pharmaceutical composition, it is understood that they can be administered in any order to a subject in need thereof. For example, an agent of the invention can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the additional anticancer therapeutic, to a subject in need thereof. In various aspects, the agents are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one example, the agents are administered within the same office visit. In another example, the combination the agents of the invention are administered at 1 minute to 24 hours apart.

### Release of Pharmaceutical Compositions

Pharmaceutical compositions according to the invention may be formulated to release the agents substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neoplasia by using carriers or chemical derivatives to deliver the agent to a particular cell type (e.g., neoplastic cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the agent. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion, or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the agent that reduces or ameliorates a neoplasia, the composition may include suitable parenterally acceptable carriers and/or excipients. The agent may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active antineoplastic therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl, or n-propyl p-hydroxybenzoate). In cases where one of the compounds is only sparingly or slightly soluble in water, a dissolution enhancing or solubilizing agent can be added, or the solvent may include 10-60% w/w of propylene glycol.

### Controlled Release

Controlled release of parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Pharmaceutical Kits

The present compositions may be assembled into pharmaceutical kits for use in ameliorating a neoplasia. Pharmaceutical kits according to this aspect of the invention comprise a carrier means, such as a box, carton, tube, or the like, having in close confinement therein one or more container means, such as vials, tubes, ampoules, or bottles. The pharmaceutical kits of the invention may also comprise associated instructions for using the agent of the invention.

### Methods of use

In some aspects, the present invention is directed to methods of treating diseases or disorders involving aberrant (e.g., dysfunctional or dysregulated) EP300 activity, referred herein as “EP300-dependent” diseases or disorders, and treatment entails administration of a therapeutically effective amount of a selective degrader of EP300 (e.g., JQAD1) or a pharmaceutically acceptable salt or stereoisomer thereof, to a subject in need thereof

These EP300-dependent diseases or disorders are characterized by aberrant EP300 activity (e.g., elevated levels of EP300 or otherwise functionally abnormal EP300 relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health. In some embodiments, compounds of the application may be useful in the treatment of cell proliferative diseases and disorders (e.g., cancer or benign neoplasms). As used herein, the term “cell proliferative disease or disorder” refers to the conditions characterized by deregulated or abnormal cell growth, or both, including noncancerous conditions such as neoplasms, precancerous conditions, benign tumors, and cancer.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

The term “sample” as used herein refers to a biological sample obtained for the purpose of evaluation in vitro. Exemplary tissue samples for the methods described herein include tissue samples from NB tumors or the surrounding tumor microenvironment (i.e., the stroma). The tumor microenvironment is typically comprised of proliferating tumor cells, the tumor stroma, blood vessels, infiltrating inflammatory cells and a variety of associated tissue cells. The tumor microenvironment is unique and emerges over the course of tumor progression as a result of its interactions with the host. It is created by and dominated by the tumor, which effects and drives molecular and cellular events taking place in surrounding tissues. With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma or serum). Preferred samples are whole blood, serum, plasma, or urine. A sample can also be a partially purified fraction of a tissue or bodily fluid.

A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

Exemplary types of non-cancerous (e.g., cell proliferative) diseases or disorders that may be amenable to treatment with the selective degraders of EP300 of the present invention include inflammatory diseases and conditions, autoimmune diseases, neurodegenerative diseases, heart diseases, viral diseases, chronic and acute kidney diseases or injuries, metabolic diseases, and allergic and genetic diseases.

In some embodiments, the methods are directed to treating subjects having cancer. Broadly, the compounds of the present invention may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma, and multiple myeloma. Adult tumors/cancers and pediatric tumors/cancers are included. The cancers may be vascularized, or not yet substantially vascularized, or non-vascularized tumors.

In some embodiments, the selective degraders of EP300 of the present invention are used to treat a caner with dysregulated or dysfunctional EP300 (i.e., EP300-dependent cancers), for example, NB, rhabdomyosarcoma, stomach cancer, brain cancer, pancreatic cancer, colorectal cancer (Gayther et al., Nat Genet 24:300-3 (2000)), breast cancer (Sobczak et al., Cancers (Basel) 11:1539 (2019)), lung cancer, lung squamous cell carcinoma, squamous cell carcinoma, prostate cancer, ovarian cancer, esophageal cancer, pancreatic cancer, retinoblastoma, cervical cancer, endometrial cancer, medulloblastoma, diffuse large B-Cell lymphoma, acute lymphoblastic leukemia, bladder urothelial carcinoma, monocytic leukemia, head and neck squamous cell carcinoma ((SCCHN)), hematologic cancers, Adult T-cell leukemia lymphoma (ATLL), or NUT midline carcinoma.

Furthermore, EP300 has been described as a driver gene in bladder urothelial carcinoma where EP300 inhibition may benefit in addition to anti-PD-1 or anti-PD-L1 immunotherapy (Meng et al., Mol. Ther. Oncolytics 20:410-421 (2021); Chang et al., Exp. Mol. Med. 51:1-17 (2019)). In monocytic leukemia, MLL-EP300 oncoproteins have been described, see, Ohnishi et al., Eur. J. Haematol. 81:475-80 (2008). In SCCHN, high CD8+T-cell inflamed phenotypes are enriched in EP300 mutations (Saloura et al., Oral Oncol. 96:77-88 (2019)). In ATLL, 20% of cases with mutations in epigenetic and histone modifying genes had a mutation in EP300 (Shah et al., Blood 132:1507-1518 (2018)). In NUT midline carcinoma, EP300 is implicated in feed-forward regulatory loops leading to propagation of the oncogenic chromatin complex in bromodomain-containing protein 4 (BRD4)-NUT oncoprotein-induced cancer cells (Alekseyenko et al., Proc. Natl. Acad. Sci. U.S.A. 114:E4184-E4192 (2017)).

The practice of the present invention employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Materials and Methods

The following materials and methods were utilized to generate the results described herein. Chemical probes and biology reagents generated in this study are available for research purposes through material transfer agreement (MTA) or through the commercial vendors. Data availability and experimental models and subject matter details. RNA-seq and ChIP-seq data have been deposited in the Gene Expression Omnibus (GEO) database under SuperSeries accession number GSE159617, which is comprised of SubSeries accession numbers GSE159613, GSE159614, GSE159615 and GSE159616. Code used in this study is described in the experimental details and is available upon request.

Cell lines. 293T, Kelly, BE2C, NGP, NB69 and SIMA neuroblastoma cell lines were obtained from the American Type Culture Collection (BE2C, 293T), European Collection of Authenticated Cell Cultures (NB69), and the German Collection of Microorganisms and Cell Cultures GmbH (DSMZ) (Kelly, NGP, SIMA). S2 cells were a gift of Dr. Karen Adelman (Harvard Medical School, Boston, MA). Cell lines used for the exome-scale CRISPR—Cas9 screen and PRISM analyses have been previously described in Corsello et al. Nat. Cancer 1:235-248 (2020) and Meyers et al. Nat. Genet. 49:1779-1784 (2017). All cell lines were short tandem repeat (STR) tested for identity. Neuroblastoma cell lines were cultured in Roswell Park Memorial Institute (RPMI) media containing 10% heat-inactivated fetal bovine serum and 1% penicillin-streptomycin and validated to be free of Mycoplasma species by routine testing.

Chemicals. Compounds C646 and CBP30 were obtained from Tocris TM Biosciences. Bortezomib, MLN4924, and thalidomide were obtained from Sigma-Aldrich®, and pomalidomide and lenalidomide were obtained from Target Molecule Corp. All other chemicals were synthesized and characterized in Qi Lab. Compounds JQAD1 and Biotin-JQAD1 were designed and synthesized based on the scheme listed in the below examples. The structure and purity of these compounds were further confirmed by nuclear magnetic resonance (NMR) and liquid chromatography—mass spectrometry (LC-MS). Animals. 8-week-old female NOD. Cg-Prkdc^{scid}Il2rg^{tm1Wjl}/SzJ (NSG™) mice (Jackson Laboratories, catalog #: 0005557) were used for tumor xenograft studies. For maximally tolerated dose testing, C57BL/6-Crbdm^{tm1.1Ble}/J mice (Jackson Laboratories, catalog #: 032487) were used. For pharmacokinetic studies, Crl:CD1(ICR) mice (Charles River Laboratories, catalog #022) were used. Additional details, including reagent or resource name, source, and identifier, are listed in Table 2.

Quantification and statistical analysis. Data from the chromatin immunoprecipitation coupled to high-throughput sequencing (ChIP-Seq) and CRISPR—Cas9 screens were analyzed as described. Animal experiments were analyzed by mixed-effects modeling and two-sided analysis of variance (ANOVA) for tumor volume and weight means, and by the log- rank test for survival. Other data were analyzed with one- or two-sided ANOVA with post hoc Tukey tests, two-sided t-tests, or one- or two-sided Fisher exact tests as appropriate for multiple or pair wise comparisons. Statistical significance was defined as ap<0.05 unless otherwise stated. Data were analyzed with GraphPad Prism 7.01, and all error bars represent standard deviation unless otherwise noted.

TABLE 2
Key resources

| | | |
|---|---|---|
| REAGENT or RESOURCE | SOURCE | IDENTIFIER |

Antibodies

| | | |
|---|---|---|
| Rabbit polyclonal anti-EP300 | Abcam | Cat#Ab10485 |
| Rabbit monoclonal anti-CBP (clone D6C5) | Cell Signaling | Cat #7389 |
| | Technology | |
| Rabbit polyclonal anti-PARP1 | Cell Signaling | Cat #9542 |
| | Technology | |
| Rabbit polyclonal anti-Cleaved Caspase 3 (Asp175) | Cell Signaling | Cat #9661 |
| | Technology | |
| Rabbit polyclonal anti-β-Actin | Cell Signaling | Cat #4967 |
| | Technology | |
| Rabbit polyclonal anti-H3K27ac | Abcam | Cat#Ab4729 |
| Mouse monoclonal anti-TFAP2β (clone C-6) | Santa Cruz | Cat#sc-390119 |
| | Biotechnology | |
| Rabbit polyclonal anti-TFAP2β | Cell Signaling | Cat #2509 |
| | Technology | |
| Rabbit monoclonal anti-MYCN (clone D1V2A) | Cell Signaling | Cat #84406 |
| | Technology | |
| Mouse monoclonal anti-Total H3 (clone 1B1B2) | Cell Signaling | Cat #14269 |
| | Technology | |
| Rabbit monoclonal anti-CRBN (clone D8H3S) | Cell Signaling | Cat #71810 |
| | Technology | |
| Mouse monoclonal anti-Vinculin (clone V284) | EMD Millipore/ | Cat#05-386 |
| | Sigma Aldrich | |
| Rabbit monoclonal anti-GATA3 (clone D13C9) | Cell Signaling | Cat #5852 |
| | Technology | |
| Mouse monoclonal anti-HAND2 (clone A-12) | Santa Cruz | Cat #sc-398167 |
| | Biotechnology | |
| Rabbit polyclonal normal IgG | Santa Cruz | Cat #sc-3888 |
| | Biotechnology | |
| Mouse monoclonal anti-CAS9 (clone 7A9-3A3) | Cell Signaling | Cat #14697 |
| | Technology | |
| Mouse monoclonal anti-ASCL1 (clone D-7) | Santa Cruz | Cat#/sc-374104 |
| | Blotechnology | |

Bacterial and Virus Strains

| | | |
|---|---|---|
| One Shot Stbl3 chemically competent | Thermo-Fisher | Cat#C737303 |
| E. coli cells | Scientific | |

Biological Samples
Chemicals, Peptides, and Recombinant Proteins

| | | |
|---|---|---|
| (R,S)-JQAD1 | This study | N/A |
| (S,S)-JQAD1 | This study | N/A |
| A485 | Synthesized in Qi lab | As detailed in |
| | | (Michaelides, 2018 |
| | | #512) |
| C646 | Tocris Biosciences | Cat# 4200/10 |
| SGC-CBP30 | Tocris Biosciences | Cat# 4889/10 |
| Thalidomide | Sigma-Aldrich | Cat# T144 |
| | Biosciences | |
| Lenalidomide | Target Molecule Corp | Cat# T1642 |
| Pomalidomide | Target Molecule Corp | Cat# T2384 |
| Bortezomib | Sigma-Aldrich | Cat#5043140001 |
| | Biosciences | |
| MLN4924 | Sigma-Aldrich | Cat#5.05477 |
| | Biosciences | |
| Lipofectamine 2000 | Thermo-Fisher | Cat# 11668019 |
| | Scientific | |
| Polybrene reagent | Sigma-Aldrich | Cat# TR-1003 |
| Blasticidin HCl | Thermo-Fisher | Cat# A1113903 |
| | Scientific | |
| High capacity streptavidin agarose rasin | Pierce Biotechnology | Cat# 20361 |
| Sequencing-grade trypsin | Promega | Cat# V5111 |
| 10% hydroxypropyl β-cyclodextrin | Sigma-Aldrich | Cat#H107-5G |

Critical Commercial Assays

| | | |
|---|---|---|
| Mycoalert mycoplasma testing kit | Lonza | Cat# LT07-118 |
| NE-PER nuclear lysate kit | Thermo-Fisher | Cat# 78833 |
| | Scientific | |
| Total histone extraction kit | Epigentek | Cat# OP-0006-100 |
| Cell Titer-glo | Promega | Cat# G7570 |
| AlphaLISA Assay | Perkin Elmer | Cat# 6760000K |
| Leica Biosystems Refine Detection Kit | Leica Biosystems | Cat# DS9800 |
| ¹³C₆ ¹⁵N₂ L-lysine and ¹³C₆ ¹⁵N₄ L-arginine | Thermo-Fisher | Cat#89982 |
| SILAC RPMI labelling kit | Scientific | |
| Dynabeads M270 magnetic beads co- | Thermo-Fisher | Cat#14321D |
| immunoprecipitation kit | Scientific | |
| ERCC RNA control spike in mix | Thermo-Fisher | Cat#4456740 |
| | Scientific | |

Deposited Data

| | | |
|---|---|---|
| Raw and analyzed data | This paper | GEO: GSE159617 |
| GSE159613-ChIP-seq | This paper | GEO: GSE159617 |
| GSE159614-ChIP-Rx | This paper | GEO: GSE159617 |
| GSE159615-RNAseq | This paper | GEO: GSE159617 |
| GSE159616-RNAseq | This paper | GEO: GSE159617 |
| GSE120074-ChIP-seq | Wang et al. 2019 | ASCL1 ChIP-seq in |
| | | Kelly cells |
| GSE94822-ChIP-seq, ATAC-seq | Durbin AD et al. 2018 | Core-regulatory |
| | | circuitry ChIP-seq. |
| | | ATAC-seq In NB cell |
| | | lines |
| GSE65664-ChIP-seq | Oldridge DA et al. | ChIP-seq to |
| | 2015. | H3K27ac |

Experimental Models: Cell Lines

| | | |
|---|---|---|
| Human: 293T cells | ATCC | Cat# CRL-3216 |
| Human: Kelly neuroblastoma cells | DSMZ | Cat# ACC-355 |
| Human: BE2C neuroblastoma cells | ATCC | Cat# CRL-2268 |
| Human: SIMA neuroblastoma cells | DSMZ | Cat# ACC-164 |
| Human: NGP neuroblastoma cells | DSMZ | Cat# ACC-676 |
| Human: NB69 neuroblastoma cells | ECACC | Cat# 99072802 |
| Human: Other human cancer cell lines used in | Courtesy of the Broad | N/A |
| screening | Institute | |
| Drosophila: S2 cells | Laboratory of Dr. | N/A |
| | Karen Adelman | |

Experimental Models: Organisms/Strains

| | | |
|---|---|---|
| C57BL/6-Crbn^{tm1.1Ble}/J | Jackson Laboratory | Stock No: 032487 |
| Crl:CD1(ICR) | Charles River | Strain Code: 022 |
| | Laboratories | |
| NOD.Cg-Prkdc^{scid} Il2rg^{tm1Wj1}/SzJ | Jackson Laboratories | Stock No: 005557 |

Oligonucleotides

| | | |
|---|---|---|
| sgRNA sequences used to for CRISPR-cas9- | This study | N/A |
| mediated gene knockout, see Table S3 | | |

Recombinant DNA

| | | |
|---|---|---|
| Lenticas9-Blast | Sanjana et al., 2014 | Plasmid#52962 |
| pLKO.5-EGFP | Heckl et al., 2014 | Plasmid#57822 |
| pMD2.G | Laboratory of Didier | Addgene |
| | Trono | Plasmid#12259 |
| psPAX2 | Laboratory of Didier | Addgene |
| | Trono | Plasmid#12260 |

Software and Algorithms

| | | |
|---|---|---|
| Bedtools | Quinlan AR and Hall | https://github.com/ |
| | IM, 2010. | arq5x/bedtools2 |
| GENRE | Mariani L et al. 2017. | http://thebrain.bwh. |
| | | harvard.edu/glossary- |
| | | GENRE/download.html |
| matchPWM within Biostrings | Mariani L et al. 2017. | https://github.com/ |
| | | Bioconductor/Blostrings/ |
| | | blob/master/R/matchPWM.R |
| Bowtie 1.2.2 | Langmead and | http://bowtie- |
| | Salzberg, 2012 | bio.sourceforge.net/ |
| | | index.shtml |
| MACS 1.4.2 | Feng J et al., 2012. | https://github.com/macs3- |
| | | project/MACS |
| Samtools | Li et al., 2009 | http://samtools.github.io |
| bamtoGFF | Lin CY et al., 2016 | https://github.com/ |
| | | BradnerLab/pipeline |
| ROSE | Lin CY et al., 2016 | https://github.com/ |
| | | BradnerLab/pipeline |
| FlowJo v.10.7 | BD Biosciences | |
| IGV 2.4.10 | Broad Institute | |

Other

| | | |
|---|---|---|
| CCLE Proteomics Data | Broad Institute | N/A |
| CCLE mRNA Expression Data | Broad Institute | N/A |
| DepMap Dependency Data | Broad Institute | N/A |
| R2 Database Kaplan-Meier/Expression Data | R2 Database | N/A |

### Example 2: Cell Viability Assays

Cells were infected with lentiviruses encoding sgRNAs or treated with compounds as described. Colony assays were performed by replating cells at 500 cells per well in 6-well dishes and grown in regular growth media for 10 d before 100% methanol fixation, 0.05% crystal violet staining, and quantitation. Experiments were completed in triplicate; data shown are the average of three independent experiments. Cell Titer-Glo® assay was performed as per the manufacturer's instructions (Promega®). Briefly, 1000 cells/well were plated into 96-well plates and treated with a range of compound dosing. Cell viability was measured at the noted time points based on luminescence by the Cell Titer-Glo® assay (Promega®) and read on an Envision 2104 (PerkinElmer®, USA) according to the manufacturer's protocol.

### Example 3: Propidium Iodide-Cell Cycle Analysis

Cells were infected with lentiviruses encoding single guide RNAs (sgRNAs) or treated with compounds as described for the noted length of time. Cells were then liberated from adhesion to the plate using a sterile spatula (Coming®) followed by centrifugation, aspiration of media, and resuspension in hypotonic citrate-propidium iodide (PI) solution for 30 minutes at 37° C. (Tate et al. Cytometry 4:211-215 (1983)). Nuclei were stabilized using 5M NaCl prior to analysis on a FACSAria™ II (BD Biosciences). Analysis of cell cycle phases was performed using FlowJo® v10.7 (BD Biosciences).

### Example 4: Lentiviral Infection

Stable and inducible cas9-expressing cell lines were generated using lentiviral particles produced in 293T cells. Briefly, lenticas9 (plasmid #52962), pCW-cas9-Blast (#83481), and pLKO.5-EGFP (#57822) plasmids were obtained from Addgene. Plasmids were transfected using lipofectamine 2000 (Invitrogen™) along with pMD2.G (Addgene Plasmid #12259) and psPAX2 (#12260) to generate viral particles by standard methodologies.

sgRNAs targeting individual genes were subcloned by standard methodologies within pLKO.5-EFGP. Kelly, SIMA, BE2C, and NGP cells were infected with lenticas9 followed by blasticidin selection. Stable expression of cas9 was established by western blotting of protein lysates using cas9 antibody (Cell Signaling Technology®). Following infection of pLKO.5-EGFP-sgRNA lentivirus, cells were cultured for the identified times prior to evaluation. BE2C cells were infected with pLC-zsgreen or pLC-CRBN lentiviruses and selected using 500 μg/mL hygromycin (Invitrogen™)

### Example 5: Western Blotting, Immunoprecipitation and Proteomic Analysis

Cells growing in culture were lysed for whole cell lysates as described in Durbin et al. Nat. Genet. 50:1240-1246 (2018) and Wang et al. Nat. Commun. 10:5622 (2019). Nuclear lysates were prepared using the NE-PER® nuclear lysate kit (Thermo Scientific™) according to the manufacturer's protocol. Chromatin lysates were prepared with the total histone extraction kit (Epigentek). Briefly, equivalent amounts of protein were resolved by western blotting using 4-12% Bis-Tris NuPAGE™ gels (Thermo-Fisher Scientific) prior to transfer, and immunoblotting using primary antibodies to: MYCN (1:1000, Cell Signaling Technology®), H3K27ac (1:1000, Abcam), total H3 (1:1000, Cell Signaling Technology®), EP300 (1:1000, Abcam), CBP (1:500, Cell Signaling Technology®), Cas9 (1:1000, Cell Signaling Technology®), cleaved-PARP1 (1:1000, Cell Signaling Technology), cleaved Caspase-3 (1:1000, Cell Signaling Technology®), β-actin (1:1000, Cell Signaling Technology®), GATA3 (1:1000, EMD Millipore™), TFAP2β (1:1000, Cell Signaling Technology®), Vinculin (1:1000, EMD Millipore™), CRBN (1:1000, Cell Signaling Technology®), HAND2 (1:1000, Santa Cruz Biotechnology). Secondary antibodies were horseradish peroxidase (HRP)-conjugated anti-rabbit or anti-mouse (1:5000, Santa Cruz Biotechnology), incubated prior to exposure to enhanced chemiluminescence reagents (GE, Amersham). For immunoprecipitation, equal amounts of protein were diluted in buffer C as described in Mansour et al. Science 346:1373-1377 (2014) and incubated with antibodies covalently conjugated to Dynabeads™ M-270 beads (Thermo-Fisher Scientific) overnight according to the manufacturer's directions. Antibodies used included: H3K27ac, EP300 (Abcam), CBP, TFAP2β (Cell Signaling Technology®), rabbit immunoglobulin G (IgG) (Santa Cruz Biotechnology®). Immunoprecipitated protein was isolated as per the manufacturer's directions and subjected to western blotting, as described above, or mass spectrometry.

### Example 6: SILAC and H3K27ac co-IP Mass Spectrometry

For analysis of JQAD1 effects on the nuclear proteome, Kelly cells were labelled with both heavy ¹³C₆ ¹⁵N₂ L-lysine and ¹³C₆ ¹⁵N₄ L-arginine (“heavy” labelled cells) or normal L-lysine and L-arginine (“light” labelled cells). Heavy-labelled cells were treated with 1 μM JQAD1, and light-labelled cells were treated with equivalent concentrations of DMSO for 24 h, prior to preparation of nuclear lysates using the NE-PER® nuclear lysis kit (Thermo Fisher Scientific). Untreated heavy and light cells were also lysed for nuclear protein as a control. 750 μg of heavy and light nuclear lysate was pooled and subjected to trichloroacetic acid precipitation by standard methodologies. Precipitated protein was resuspended in 4× Laemmli sample buffer, boiled and separated by SDS-PAGE by standard methodologies. Gels were divided into two sections based on molecular weight, cut into 1 mm³ pieces and subjected to a modified in-gel trypsin digestion procedure (Shevchenko et al. Anal. Chem. 68:850-858 (1996)). Briefly, gel pieces were washed, dehydrated with acetonitrile, and rehydrated in 50 mM ammonium bicarbonate solution containing 12.5 ng/μl modified sequencing-grade trypsin (Promega®) at 4° C. Samples were then washed and incubated in 50 mM ammonium bicarbonate solution at 37° C. for >16 h. Peptides were extracted by washing in 50% acetonitrile and 1% formic acid and dried by speed-vac. For analysis, samples were reconstituted in high-performance liquid chromatography (HPLC) solvent A (2.5% acetonitrile, 0.1% formic acid) and loaded onto a nano-scale reverse-phase HPLC capillary column (2.6 μm C18 spherical silica beads in a fused silica capillary) as described in Peng and Gygi, J. Mass. Spectrom. 36:1083-91 (2001). Samples were loaded via a FAMOS™ autosampler (LC Packings, San Francisco, CA). Peptides were eluted with increasing concentrations of solvent B (97.5% acetonitrile, 0.1% formic acid), and subjected to electrospray ionization and then entered into an LTQ Orbitrap Velos Pro™ ion-trap mass spectrometer (Thermo Fisher Scientific). Peptides were detected, isolated, and fragmented to produce a tandem mass spectrum of specific fragment ions for each peptide. Peptide sequences and protein identity were determined by matching protein databases with the acquired fragmentation pattern by Sequest® (Thermo Fisher Scientific) (Eng et al. J. Am. Soc. Mass Spectrom. 5:976-89 (1994)). All databases include a reversed version of all peptide sequences, and the data were filtered to between a one and two percent peptide false discovery rate. Treatments were repeated three independent times and subjected to mass spectrometry three independent times. Sum ratios of peptides and assigned proteins were used to calculate changes in abundance, comparing heavy to light peptides at 24 h (treated) samples, normalized against 0 h controls. Across three independent mass spectrometry assessments, 2493 proteins were detected, filtered for proteins present at detectable rates at 0 h. Protein abundance was determined by student's t-test, comparing 0 h abundance to 24 h abundance.

For co-immunoprecipitation/mass spectrometry analysis of H3K27ac, BE2C and Kelly cells growing in regular growth media were treated to collect nuclear lysates as described above. 750 μg of nuclear protein was immunoprecipitated using Dynabeads™ M270 magnetic beads covalently bound with H3K27ac antibody (Abcam) or normal rabbit IgG (Santa Cruz Biotechnology®) as detailed for >16 h at 4° C. prior to washing and elution of immunoprecipitated protein as per the manufacturer's instructions (Invitrogen™). Eluted protein was subjected to trichloroacetic acid precipitation, trypsin digestion and mass spectrometry as described above. Two independent co-immunoprecipitation/mass spectrometry experiments were performed in each of BE2C and Kelly cells. In total, 366 and 281 proteins were identified to interact with H3K27ac and rabbit IgG in Kelly cells, and 1323 and 1113 proteins identified in BE2C cells. Proteins identified by both H3K27ac and rabbit IgG were removed as non-specific binders, resulting in 167 and 492 protein interactors with H3K27ac in Kelly and BE2C cells, respectively. High confidence proteins were defined as the subset found in both Kelly and BE2C cells. This subset was a total of n=35 proteins, demonstrated in Table 3, with gene identities and function being identified using Gene Ontology and PANTHER analyses (The Gene Ontology Consortium, Nucleic Acids Res 43:D1049-56 (2015); Mi et al. Nat. Protoc. 8:1551-1566 (2013)).

TABLE 3
H3K27ac-associated proteins identified by
co-immunoprecipitation mass spectrometry analysis.

| Protein | PANTHER | “Other” |
|---|---|---|
| Interactor | Protein Class | PANTHER Class |
| EP300 | Histone modifying | |
| | enzyme | |
| CBP | Histone modifying | |
| KAT7 | Histone modifying | |
| TFAP2E | Transcription Factor | |
| GATA3 | Transcription Factor | |
| DLX6 | Transcription Factor | |
| TF AM | Transcription Factor | |
| SUPT16H | Chromatin Binding | |
| SCML2 | Chromatin Binding | |
| MEAF6 | Chromatin Binding | |
| VPS72 | Chromatin Binding | |
| EP400 | Chromatin Binding | |
| CENPV | Chromatin Binding | |
| CHAF1A | Chromatin Binding | |
| BCL7C | Chromatin Binding | |
| ASF1A | Chromatin Binding | |
| MRGBP | Chromatin Binding | |
| RNF2 | Chromatin Binding | |
| TFIP11 | RNA Splicing Factor | |
| BCAS2 | RNA Splicing Factor | |
| SRSF10 | RNA Splicing Factor | |
| MAGOH | RNA Splicing Factor | |
| LSM2 | RNA Splicing Factor | |
| SF3A2 | RNA Splicing Factor | |
| PPIH | RNA Splicing Factor | |
| LENG8 | Scaffold/Adaptor Protein | |
| PQBP1 | Scaffold/Adaptor Protein | |
| SDF2L1 | Chaperone | |
| NASP | Chaperone | |
| RPS17 | Other | Ribosomal Protein |
| NCBP2 | Other | RNA Binding Protein |
| CALM2 | Other | Calmodulin-related |
| MFAP1 | Other | Extracellular Matrix |
| KIF18B | Other | Microtubule Binding |
| RAD23B | Other | DNA Binding Protein |

Table 3 shows proteins identified to interact with H3K27ac in both BE2C and Kelly cells, resolved by co-immunoprecipitation/mass spectrometry. Normal rabbit IgG was used as a negative control. These high-confidence proteins were identified in two independent co-IP/mass spectrometry reactions per cell line, found in both Kelly and BE2C cells and not in IgG controls. Also demonstrated is the protein annotation through evolutionary relationship (PANTHER) protein class for each protein.

### Example 7: In Vivo Studies

Protocols approved by the Dana—Farber Cancer Institute Animal Care and Use Committee were followed. Animals were maintained according to institutional guidelines. 8-week-old female NOD .Cg-Prkdc^{scid}Il2rg^{tm1Wjl}/SzJ (NSG™) mice (Jackson Laboratories, catalog #: 0005557) were used for tumor xenograft studies. For maximally tolerated dose testing, C57BLI6-Crbn^{tm1.lBle}/J mice (Jackson Laboratories, catalog #: 032487) were used. For pharmacokinetic studies, Crl:CD1(ICR) mice (Charles River Laboratories, catalog #022) were used.

For toxicity studies, four female CD1 mice (Charles River Laboratories) were injected intraperitoneally (IP) with single doses of 10 mg/Kg (R,S)-JQAD1 solubilized in 10% hydroxypropyl β-cyclodextrin (Sigma-Aldrich®) in sterile water. Following injection, blood concentration of (R,S)-JQAD1 was measured by serial measurements of animal serum at time points out to 24 h, by liquid chromatography with tandem mass spectrometry (LC-MS/MS) analysis. Pharmacokinetics were performed at ChemPartner in Shanghai, China, using LC-MS/MS method and pharmacokinetics parameters (Tₘₐₓ, Cₘₐₓ, T_{1/2}, AUC, etc.) calculated with WinNonlin® V 6.2 statistics software (Pharsight Corporation) using a noncompartmental model. For maximally tolerated dose (MTD) testing, six female CD1 mice were treated with daily IP doses of (R,S)-JQAD1 at 10, 20, or 40 mg/kg. Animals were monitored for animal weight, grooming and behavior daily without noted effects. For MTD testing in humanized CRBN knockin (Balb/c CRBN^{ILE391VAL}) (Jackson Laboratories), 6 mice per treatment group were treated with either vehicle control or (R,S)-JQAD1 at 40 mg/kg/day by IP injection. Animal weights, behavior and grooming were monitored daily, for a total of 21 days. At day 14, three mice per treatment group were sacrificed and tissues fixed for immunohistochemistry. Blood samples were obtained by retro-orbital puncture and blood analyzed at the Small Animal Imaging Facility at Beth Israel Deaconess Medical Center (Boston, MA), on a Hemavet® 9500FS (Drew Scientific) for blood counts, creatinine, AST, ALT, ALP, GGTP, and BUN measurements.

For tumor studies, eight-week-old female NSG™ mice (Jackson Laboratories) were subcutaneously implanted with 2.5×10⁶ Kelly cells in 50% matrigel/PBS. Mice were assigned to three groups: vehicle (n=11), JQAD1 (40 mg/kg/day) (n=12) or JQAD1 (40 mg/kg, twice daily) (n=12) by IP injection. Treatment with small-molecule inhibitors was initiated once tumors engrafted and reached 100-150 mm³. Mice were treated for 21 days and then followed for survival. Tumors were measured by calipers, and mice were weighed every three days. Animals were euthanized according to institutional guidelines when tumors reached 2,000 mm in length or width, or if animals became moribund. Tumor sizes were compared at each time point by two-way ANOVA with post-hoc Tukey tests. Tumor growth curve kinetics were analyzed by both logistic regression and mixed-effects two-way ANOVA with post-hoc Tukey tests to determine whether growth kinetics differed between treatment groups.

Separately, eight animals were xenografted as described above, and treated with vehicle (n=4) or JQAD1 (n=4) at 40 mg/kg IP daily for 14 days. These animals were sacrificed at day 14, following which tumor was extracted, and divided for immunohistochemical analysis or analysis of RNA expression by RNA sequencing (RNA-seq).

### Example 8: Immunohistochemistry

Immunohistochemistry was performed on the Leica® Bond™ III automated staining platform. Antibody EP300 from Cell Signaling Technology®, catalog number 86377, clone D8Z4E, was run at 1:50 dilution using the Leica® Biosystems Refine Detection Kit with citrate antigen retrieval. Antibody KAT3A/CBP (catalog number ab2832, polyclonal, Abcam) was run at 1:200 using the Leica® Biosystems Refine Detection Kit with ethylenediaminetetraacetic acid (EDTA) antigen retrieval.

### Example 9: RNA-seq and Analyses

For in vitro analyses, total RNA was extracted from control A485 or JQAD1 treated Kelly cells using TRIzol™ reagent (Ambion). Prior to extraction, exogenous spike-in of synthetic External RNA Control Consortium (ERCC) RNA controls were added based on cell number (Ambion). Samples were treated with RNAse-free DNAse I and spin purified using the Qiagen® RNeasy Kit (Qiagen®). Purified RNA samples were subjected to library construction with poly-adenylation preparation and sequencing using the Illumina NextSeq® 500 (paired end, 75bp reads).

RNA-seq reads were aligned to a reference index containing the sequences of the hg19 revision of the human reference genome and the ERCC spike-in probes using hisat 2.1.0. Expression was quantified using sorted BAMs, a gene reference built using ERCC sizes and RefSeq genes downloaded Jul. 15, 2017, and htseq-count with parameters -I gene_id- stranded=reverse -f bam -m intersection-strict. Read counts were converted to transcripts per million (TPM) and used for cell number-normalization. The expression of all RefSeq genes and ERCC probes was floored at 0.01 and a pseudocount of 0.1 was added to all entries. Values were normalized by equilibrating the expression of the ERCC probes among experiments using normalize.loess from the affy R package.

The ERCC-normalized expression of each gene after 24 h of either A485 or JQAD1 treatment was compared against its expression in dimethyl sulfoxide (DMSO) treated samples to create two-fold changes. These data were then analyzed by gene set enrichment analysis (GSEA) using the gene ontology hallmarks (H) collection in MSigDB to determine relative enrichment on apoptotic hallmark gene sets in JQAD1 vs A485 treated cells (The Gene Ontology Consortium, Nucleic Acids Res 43:D1049-56 (2015); Subramanian et al. Proc. Natl. Acad. Sci. U.S.A. 102:15545-50 (2005)).

For in vivo analyses, tumors were removed from animals treated with either vehicle phosphate-buffered saline (PBS), prior to filtering for single cells through a 0.45 micron filter. Single cell suspensions were then solubilized in TRIzol™ (Ambion) as described above, with processing, including ERCC RNA spike in controls, treatment with RNAse-free DNAse I, and spin purification. Following preparation, there was sufficient material to proceed with RNAseq analysis for four vehicle control and three JQAD1 treated tumor specimen. Purified RNA samples were subjected to library construction with a low input RNA protocol followed by poly-adenylation preparation and sequencing using the Illumina NextSeq® 500 (paired end, 75 bp reads).

Raw reads for RNA-seq of in vivo models were aligned first using hisat2 v2.1 in paired-end mode against the mm9 revision of the mouse genome to filter out contaminating mouse reads. Remaining reads were aligned to a reference genome containing the hg19 revision of the human reference and the sequences of ERCC spike-in probes. Expression was quantified using sorted BAMs, a gene reference built using ERCC sizes and RefSeq genes downloaded Jul. 15, 2017, and htseq-count with parameters -I gene_id -stranded=reverse -f bam -m intersection-strict. Read counts were converted to transcripts per million (TPM) and used for cell number-normalization. The expression of all RefSeq genes and ERCC probes was floored at 0.01 and a pseudocount of 0.1 was added to all entries. Values were normalized by equilibrating the expression of the ERCC probes among experiments using normalize.loess from the affy R package.

Genes were then annotated as either controlled by super-enhancers (n=671) or typical enhancers (n=27116) using H3K27ac data derived from Durbin et al. Nat. Genet. 50:1240-6 (2018), Oldridge et al. Nature 528:418-21 (2015), and Wang et al. Nat. Commun. 10:5622 (2019), and available under GEO database accession number GSE94822. For annotation of gene identity as “transcription factor,” the list of 1639 high-confidence human transcription factors were obtained from Lambert et al. Cell 175:598-9 (2018) and used to annotate RNAseq data. Data was compared by ANOVA with multiple hypothesis testing using the original method of Benjamini and Hochberg, comparing ERCC-controlled RNAseq expression in JQAD1-treated samples against vehicle-treated controls (Benjamini and Hochberg, Stat. Soc, Series B 57:289-300 (1995)).

### Example 10: Biotin-JQAD1 Pulldown Assays

Biotin-JQAD1, synthesized below, was added to 500 μg of whole Kelly cell lysate prepared in immunoprecipitation (IP) lysis buffer (Pierce™ Biotechnology), and incubated for 16 h at 4° C. with end-over-end mixing. One hundred μL of high-capacity streptavidin agarose resin (Pierce™ Biotechnology) was packed into a 1.5 mL Eppendorf tube®, washed three times with cold PBS, prior to addition of cell lysate. Lysate was incubated at room temperature for 10 minutes prior to centrifugation and washing, followed by elution in NuPAGE™ LDS sample buffer with reducing agent (Thermo-Fisher Scientific). Samples were processed by sodium dodecyl sulphate-polyacrylamide gel electrophoresis (SDS-PAGE) as above and blotted using anti-EP300, anti-CRBN, or anti-CBP antibodies.

### Example 11: AlphaLISA® Assay

Assays were performed with minimal modifications from the manufacturer's protocol (PerkinElmer®, USA). Briefly, a 2× solution of components with final concentrations of CRBN-DDB1 at 50 nM, Ni-coated Acceptor Beads at 20 μg/ml, and 15 nM biotinylated-pomalidomide were added in 104 to 384-well plates (AlphaPlate-384, PerkinElmer®, USA). 100 nL of compound in DMSO from stock plates were added by pin transfer using a Janus Workstation (PerkinElmer®, USA). Streptavidin-coated donor beads (20 μg/ml final) were added to the solution, followed by incubation at room temperature for 1 hour, and reading on an Envision 2104 (PerkinElmer®, USA), by the manufacturer's protocol.

### Example 12: Public Database Expression Analysis

Analyses of publicly available expression datasets was performed using either the R2 database or the DepMap portal. Cancer cell line encyclopedia analyses of RNA expression and proteomic expression were performed using the 20Q1 data release (Ghandi et al. Nature 569:503-8 (2019); Nusinow et al. Cell 180:387-402 (2020)). R2 database analyses were performed using the Kocak neuroblastoma dataset of n=649 primary tumor samples (Kocak et al. Cell Death Dis. 4:e586 (2013)).

### Example 13: Motif Enrichment Analysis

CBP and EP300 ChIP-Seq peaks in Kelly and BE2C cells were compared in order to remove the peaks bound by both factors. For each cell line, regions uniquely enriched in P300 or CBP were determined using bedtools intersect -v -f 0.5 -r, which filters regions sharing 50% or more between factors. These unique ChIP-seq peaks were: 7924 of the 9274 peaks for EP300 and 717 of the 2160 peaks for CBP in Kelly cells; 5679 of the 8645 peaks for EP300 and 666 of the 3732 for CBP in BE2C cells. In each subset, a motif enrichment analysis was performed as described in Mariani et al. Cell Syst. 5:187-201 (2017). Briefly, the 500 highest confidence ChIP-seq narrow peaks as evaluated by the FDR from the peak calling were identified and trimmed to 200 bp around the peak summit. A background set of 500 200-bp sequences, each corresponding to a trimmed peak, was generated using GENRE software with the default human setting (promoter overlap, repeat overlap, GC content, CpG dinucleotide frequency). A collection of 44 position weight matrices (PWMs) was manually curated as a representative repertoire of sequence-specific transcription factor (TF) families, including motifs associated to previously determined master transcription factors in neuroblastoma cell lines (GATA3, TFAP2β, ISL1, MEIS2, PHOX2B, TCF3, and TWIST2). By comparing the trimmed peaks and the associated background sequences, TF motif enrichment was quantified using a well-established area under the receiver operator curve (AUROC)-based metric that assesses the presence of a TF motif among the 500 highest confidence peaks (foreground set) as compared to a background set of sequences (Gordan et al. Genome Res. 19:2090-2100 (2009)). For the AUROC quantification, TF ChIP-seq data were analyzed as described in Mari ani et al. Cell Sy st. 5:187-201 (2017) (http://thebrain.bwh.harvard.edu/glossary-GENRE/download. html), which includes the use of the R-function “matchPWM” (R-package “Biostrings”) to score each PWM against each sequence and the evaluation of an adjusted p-value to ensure statistical significance. In both cell lines, TFAP2β (PWM M5912_1 from CISBP databank Version 1.02) was the only PWM that showed a relevant differential enrichment, namely an enrichment above 0.6 AUROC (p-value <0.001) in EP300-unique peaks, and no enrichment (AUROC ˜0.5, p-value>0.1) in CBP unique peaks.

### Example 14: Profiling Relative Inhibition Simultaneously in Mixtures (PRISM) Screening

PRISM barcoded pooled screening was performed using JQAD1 in 578 barcoded cell lines as described in Corsello et al. Nat. Med. 23:405-8 (2017) and Corsello et al. Nat Cancer 1:235-48 (2020). Some cell lines included in the screen were genetically engineered to express exogenous genes, and these cell lines were removed to yield 557 cell lines. Briefly, cells in pools of 20-25 were thawed and plated into 384-well plates (1250 cells/well for adherent cell pools, 2000 cells/well for suspension or mixed suspension/adherent cell pools) containing compound (top concentration: 10 μM, 8-point, threefold dilution). All conditions were tested in triplicate. Cells were lysed after 5 days of treatment and mRNA-based Luminex® detection of barcode abundance from lysates was carried out as described in Corsello et al. Cancer 1:235-48 (2020). Luminex median fluorescence intensity (MFI) data was input to a standardized R pipeline to generate viability estimates relative to vehicle treatment for each cell line and treatment condition, and to fit dose-response curves from viability data.

### Example 15: STRING Database Analysis

Neuroblastoma-specific genetic dependencies (n=146) were identified in Durbin et al. Nat. Genet. 50:1240-6 (2018). Dependency genes were intersected with the Gene Ontology term “Cellular Component — nucleus” to derive the list of nuclear factor-encoding dependency genes (n=84) (The Gene Ontology Consortium, Nucleic Acids Res. 43:D1049-56 (2015); Mi et al. Nat. Protoc. 8:1551-66 (2013)). These genes were input into the String database to generate interaction plots using medium confidence interaction scores and hiding unlinked nodes. Network edges reflect evidence of interactions (Szklarczyk et al. Nucleic Acids Res. 43:D447-52 (2015)). Color indicates commercially available compounds targeting the protein (red=yes, grey=no).

### Example 16: Genome-wide Occupancy Analysis

ChIP-seq was performed as previously described for cell lines (Durbin et al. Nat. Genet. 50:1240-6 (2018)). The following antibodies were used for ChIP: EP300 (Abcam, ab10485), CBP (#7389, Cell Signaling Technology®), TFAP2β (#2509, Cell Signaling Technology®), ASCL1 (sc-374104, Santa Cruz Biotechnology) and H3K27ac (Abcam ab4729). For each ChIP, 10 μg of antibody was added to 3 ml of sonicated nuclear extract. Illumina® sequencing, library construction, and ChIP-seq analysis methods were performed as described in Mansour et al. Science 346, 1373-7 (2014) and Sanda et al. Cancer Cell 22:209-21 (2012). Remaining ChIP-seq and assay for transposase-accessible chromatin (ATAC)-sequencing data were extracted from previously published datasets (GSE120074, GSE94822, GSE65664) available through the GEO portal. For experiments involving analysis of quantitative changes in H3K27ac, pellets of neuroblastoma cells were externally spiked in with similarly fixed and processed S2 cells at 1:10 ratio, prior to sonication.

### Example 17: Cell Line ChIP-seq and ATAC-Seq Processing and Display

Reads were aligned to the human genome (hg19) using bowtie with parameters -k 2 -m 2 -e 70 -best and -1 set to the read length. For visualization, WIG files were created from aligned ChIP-seq read positions using MACS 1.4 with parameters -w -S -space=50 -nomodel -shiftsize=200 to artificially extend reads to be 200 bp and to calculate their density in 50 bp bins. Read counts in 50 bp bins were then normalized to the millions of mapped reads, giving reads per million (rpm) values. Locus-specific visualization was performed using IGV 2.4.10. (Broad Institute).

### Example 18: ChIP-seq Enriched Regions

Regions enriched in ChIP-seq signal were identified using MACS 1.4.2 with corresponding control and parameters -keep-dup=auto and -p 1 e-9. Regions displayed in FIG. 2B, FIG. 2C, FIG. 9A, and FIG. 9B were created from the collapsed union of master transcription factor (HAND2, ISL1, PHOX2B, GATA3, TBX2, ASCL1, TFAP2β) peaks from the respective cell line.

### Example 19: ChIP-seq Coverage Heatmaps

ChIP-seq and ATAC-Seq signal was quantified for heatmap display in 4 kb windows centered on the middle of each collapsed peak using bamToGFF with parameters -m 50 -r -f 1. Rows were ordered by either MYCN signal in the whole displayed window (FIGS. 2C and 9B) or the ratio of EP300 to CBP. EP300/CBP ratio was calculated in 500bp windows centered on the middle of each collapsed master TF binding site using bamToGFF with parameters -m 1 -r.

### Example 20: ChIP-RX Alignment and Processing

ChIP-RX reads from Kelly cells treated with 500 nM JQAD1 were aligned in multiple steps. Reads were aligned to the dm6 revision of the D. melanogaster reference genome with -k 1 —chunkmbs 256—best to identify spiked-in DNA. Counts of fly reads were determined by counting unique read names in the aligned read file. Remaining non-fly reads were aligned to the hg19 revision of the human reference genome with parameters -k 2 -m 2 -chunkmbs 256 -best -175. Visualization files were constructed using macs 1.4 with parameters -w -S -space=50 -nomodel -shiftsize=200 to generate wiggle files, which were subsequently normalized by the millions of fly-mapped reads in the corresponding sample.

### Example 21: Super-Enhancer and Typical Enhancer Identification and Assignment

Super-enhancers in Kelly xenografts were identified using ROSE and the single-end BAMs generated as described above (Mansour et al. Science 346:1373-7 (2014)). Briefly, two sets of peaks of H3K27ac were identified using MACS with parameter sets -keep-dup=auto -p 1e-9 and -keep-dup=all -p 1e-9. Identified peaks that contact the region chr2:14817188-17228298 were discarded because they fall within the genomically amplified regions around MYCN, as described in Durbin et al. Nat. Genet. 50:1240-6 (2018)). The collapsed union of regions called using both MACS parameter sets that do not contact the discarded MYCN-proximal region were used as input for ROSE, as described in Mansour et al. Science 346:1373-7 (2014), with some modifications. H3K27ac peaks were stitched computationally if they were within 12500 bp of each other, though peaks fully contained within +/−2000 bp from a RefSeq promoter were excluded from stitching. These stitched enhancers were ranked by their H3K27ac signal (length*density) with input signal subtracted. Super-enhancers were defined geometrically as those enhancers above the point at which the line y=x is tangent to the curve. Stitched enhancers (typical enhancers and super-enhancers) were assigned to the single active gene whose transcription start site is nearest the center of the stitched enhancer. Active genes were determined by taking the top two-thirds of all RefSeq promoters (+/−500 bp) ranked by their H3K27ac signal. H3K27ac signal in promoters was determined using bamToGFF with parameters -e 200 -m 1 -r -d.

H3K27ac ChIP-RX read coverage of stitched enhancers was quantified using bamToGFF with parameter -t TRUE and divided by the millions of mapped reads, from which read-per-million values from the corresponding input experiment was subtracted. These values were used to create fold-changes during the treatment time-course.

### Example 22: DepMap Dependency Analysis

Analysis of dependency data was retrieved from the DepMap portal using the 20Q2 dataset. Dependency data were extracted as probability of dependency for all cell lines (n=757), for the two genes EP300 and CBP. Cell lines were annotated to lineages as described by the DepMap portal. Specific dependency in neuroblastoma cell lines was identified by extracting the probability of dependency on EP300 or CBP across 19 neuroblastoma cell lines (SIMA, KPNYN, SKNDZ, SKNFI, CHP212, NB1, LS, Kelly, COGN305, COGN278, SKNBE2, LAN2, SKNAS, NGP, IMR32, GIMEN, NB1643, MHHNB11, CHLA15) and comparing with probability of dependency >0.5 indicating a cell line likely to be dependent on the denoted gene (Meyers et al. Nat. Genet. 49:1779-84 (2017); Oberlick et al. Cell Rep 28:2331-44 (2019)). For analysis across all tumor cell lines (FIG. 7A-FIG. 7B), the average and standard deviation of the probability of dependency was used as a continuous metric. Details of individual cell lines are available in the DepMap Portal. Lineages and number of cell lines in the 20Q2 DepMap release are: acute myeloid leukemia (AML) (n=20), B-cell leukemia (n=11), lymphoma (n=19), bile duct/gallbladder cancer (n=29), bladder carcinoma (n=29), breast cancer (n=34), cervical cancer (n=12), sarcoma not otherwise specified (NOS) (n=8-chondrosarcoma (n=1), epithelioid sarcoma (n=1), fibrosarcoma (n=2), leiomyosarcoma (n=1), pleomorphic sarcoma (n=1), thyroid sarcoma (n=1), undifferentiated sarcoma (n=1)), chordoma (n=3), chronic myeloid leukemia (CML) (n=7), colorectal carcinoma (n=37), endometrial/uterine carcinoma (n=26), esophageal carcinoma (n=23), Ewing sarcoma (n=15), eye cancer (n=4), gastric carcinoma (n=26), glioblastoma (n=33), glioma (n=17), head and neck carcinoma (n=32), kidney carcinoma (n=21), liposarcoma (n=5), liver carcinoma (n=22), lung carcinoma (n=106), medulloblastoma (n=8), melanoma (n=41), multiple myeloma (n=20), neuroblastoma (n=19), osteosarcoma (n=8), ovarian carcinoma (n=42), pancreatic carcinoma (n=34), rhabdoid tumors (n=7), rhabdomyosarcoma (n=11), squamous cell carcinoma (n=4), synovial sarcoma (n=5), T-acute leukemia (n=3), and thyroid carcinoma (n=6). Data from lineages with fewer than 3 cell lines were removed from the analysis.

### Example 23: Synthesis of JQAD1

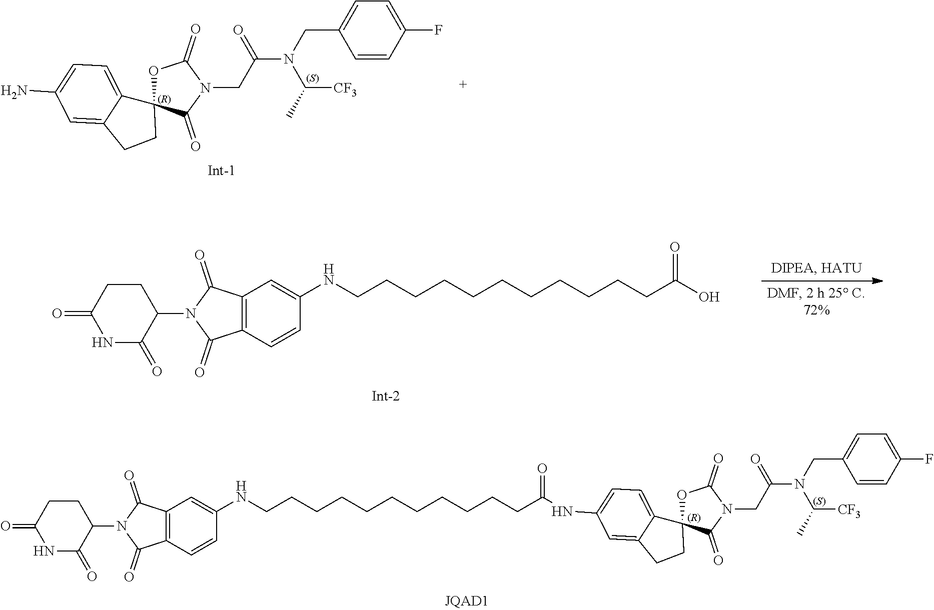

Scheme 1: Synthesis of JQAD1 (12-((2-(2,6-dioxopiperidin-3-yl)-1,3-dioxoisoindolin-5-yl)amino)-N-((R)-3′-(2-((4-fluorobenzyl)((S)-1,1,1- trifluoropropan-2-yl)amino)-2-oxoethyl)-2′,4′-dioxo-2,3-dihydrospiro[indene-1,5′-oxazolidin]-5-yl)dodecanamide). Compounds Int-1 and Int-2 were synthesized according to Michaelides et. al., ACS Med. Chem. Lett. 9:28-33 (2018) and International Patent Publication WO2020/006157 A1. To a mixture of Int-1 (500 mg, 1.04 mmol, 1.0 eq.) and Int-2 (492 mg, 1.04 mmol, 1 eq.) in N,N-dimethylformamide (DMF, 10 mL, 0.1 M) in a 50-mL flask, N,N-diisopropylethylamine (DIPEA) (349 μL, 2.09 mmol, 2 eq.) and hexafluorophosphate azabenzotriazole tetramethyl uronium (HATU) (793 mg, 2.09 mmol, 2 eq.) were added. The reaction mixture was stirred at 25° C. for 2 h. After the reaction was complete, the mixture was purified directly by silica gel chromatography (Ethyl Acetate/Hexane, 20-90% gradient), and the solvent was removed under reduced pressure to give JQAD1 as yellow powder (700 mg, 72% yield).

¹HNMR (500 MHz, Acetone-d₆) i 9.90; (d, J=4.0 Hz, 1H), 9.26; (d, J=5.1 Hz, 1H), 7.90; (d, J=7.7 Hz, 1H), 7.59; (td, J=7.8, 2.7 Hz, 1H), 7.49; (d, J =9.7 Hz, 3H), 7.36-7.31; (m, 1H), 7.22; (t, J=8.6 Hz, 2H), 7.13-7.00; (m, 3H), 6.42; (d, J=5.9 Hz, 1H), 5.51; (p, J=7.8 Hz, 1H), 5.07; (ddd, J=12.1, 7.6, 4.2 Hz, 2H), 4.97-4.81; (m, 1H), 4.67; (dd, J=71.1, 16.7 Hz, 1H), 4.43; (dd, J=90.6, 16.6 Hz, 1H), 3.38; (q, J=6.3 Hz, 2H), 3.28-3.04; (m, 2H), 3.03-2.85; (m, 3H), 2.85-2.70; (m, 4H), 2.56; (dddd, J=14.5, 12.1, 8.6, 4.2 Hz, 1H), 2.39; (t, J=7.2 Hz, 2H), 2.27-2.17; (m, 1H), 2.07; (p, J=2.2 Hz, 2H), 1.73-1.67; (m, 4H), 1.39; (dd, J=38.4, 5.3 Hz, 12H).

MS (ESI) calculated. For C₄₈H₅₂F₄N₆O₉: 932.37, Found: [M+1] 933.36.

### Example 24: Synthesis of Biotin-JQAD1

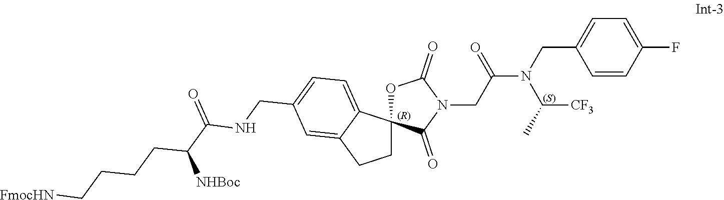

Int-3 ((9H-fluoren-9-yl)methyl tert-butyl (6-((((R)-3′-(2-((4-fluorobenzyl)((S)-1,1,1-trifluoropropan-2-yl)amino)-2-oxoethyl)-2′,4′-dioxo-2,3- dihydrospiro[indene-1,5′-oxazolidin]-5 -yl)methyl)amino)-6-oxohexane-1,5-diyl)dicarbamate)

To a solution of Int-1(20.0 mg, 0.042 mmol, 1 eq.), Boc-Lys(Fmoc)-OH (19.7 mg, 0.042 mmol, 1 eq.) in DMF (3 mL, 0.14M), DIPEA (14.0 4, 0.084 mmol, 2 eq.) and HATU (31.9 mg, 0.084 mmol, 2 eq.) were added. The reaction mixture was stirred at 25° C. for 2 h. After the reaction was complete, the mixture was purified directly by silica gel chromatography (Ethyl Acetate/Hexane, 20-90% gradient), and the solvent was removed under reduced pressure to give int-3 as yellow powder (33.7 mg, 85% yield).

MS (ESI) calculated. For C₅₀H₅₃F₄N₅O₉: 943.38, Found: [M+1]944.39.

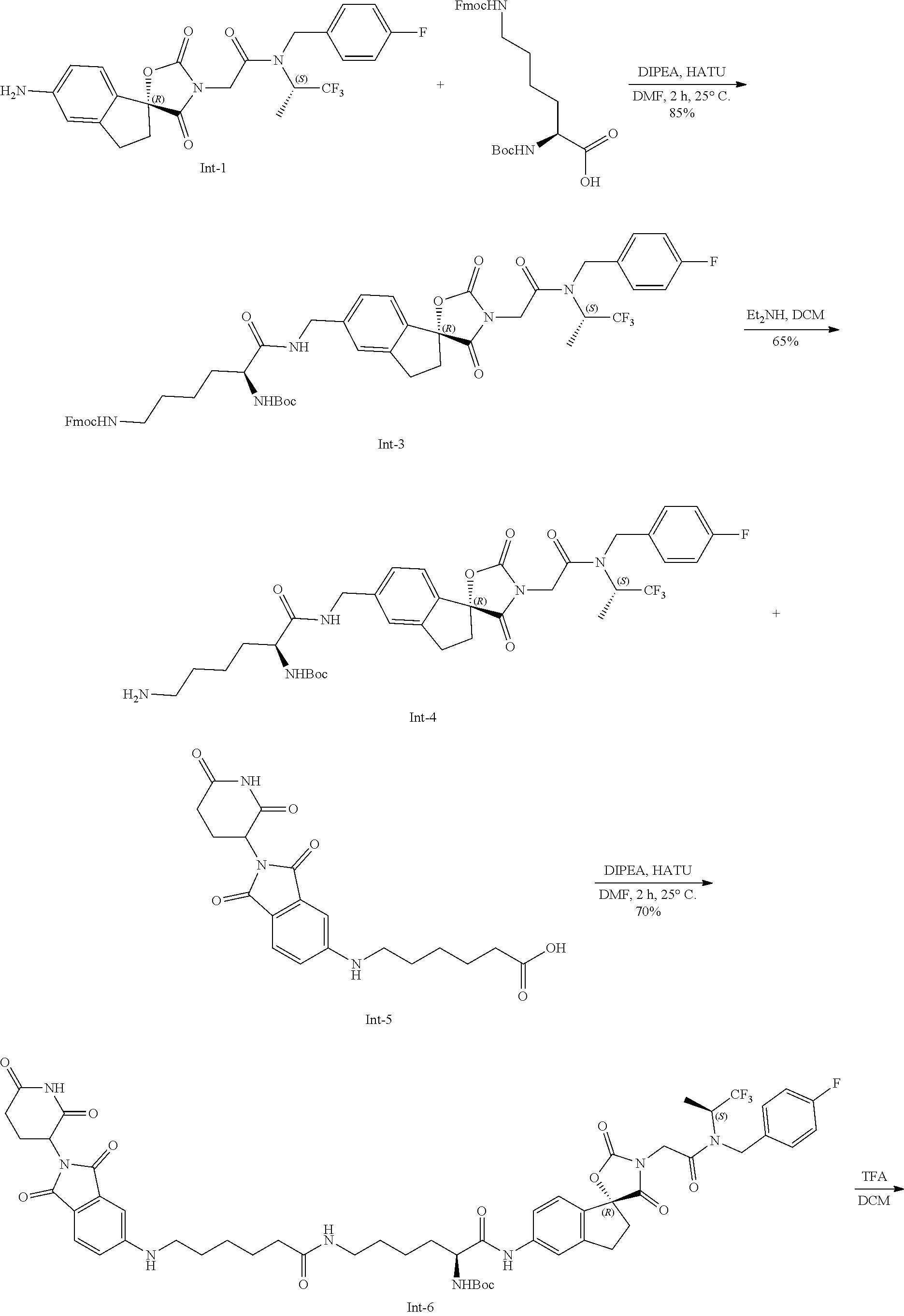

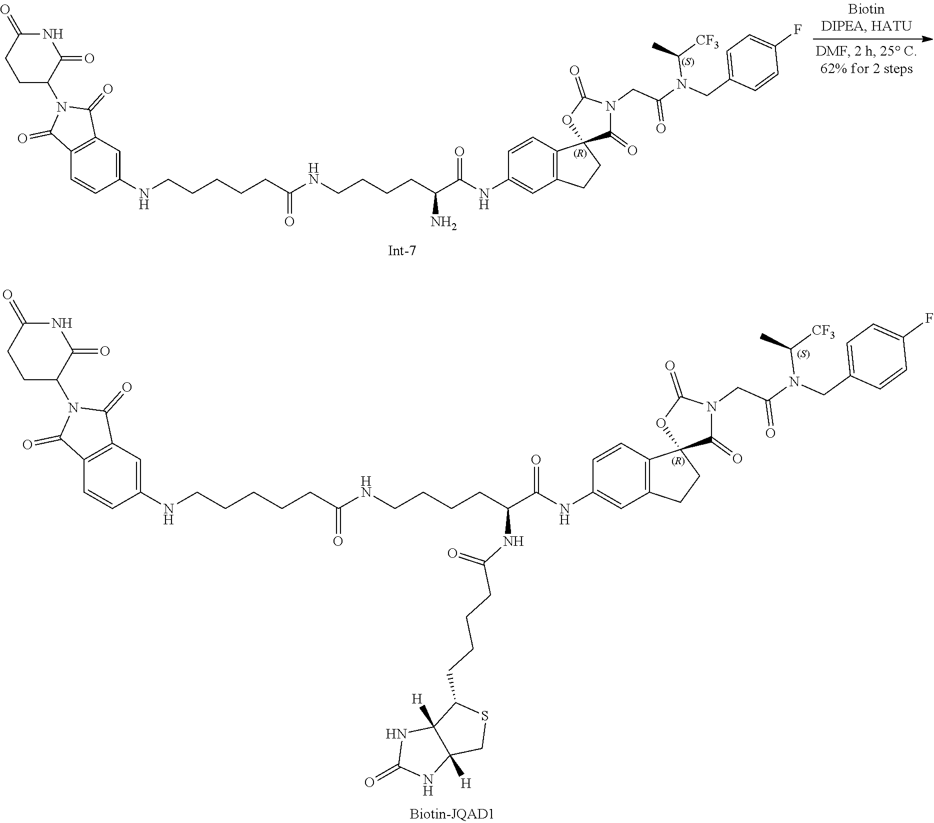

Scheme 2: Synthesis of Biotin-JQAD1 (6-(6-((2-(2,6-dioxopiperidin-3-yl)-1,3-dioxoisoindolin-5-yl)amino)hexanamido)-N-(®-3′-(2-((4- fluorobenzyl)((S)-1,1,1-trifluoropropan-2-yl)amino)-2-oxoethyl)-2′,4′-dioxo-2,3-dihydrospiro[indene-1,5′-oxazolidin]-5-yl)-2-(5-((3aS,4S,6aR)-2-oxohexahydro-1H-thieno[3,4-d]44midazole-4-yl)pentanamido)hexanamide)

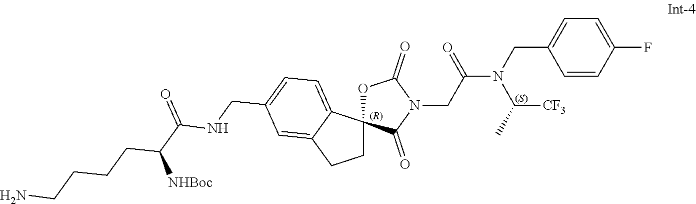

Int-4 (tert-Butyl (6-amino-1-((((R)-3′-(2-((4-fluorobenzyl)((S)-1,1,1-trifluoropropan-2-yl)amino)-2-oxoethyl)-2′,4′-dioxo-2,3-dihydrospiro[indene-1,5′-oxazolidin]-5 -yl)methyl)amino)-1-oxohexan-2-yl)carbamate)

To a solution of Int-3 (33.7 mg, 0.036 mmol) in dichloromethane (DCM) (2 mL, 0.018M), diethyl amine (1 mL) was added dropwise. The reaction was stirred at 25° C. for 1 h. The solvent was removed under reduced pressure and the resulting residue was purified by silica gel chromatography (MeOH/DCM, 0-10% gradient). The solvent was removed under reduced pressure to give Int-4 as colorless oil (25.4 mg, 95% yield).

MS (ESI) calculated. For C₃₅H₄₃F₄N₅O₇: 721.31, Found: [M+1]722.35.

Int-5 was synthesized according to International Patent Publication WO2020/006157 A1.

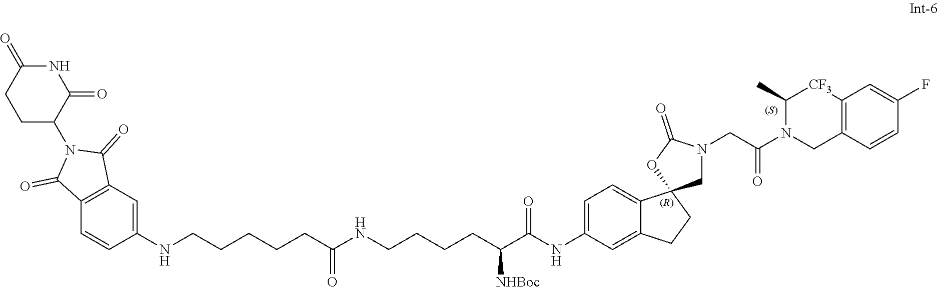

Int-6 (tert-Butyl (6-(6-((2-(2,6-dioxopiperidin-3-yl)-1,3-dioxoisoindolin-5-yl)amino)hexanamido)-1-(((R)-3′-(2-((4-fluorobenzyl)((S)-1,1,1- trifluoropropan-2-yl)amino)-2-oxoethyl)-2′,4′-dioxo-2,3-dihydrospiro[indene-1,5′-oxazolidin]-5-yl)amino)-1-oxohexan-2-yl)carbamate)

To a solution of Int-4 (20.0 mg, 0.028 mmol, 1 eq.) and Int-5 (11.9 mg, 0.028 mmol, 1 eq.) in DMF (2 mL, 0.014M), DIPEA (9.33 μL, 0.056 mmol, 2 eq.), HATU (21.3 mg, 0.084 mmol, 2 eq.) were added. The reaction mixture was stirred at 25° C. for 2 h. After the reaction was complete, the mixture was purified by silica gel chromatography (MeOH/DCM, 0-10% gradient), and the solvent was removed under reduced pressure to give Int-6 as yellow oil (21.1 mg, 70% yield).

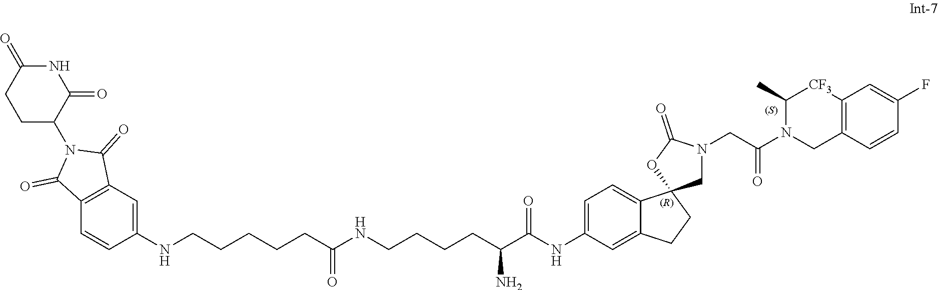

Int-7 (2-Amino-6-(6-((2-(2,6-dioxopiperidin-3-yl)-1,3-dioxoisoindolin-5-yl)amino)hexanamido)-N-((R)-3′-(2-((4-fluorobenzyl)((S)-1,1,1- trifluoropropan-2-yl)amino)-2-oxoethyl)-2′,4′-dioxo-2,3-dihydrospiro[indene-1,5′-oxazolidin]-5-yl)hexanamide)

To a solution of Int-6 (21.1 mg, 0.020 mmol) in DCM (2 mL, 0.01 M), trifluoroacetic acid (TFA) (1 mL) was added dropwise. The reaction was stirred at 25° C. for 1 h, and the solvent was removed under reduced pressure. The resulting residue was subjected to the next step reaction without further purification.

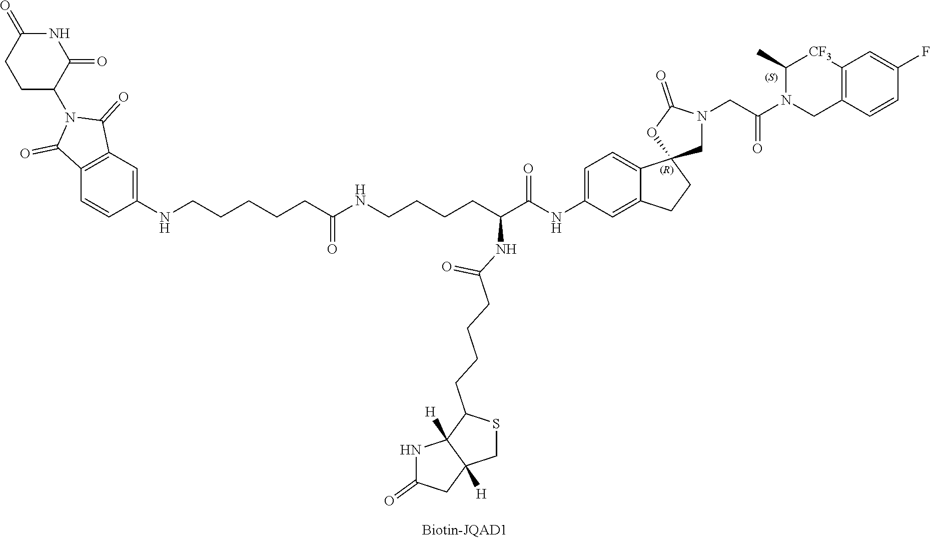

To a solution of Int-7 and biotin (2.45 mg, 0.010 mmol, 1 eq.) in DMF (1 mL, 0.01 M), DIPEA (3.33 μL, 0.020 mmol, 2 eq.) and HATU (7.51 mg, 0.020 mmol, 2 eq.) were added. The resulting mixture was stirred at 25° C. for 2 h. After the reaction was complete, the mixture was purified through silica gel chromatography (MeOH/DCM, 0-10% gradient), and the solvent was removed under reduced pressure to give Biotin-JQAD1 as yellow oil (5.3 mg, 52% yield).

MS (ESI) calculated. For C₅₈H₆₆F₄N₁₀O₁₂S: 1202.45, Found: [M+1]1023.48.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts, and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of treating a subject with a disease or disorder associated with E1A-binding protein P300 (EP300) dependency comprising:
obtaining a test sample from a subject having or at risk of developing the disease or disorder;
identifying an increased expression level of cereblon (CRBN) in the test sample as compared to the expression level of CRBN in a reference sample; and
administering to the subject a therapeutically effective amount of a selective degrader of EP300.

2. The method of claim 1, wherein the test sample is obtained from a tumor tissue or a tumor microenvironment: or wherein the test sample is obtained from a bodily fluid selected from the group consisting of plasma, blood, urine, sputum, and cerebrospinal fluid (CSF); or wherein the reference sample is obtained from healthy normal tissue or tumor tissue.

3. (canceled)

4. (canceled)

5. The method of claim 2, wherein the reference sample is obtained from healthy normal tissue from the same individual as the test sample or one or more healthy normal tissues from different individuals.

6. The method of claim 1, wherein the selective degrader of EP300 is JQAD1: 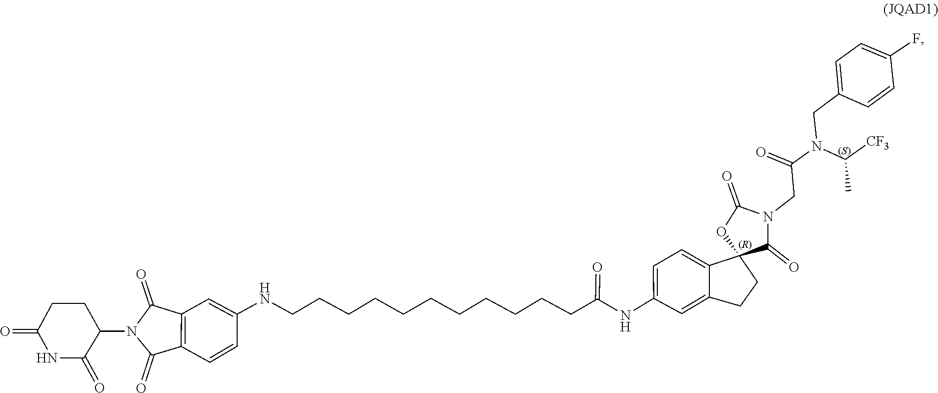
or a pharmaceutically acceptable salt thereof.

7. The method of claim 1, wherein the disease or disorder is an EP300-dependent cancer.

8. The method of claim 7, wherein the cancer comprises a solid tumor; or wherein the cancer is a hematologic cancer.

9. The method of claim 8, wherein the solid tumor is neuroblastoma, rhabdomyosarcoma, melanoma, colon cancer, rectum cancer, stomach cancer, breast cancer, brain cancer, or pancreatic cancer; or
wherein the hematologic cancer is leukemia, myeloma, or lymphoma.

10. The method of claim 9, wherein the neuroblastoma is high-risk neuroblastoma.

11. (canceled)

12. (canceled)

13. The method of claim 7, wherein tumor cell survival, tumor cell proliferation, or tumor metastasis is inhibited, or wherein tumor cell growth is reduced or wherein tumor cell apoptosis is induced.

14. (canceled)

15. The method of claim 1, further comprising administering to the subject a chemotherapeutic agent, radiation therapy, cryotherapy, hormone therapy, immunotherapy, or stem cell transplant; or
further comprising administering to the subject a combination chemotherapy agent or
further comprising administering to the subject an immunosuppressant agent.

16. The method of claim 15, wherein the chemotherapeutic agent comprises cis-retinoic acid, cyclophosphamide, cisplatin, carboplatin, vincristine, doxorubicin, etoposide, topotecan, busulfan and melphalan, or thiotepa; or wherein the chemotherapeutic agent is administered with a steroid.

17. (canceled)

18. The method of claim 16, wherein the steroid comprises prednisone or dexamethasone.

19. (canceled)

20. The method of claim 15, wherein the combination chemotherapy agent comprises carboplatin or cisplatin, cyclophosphamide, doxorubicin, and etoposide, or irinotecan, temozolomide, or ifosfamide.

21. (canceled)

22. The method of claim 15, wherein the immunosuppressant agent comprises dinutuximab with or without cis-retinoic acid.

23. The method of claim 1, wherein the subject is a human.

24. The method of claim 1, wherein the therapeutically effective amount of the selective degrader of EP300 or a pharmaceutically acceptable salt thereof, is administered orally to a subject in the form of a tablet.

25. The method of claim 1, wherein the therapeutically effective amount of the selective degrader of EP300 or a pharmaceutically acceptable salt is administered orally to the subject in the form of a capsule.

26. The method of claim 1, wherein the therapeutically effective amount of the selective degrader of EP300 or a pharmaceutically acceptable salt is administered parenterally to the subject in the form of a liquid.

27. A method of determining whether EP300 degradation in a subject with cancer will result in clinical benefit in the subject comprising:
obtaining a test sample from a subject having or at risk of developing cancer;
determining the expression level of CRBN in the test sample;
comparing the expression level of CRBN in the test sample with the expression level of CRBN in a reference sample; and
determining whether EP300 degradation will inhibit the cancer in the subject if the expression level of CRBN in the test sample differs from the expression level of the CRBN in the reference sample.

28. The method of claim 27, wherein the test sample is obtained from a tumor tissue or a tumor microenvironment; or wherein the test sample is obtained from a bodily fluid.

29. (canceled)

30. The method of claim 28, wherein the bodily fluid is selected from the group consisting of plasma, blood, urine, sputum, and CSF; or
wherein the reference sample is obtained from healthy normal tissue.

31. (canceled)

32. The method of claim 27, wherein clinical benefit in the subject comprises complete or partial response as defined by response evaluation criteria in solid tumors (RECIST), stable disease as defined by RECIST, or long-term survival in spite of disease progression or response as defined by irRC criteria; or
wherein the test sample is obtained from the cancer, and further comprising determining that EP300 degradation in a subject with cancer will result in clinical benefit in the subject if the expression level of CRBN in the test sample is equal to or higher than the level of CRBN in the reference sample; or
wherein the test sample is obtained from the cancer, and further comprising determining that EP300 degradation in a subject with cancer will not result in clinical benefit in the subject if the expression level of CRBN in the test sample is lower than the level of CRBN in the reference sample.

33. (canceled)

34. (canceled)


SEQUENCE LISTING (6 sequences)

SEQ ID NO: 1 - protein, 2388 aa, Homo sapiens
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSLGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSSLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 2 - DNA, 8701 bp, Homo sapiens
gagaaggaggaggacagcgccgaggaggaagaggttgatggcggcggcggagctccgagagacctcggctgggcaggggccggccgtggcgggccggggactgcgcctctagagccgcgagttctcgggaattcgccgcagcggacgcgctcggcgaatttgtgctcttgtgccctcctccgggcttgggcccaggcccggcccctcgcacttgcccttaccttttctatcgagtccgcatccctctccagccactgcgacccggcgaagagaaaaaggaacttcccccaccccctcgggtgccgtcggagccccccagcccacccctgggtgcggcgcggggaccccgggccgaagaagagatttcctgaggattctggttttcctcgcttgtatctccgaaagaattaaaaatggccgagaatgtggtggaaccggggccgccttcagccaagcggcctaaactctcatctccggccctctcggcgtccgccagcgatggcacagattttggctctctatttgacttggagcacgacttaccagatgaattaatcaactctacagaattgggactaaccaatggtggtgatattaatcagcttcagacaagtcttggcatggtacaagatgcagcttctaaacataaacagctgtcagaattgctgcgatctggtagttcccctaacctcaatatgggagttggtggcccaggtcaagtcatggccagccaggcccaacagagcagtcctggattaggtttgataaatagcatggtcaaaagcccaatgacacaggcaggcttgacttctcccaacatggggatgggcactagtggaccaaatcagggtcctacgcagtcaacaggtatgatgaacagtccagtaaatcagcctgccatgggaatgaacacagggatgaatgcgggcatgaatcctggaatgttggctgcaggcaatggacaagggataatgcctaatcaagtcatgaacggttcaattggagcaggccgagggcgacagaatatgcagtacccaaacccaggcatgggaagtgctggcaacttactgactgagcctcttcagcagggctctccccagatgggaggacaaacaggattgagaggcccccagcctcttaagatgggaatgatgaacaaccccaatccttatggttcaccatatactcagaatcctggacagcagattggagccagtggccttggtctccagattcagacaaaaactgtactatcaaataacttatctccatttgctatggacaaaaaggcagttcctggtggaggaatgcccaacatgggtcaacagccagccccgcaggtccagcagccaggcctggtgactccagttgcccaagggatgggttctggagcacatacagctgatccagagaagcgcaagctcatccagcagcagcttgttctccttttgcatgctcacaagtgccagcgccgggaacaggccaatggggaagtgaggcagtgcaaccttccccactgtcgcacaatgaagaatgtcctaaaccacatgacacactgccagtcaggcaagtcttgccaagtggcacactgtgcatcttctcgacaaatcatttcacactggaagaattgtacaagacatgattgtcctgtgtgtctccccctcaaaaatgctggtgataagagaaatcaacagccaattttgactggagcacccgttggacttggaaatcctagctctctaggggtgggtcaacagtctgcccccaacctaagcactgttagtcagattgatcccagctccatagaaagagcctatgcagctcttggactaccctatcaagtaaatcagatgccgacacaaccccaggtgcaagcaaagaaccagcagaatcagcagcctgggcagtctccccaaggcatgcggcccatgagcaacatgagtgctagtcctatgggagtaaatggaggtgtaggagttcaaacgccgagtcttctttctgactcaatgttgcattcagccataaattctcaaaacccaatgatgagtgaaaatgccagtgtgccctccctgggtcctatgccaacagcagctcaaccatccactactggaattcggaaacagtggcacgaagatattactcaggatcttcgaaatcatcttgttcacaaactcgtccaagccatatttcctacgccggatcctgctgctttaaaagacagacggatggaaaacctagttgcatatgctcggaaagttgaaggggacatgtatgaatctgcaaacaatcgagcggaatactaccaccttctagctgagaaaatctataagatccagaaagaactagaagaaaaacgaaggaccagactacagaagcagaacatgctaccaaatgctgcaggcatggttccagtttccatgaatccagggcctaacatgggacagccgcaaccaggaatgacttctagtttgaatcaatttggccagatgagcatggcccagccccctattgtaccccggcaaacccctcctcttcagcaccatggacagttggctcaacctggagctctcaacccgcctatgggctatgggcctcgtatgcaacagccttccaaccagggccagttccttcctcagactcagttcccatcacagggaatgaatgtaacaaatatccctttggctccgtccagcggtcaagctccagtgtctcaagcacaaatgtctagttcttcctgcccggtgaactctcctataatgcctccagggtctcaggggagccacattcactgtccccagcttcctcaaccagctcttcatcagaattcaccctcgcctgtacctagtcgtacccccacccctcaccatactcccccaagcataggggctcagcagccaccagcaacaacaattccagcccctgttcctacacctcctgccatgccacctgggccacagtcccaggctctacatccccctccaaggcagacacctacaccaccaacaacacaacttccccaacaagtgcagccttcacttcctgctgcaccttctgctgaccagccccagcagcagcctcgctcacagcagagcacagcagcgtctgttcctaccccaacagcaccgctgcttcctccgcagcctgcaactccactttcccagccagctgtaagcattgaaggacaggtatcaaatcctccatctactagtagcacagaagtgaattctcaggccattgctgagaagcagccttcccaggaagtgaagatggaggccaaaatggaagtggatcaaccagaaccagcagatactcagccggaggatatttcagagtctaaagtggaagactgtaaaatggaatctaccgaaacagaagagagaagcactgagttaaaaactgaaataaaagaggaggaagaccagccaagtacttcagctacccagtcatctccggctccaggacagtcaaagaaaaagattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggaccgctttgtctacacctgcaatgaatgcaagcaccatgtggagacacgctggcactgtactgtctgtgaggattatgacttgtgtatcacctgctataacactaaaaaccatgaccacaaaatggagaaactaggccttggcttagatgatgagagcaacaaccagcaggctgcagccacccagagcccaggcgattctcgccgcctgagtatccagcgctgcatccagtctctggtccatgcttgccagtgtcggaatgccaattgctcactgccatcctgccagaagatgaagcgggttgtgcagcataccaagggttgcaaacggaaaaccaatggcgggtgccccatctgcaagcagctcattgccctctgctgctaccatgccaagcactgccaggagaacaaatgcccggtgccgttctgcctaaacatcaagcagaagctccggcagcaacagctgcagcaccgactacagcaggcccaaatgcttcgcaggaggatggccagcatgcagcggactggtgtggttgggcagcaacagggcctcccttcccccactcctgccactccaacgacaccaactggccaacagccaaccaccccgcagacgccccagcccacttctcagcctcagcctacccctcccaatagcatgccaccctacttgcccaggactcaagctgctggccctgtgtcccagggtaaggcagcaggccaggtgacccctccaacccctcctcagactgctcagccaccccttccagggcccccacctgcagcagtggaaatggcaatgcagattcagagagcagcggagacgcagcgccagatggcccacgtgcaaatttttcaaaggccaatccaacaccagatgcccccgatgactcccatggcccccatgggtatgaacccacctcccatgaccagaggtcccagtgggcatttggagccagggatgggaccgacagggatgcagcaacagccaccctggagccaaggaggattgcctcagccccagcaactacagtctgggatgccaaggccagccatgatgtcagtggcccagcatggtcaacctttgaacatggctccacaaccaggattgggccaggtaggtatcagcccactcaaaccaggcactgtgtctcaacaagccttacaaaaccttttgcggactctcaggtctcccagctctcccctgcagcagcaacaggtgcttagtatccttcacgccaacccccagctgttggctgcattcatcaagcagcgggctgccaagtatgccaactctaatccacaacccatccctgggcagcctggcatgccccaggggcagccagggctacagccacctaccatgccaggtcagcagggggtccactccaatccagccatgcagaacatgaatccaatgcaggcgggcgttcagagggctggcctgccccagcagcaaccacagcagcaactccagccacccatgggagggatgagcccccaggctcagcagatgaacatgaaccacaacaccatgccttcacaattccgagacatcttgagacgacagcaaatgatgcaacagcagcagcaacagggagcagggccaggaataggccctggaatggccaaccataaccagttccagcaaccccaaggagttggctacccaccacagcagcagcagcggatgcagcatcacatgcaacagatgcaacaaggaaatatgggacagataggccagcttccccaggccttgggagcagaggcaggtgccagtctacaggcctatcagcagcgactccttcagcaacagatggggtcccctgttcagcccaaccccatgagcccccagcagcatatgctcccaaatcaggcccagtccccacacctacaaggccagcagatccctaattctctctccaatcaagtgcgctctccccagcctgtcccttctccacggccacagtcccagcccccccactccagtccttccccaaggatgcagcctcagccttctccacaccacgtttccccacagacaagttccccacatcctggactggtagctgcccaggccaaccccatggaacaagggcattttgccagcccggaccagaattcaatgctttctcagcttgctagcaatccaggcatggcaaacctccatggtgcaagcgccacggacctgggactcagcaccgataactcagacttgaattcaaacctctcacagagtacactagacatacactagagacaccttgtagtattttgggagcaaaaaaattattttctcttaacaagactttttgtactgaaaacaatttttttgaatctttcgtagcctaaaagacaattttccttggaacacataagaactgtgcagtagccgtttgtggtttaaagcaaacatgcaagatgaacctgagggatgatagaatacaaagaatatatttttgttatggctggttaccaccagcctttcttcccctttgtgtgtgtggttcaagtgtgcactgggaggaggctgaggcctgtgaagccaaacaatatgctcctgccttgcacctccaataggttttattattttttttaaattaatgaacatatgtaatattaatagttattatttactggtgcagatggttgacatttttccctattttcctcactttatggaagagttaaaacatttctaaaccagaggacaaaaggggttaatgttactttaaaattacattctatatatatataaatatatataaatatatattaaaataccagttttttttctctgggtgcaaagatgttcattcttttaaaaaatgtttaaaaaaaaaaaaaaactgcctttcttcccctcaagtcaacttttgtgctccagaaaattttctattctgtaagtctgagcgtaaaacttcaagtattaaaataatttgtacatgtagagagaaaaatgactttttcaaaaatatacaggggcagctgccaaattgatgtattatatattgtggtttctgtttcttgaaagaatttttttcgttatttttacatctaacaaagtaaaaaaattaaaaagagggtaagaaacgattccggtgggatgattttaacatgcaaaatgtccctgggggtttcttctttgcttgctttcttcctccttaccctaccccccactcacacacacacacacacacacacacacacacacacacacacactttctataaaacttgaaaatagcaaaaaccctcaactgttgtaaatcatgcaattaaagttgattacttataaatatgaactttggatcactgtatagactgttaaatttgatttcttattacctattgttaaataaactgtgtgagacagaca

SEQ ID NO: 3 - protein, 2191 aa, Homo sapiens
MLSYPEWTCWRGSHQMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

SEQ ID NO: 4 - DNA, 9674 bp, Homo sapiens
cagatgacagttgaaggaagcttcttgcaaatcagaaatgtgcttaatatttatcgagctaccatcttgcctagattaagtcatttgaactcgaaattgagtctggtttgtggacttgcagaagaattagtgtctcagttcacttaagtagagcacctagtggtgacaagaatgtgattgctttccaaaaggtgagaaatgtcacctaggaggactacatggggaaggaaatcacctgcgtatgaaatgcacagagctagaactttctggttatactcctttggtttttatttgtgtttctgtacaggcatttcagcagaaagggccagttgtgttgagtgttgatttgtttgtagcctagacttttagagctgaaagaaataatacgatccatcttgttcaagacactcatcttacaggcgaggagtttgaagtccatagaaggaatgttaagttacccagagtggacatgctggcgtggcagtcaccagatgggaataactgggaacacaagtccatttggacagccctttagtcaagctggagggcagccaatgggagccactggagtgaacccccagttagccagcaaacagagcatggtcaacagtttgcccaccttccctacagatatcaagaatacttcagtcaccaacgtgccaaatatgtctcagatgcaaacatcagtgggaattgtacccacacaagcaattgcaacaggccccactgcagatcctgaaaaacgcaaactgatacagcagcagctggttctactgcttcatgctcataagtgtcagagacgagagcaagcaaacggagaggttcgggcctgctcgctcccgcattgtcgaaccatgaaaaacgttttgaatcacatgacgcattgtcaggctgggaaagcctgccaagttgcccattgtgcatcttcacgacaaatcatctctcattggaagaactgcacacgacatgactgtcctgtttgcctccctttgaaaaatgccagtgacaagcgaaaccaacaaaccatcctggggtctccagctagtggaattcaaaacacaattggttctgttggcacagggcaacagaatgccacttctttaagtaacccaaatcccatagaccccagctccatgcagcgagcctatgctgctctcggactcccctacatgaaccagccccagacgcagctgcagcctcaggttcctggccagcaaccagcacagcctcaaacccaccagcagatgaggactctcaaccccctgggaaataatccaatgaacattccagcaggaggaataacaacagatcagcagcccccaaacttgatttcagaatcagctcttccgacttccctgggggccacaaacccactgatgaacgatggctccaactctggtaacattggaaccctcagcactataccaacagcagctcctccttctagcaccggtgtaaggaaaggctggcacgaacatgtcactcaggacctgcggagccatctagtgcataaactcgtccaagccatcttcccaacacctgatcccgcagctctaaaggatcgccgcatggaaaacctggtagcctatgctaagaaagtggaaggggacatgtacgagtctgccaacagcagggatgaatattatcacttattagcagagaaaatctacaagatacaaaaagaactagaagaaaaacggaggtcgcgtttacataaacaaggcatcttggggaaccagccagccttaccagccccgggggctcagccccctgtgattccacaggcacaacctgtgagacctccaaatggacccctgtccctgccagtgaatcgcatgcaagtttctcaagggatgaattcatttaaccccatgtccttggggaacgtccagttgccacaagcacccatgggacctcgtgcagcctccccaatgaaccactctgtccagatgaacagcatgggctcagtgccagggatggccatttctccttcccgaatgcctcagcctccgaacatgatgggtgcacacaccaacaacatgatggcccaggcgcccgctcagagccagtttctgccacagaaccagttcccgtcatccagcggggcgatgagtgtgggcatggggcagccgccagcccaaacaggcgtgtcacagggacaggtgcctggtgctgctcttcctaaccctctcaacatgctggggcctcaggccagccagctaccttgccctccagtgacacagtcaccactgcacccaacaccgcctcctgcttccacggctgctggcatgccatctctccagcacacgacaccacctgggatgactcctccccagccagcagctcccactcagccatcaactcctgtgtcgtcttccgggcagactcccaccccgactcctggctcagtgcccagtgctacccaaacccagagcacccctacagtccaggcagcagcccaggcccaggtgaccccgcagcctcaaaccccagttcagcccccgtctgtggctacccctcagtcatcgcagcaacagccgacgcctgtgcacgcccagcctcctggcacaccgctttcccaggcagcagccagcattgataacagagtccctaccccctcctcggtggccagcgcagaaaccaattcccagcagccaggacctgacgtacctgtgctggaaatgaagacggagacccaagcagaggacactgagcccgatcctggtgaatccaaaggggagcccaggtctgagatgatggaggaggatttgcaaggagcttcccaagttaaagaagaaacagacatagcagagcagaaatcagaaccaatggaagtggatgaaaagaaacctgaagtgaaagtagaagttaaagaggaagaagagagtagcagtaacggcacagcctctcagtcaacatctccttcgcagccgcgcaaaaaaatctttaaaccagaggagttacgccaggccctcatgccaaccctagaagcactgtatcgacaggacccagagtcattacctttccggcagcctgtagatccccagctcctcggaattccagactattttgacatcgtaaagaatcccatggacctctccaccatcaagcggaagctggacacagggcaataccaagagccctggcagtacgtggacgacgtctggctcatgttcaacaatgcctggctctataatcgcaagacatcccgagtctataagttttgcagtaagcttgcagaggtctttgagcaggaaattgaccctgtcatgcagtcccttggatattgctgtggacgcaagtatgagttttccccacagactttgtgctgctatgggaagcagctgtgtaccattcctcgcgatgctgcctactacagctatcagaataggtatcatttctgtgagaagtgtttcacagagatccagggcgagaatgtgaccctgggtgacgacccttcacagccccagacgacaatttcaaaggatcagtttgaaaagaagaaaaatgataccttagaccccgaacctttcgttgattgcaaggagtgtggccggaagatgcatcagatttgcgttctgcactatgacatcatttggccttcaggttttgtgtgcgacaactgcttgaagaaaactggcagacctcgaaaagaaaacaaattcagtgctaagaggctgcagaccacaagactgggaaaccacttggaagaccgagtgaacaaatttttgcggcgccagaatcaccctgaagccggggaggtttttgtccgagtggtggccagctcagacaagacggtggaggtcaagcccgggatgaagtcacggtttgtggattctggggaaatgtctgaatctttcccatatcgaaccaaagctctgtttgcttttgaggaaattgacggcgtggatgtctgcttttttggaatgcacgtccaagaatacggctctgattgcccccctccaaacacgaggcgtgtgtacatttcttatctggatagtattcatttcttccggccacgttgcctccgcacagccgtttaccatgagatccttattggatatttagagtatgtgaagaaattagggtatgtgacagggcacatctgggcctgtcctccaagtgaaggagatgattacatcttccattgccacccacctgatcaaaaaatacccaagccaaaacgactgcaggagtggtacaaaaagatgctggacaaggcgtttgcagagcggatcatccatgactacaaggatattttcaaacaagcaactgaagacaggctcaccagtgccaaggaactgccctattttgaaggtgatttctggcccaatgtgttagaagagagcattaaggaactagaacaagaagaagaggagaggaaaaaggaagagagcactgcagccagtgaaaccactgagggcagtcagggcgacagcaagaatgccaagaagaagaacaacaagaaaaccaacaagaacaaaagcagcatcagccgcgccaacaagaagaagcccagcatgcccaacgtgtccaatgacctgtcccagaagctgtatgccaccatggagaagcacaaggaggtcttcttcgtgatccacctgcacgctgggcctgtcatcaacaccctgccccccatcgtcgaccccgaccccctgctcagctgtgacctcatggatgggcgcgacgccttcctcaccctcgccagagacaagcactgggagttctcctccttgcgccgctccaagtggtccacgctctgcatgctggtggagctgcacacccagggccaggaccgctttgtctacacctgcaacgagtgcaagcaccacgtggagacgcgctggcactgcactgtgtgcgaggactacgacctctgcatcaactgctataacacgaagagccatgcccataagatggtgaagtgggggctgggcctggatgacgagggcagcagccagggcgagccacagtcaaagagcccccaggagtcacgccggctgagcatccagcgctgcatccagtcgctggtgcacgcgtgccagtgccgcaacgccaactgctcgctgccatcctgccagaagatgaagcgggtggtgcagcacaccaagggctgcaaacgcaagaccaacgggggctgcccggtgtgcaagcagctcatcgccctctgctgctaccacgccaagcactgccaagaaaacaaatgccccgtgcccttctgcctcaacatcaaacacaagctccgccagcagcagatccagcaccgcctgcagcaggcccagctcatgcgccggcggatggccaccatgaacacccgcaacgtgcctcagcagagtctgccttctcctacctcagcaccgcccgggacccccacacagcagcccagcacaccccagacgccgcagccccctgcccagccccaaccctcacccgtgagcatgtcaccagctggcttccccagcgtggcccggactcagccccccaccacggtgtccacagggaagcctaccagccaggtgccggcccccccacccccggcccagccccctcctgcagcggtggaagcggctcggcagatcgagcgtgaggcccagcagcagcagcacctgtaccgggtgaacatcaacaacagcatgcccccaggacgcacgggcatggggaccccggggagccagatggcccccgtgagcctgaatgtgccccgacccaaccaggtgagcgggcccgtcatgcccagcatgcctcccgggcagtggcagcaggcgccccttccccagcagcagcccatgccaggcttgcccaggcctgtgatatccatgcaggcccaggcggccgtggctgggccccggatgcccagcgtgcagccacccaggagcatctcacccagcgctctgcaagacctgctgcggaccctgaagtcgcccagctcccctcagcagcaacagcaggtgctgaacattctcaaatcaaacccgcagctaatggcagctttcatcaaacagcgcacagccaagtacgtggccaatcagcccggcatgcagccccagcctggcctccagtcccagcccggcatgcaaccccagcctggcatgcaccagcagcccagcctgcagaacctgaatgccatgcaggctggcgtgccgcggcccggtgtgcctccacagcagcaggcgatgggaggcctgaacccccagggccaggccttgaacatcatgaacccaggacacaaccccaacatggcgagtatgaatccacagtaccgagaaatgttacggaggcagctgctgcagcagcagcagcaacagcagcagcaacaacagcagcaacagcagcagcagcaagggagtgccggcatggctgggggcatggcggggcacggccagttccagcagcctcaaggacccggaggctacccaccggccatgcagcagcagcagcgcatgcagcagcatctccccctccagggcagctccatgggccagatggcggctcagatgggacagcttggccagatggggcagccggggctgggggcagacagcacccccaacatccagcaagccctgcagcagcggattctgcagcaacagcagatgaagcagcagattgggtccccaggccagccgaaccccatgagcccccagcaacacatgctctcaggacagccacaggcctcgcatctccctggccagcagatcgccacgtcccttagtaaccaggtgcggtctccagcccctgtccagtctccacggccccagtcccagcctccacattccagcccgtcaccacggatacagccccagccttcgccacaccacgtctcaccccagactggttccccccaccccggactcgcagtcaccatggccagctccatagatcagggacacttggggaaccccgaacagagtgcaatgctcccccagctgaacacccccagcaggagtgcgctgtccagcgaactgtccctggtcggggacaccacgggggacacgctagagaagtttgtggagggcttgtagcattgtgagagcatcaccttttccctttcatgttcttggaccttttgtactgaaaatccaggcatctaggttctttttattcctagatggaactgcgacttccgagccatggaagggtggattgatgtttaaagaaacaatacaaagaatatatttttttgttaaaaaccagttgatttaaatatctggtctctctctttggtttttttttggcgggggggtggggggggttcttttttttccgttttgtttttgtttggggggaggggggttttgtttggattctttttgtcgtcattgctggtgactcatgcctttttttaacgggaaaaacaagttcattatattcatattttttatttgtattttcaagactttaaacatttatgtttaaaagtaagaagaaaaataatattcagaactgattcctgaaataatgcaagcttataatgtatcccgataactttgtgatgtttcgggaagatttttttctatagtgaactctgtgggcgtctcccagtattaccctggatgataggaattgactccggcgtgcacacacgtacacacccacacacatctatctatacataatggctgaagccaaacttgtcttgcagatgtagaaattgttgctttgtttctctgataaaactggttttagacaaaaaatagggatgatcactcttagaccatgctaatgttactagagaagaagccttcttttctttcttctatgtgaaacttgaaatgaggaaaagcaattctagtgtaaatcatgcaagcgctctaattcctataaatacgaaactcgagaagattcaatcactgtatagaatggtaaaataccaactcatttcttatatcatattgttaaataaactgtgtgcaacagacaaaaagggtggtccttcttgaattcatgtacatggtattaacacttagtgttcggggttttttgttatgaaaatgctgttttcaacattgtatttggactatgcatgtgttttttccccattgtatataaagtaccgcttaaaattgatataaattactgaggtttttaacatgtattctgttctttaagatccctgtaagaatgtttaaggtttttatttatttatatatattttttgagtctgttctttgtaagacatggttctggttgttcgctcatagcggagaggctggggctgcggttgtggttgtggcggcgtgggtggtggctgggaactgtggcccaggcttagcggccgcccggaggcttttcttcccggagactgaggtgggcgactgaggtgggcggctcagcgttggccccacacattcgaggctcacaggtgattgtcgctcacacagttagggtcgtcagttggtctgaaactgcatttggcccactcctccatcctccctgtccgtcgtagctgccacccccagaggcggcgcttcttcccgtgttcaggcggctccccccccccgtacacgactcccagaatctgaggcagagagtgctccaggctcgcgaggtgctttctgacttccccccaaatcctgccgctgccgcgcagcatgtcccgtgtggcgtttgaggaaatgctgagggacagacaccttggagcaccagctccggtccctgttacagtgagaaaggtcccccacttcgggggatacttgcacttagccacatggtcctgcctcccttggagtccagttccaggctcccttactgagtgggtgagacaagttcacaaaaaccgtaaaactgagaggaggaccatgggcaggggagctgaagttcatcccctaagtctaccacccccagcacccagagaacccactttatccctagtcccccaacaaaggctggtctaggtgggggtgatggtaattttagaaatcacgccccaaatagcttccgtttgggcccttacattcacagataggttttaaatagctgaatacttggtttgggaatctgaattcgaggaacctttctaagaagttggaaaggtccgatctagttttagcacagagctttgaaccttgagttataaaatgcagaataattcaagtaaaaataagaccaccatctggcacccctgaccagcccccattcaccccatcccaggaggggaagcacaggccgggcctccggtggagattgctgccactgctcggcctgctgggttcttaacctccagtgtcctcttcatcttttccacccgtagggaaaccttgagccatgtgttcaaacaagaagtggggctagagcccgagagcagcagctctaagcccacactcagaaagtggcgccctcctggttgtgcagccttttaatgtgggcagtggaggggcctctgtttcaggttatcctggaattcaaaacgttatgtaccaacctcatcctctttggagtctgcatcctgtgcaaccgtcttgggcaatccagatgtcgaaggatgtgaccgagagcatggtctgtggatgctaaccctaagtttgtcgtaaggaaatttctgtaagaaacctggaaagccccaacgctgtgtctcatgctgtatacttaagaggagaagaaaaagtcctatatttgtgatcaaaaagaggaaacttgaaatgtgatggtgtttataataaaagatggtaaaacta

SEQ ID NO: 5 - protein, 398 aa, Homo sapiens
MAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFRYAWTVAQCKICASHIGWKFTATKKDMSPQKFWGLTRSALLPTIPDTEDEISPDKVILCL

SEQ ID NO: 6 - DNA, 3586 bp, Homo sapiens
gcgggtaaacagacatggccggcgaaggagatcagcaggacgctgcgcacaacatgggcaaccacctgccgctcctgcctgcagagagtgaggaagaagatgaaatggaagttgaagaccaggatagtaaagaagccaaaaaaccaaacatcataaattttgacaccagtctgccgacatcacatacatacctaggtgctgatatggaagaatttcatggcaggactttgcacgatgacgacagctgtcaggtgattccagttcttccacaagtgatgatgatcctgattcccggacagacattacctcttcagctttttcaccctcaagaagtcagtatggtgcggaatttaattcagaaagatagaacctttgctgttcttgcatacagcaatgtacaggaaagggaagcacagtttggaacaacagcagagatatatgcctatcgagaagaacaggattttggaattgagatagtgaaagtgaaagcaattggaagacaaaggttcaaagtccttgagctaagaacacagtcagatggaatccagcaagctaaagtgcaaattcttcccgaatgtgtgttgccttcaaccatgtctgcagttcaattagaatccctcaataagtgccagatatttccttcaaaacctgtctcaagagaagaccaatgttcatataaatggtggcagaaataccagaagagaaagtttcattgtgcaaatctaacttcatggcctcgctggctgtattccttatatgatgctgagaccttaatggacagaatcaagaaacagctacgtgaatgggatgaaaatctaaaagatgattctcttccttcaaatccaatagatttttcttacagagtagctgcttgtcttcctattgatgatgtattgagaattcagctccttaaaattggcagtgctatccagcgacttcgctgtgaattagacattatgaataaatgtacttccctttgctgtaaacaatgtcaagaaacagaaataacaaccaaaaatgaaatattcaggtatgcctggactgttgcccagtgtaagatctgtgcaagccatattggatggaagtttacggccaccaaaaaagacatgtcacctcaaaaattttggggcttaacgcgatctgctctgttgcccacgatcccagacactgaagatgaaataagtccagacaaagtaatactttgcttgtaaacagatgtgatagagataaagttatctaacaaattggttatattctaagatctgctttggaaattattgcctctgatacatacctaagtaaacataacattaatacctaagtaaacataacattacttggagggttgcagtttctaagtgaaactgtatttgaaacttttaagtatactttaggaaacaagcatgaacggcagtctagaataccagaaacatctacttgggtagcttggtgccattatcctgtggaatctgatatgtctggtagcatgtcattgatgggacatgaagacatctttggaaatgatgagattatttcctgtgttaaaaaaaaaaaaaatcttaaattcctacaatgtgaaactgaaactaataatttgatcctgatgtatgggacagcgtatctgtaccagtgctctaaataacaaaagctagggtgacaagtacatgttccttttggaaagaagcaaggcaatgtatattaattattctaaaagggctttgttcctttccattttctttaacttctctgagatactgatttgtaaattttgaaaattagttaaaatatgcagttttttgagcccacgaatagttgtcatttcctttatgtgcctgttagtaaaaagtagtattgtgtatttgctcagtatctgaactataagcccatttatactgttccatacaaaagctatttttcaaaaattaatttgaaccaaaactactactatagggaaaagatgccaaaacatgtcccctcacccagactaaacttgatactgtattattttgttcaatgtaaattgaagaaaatctgtaagtaagtaaaccttaagtgtgaaactaaacatgttctttgttcaaataatgtaaaatatctactcataattttaaagttctaaaaaggctgctttgccacctacctctttgcttatattcaagggtttagtgggtccttccttccacatttgtatgataactttcgttttattggtagtcctttgctactttaaaactaatcaggttaaatgtttactcaaatgtagtaatagtatgaggccaaaatacatgccttgaaggtggagtgaaatcagttgagtgtcacactgcatatttatgtaaatagagtaaatgaaaattcaagaatacttccaatcaggatatgcccctgccttattttttctaaggtctacttttactactttaataattttgggtttgtgtgacttcactggtctggtttgttcctatagttcttacactgcactaaaacttacttactccaggaagttcctgtcagtagcatagtacctctctgtaggctgaactctaaaccagttgcaatgagttaccttgtccctctgaaaagttaaaatttcagtatgtcccataccgtttactagcaaataaaattttctgatctgtaaaaacacacatatactatactactactgaaaatggttttacacatgacaaaaccagacctgatagctaaatctgcatactttctatacagaaatagtatgtattatatgacgttgggtctaaatctgtattttggcagtacttaaatattttaagtaagtatttcaaagtattattttataatgcatctaattactgacctgtatgcagtcatttctgaggctttcttgcatcatagcccctgtgacatttcctcttagaaatattacactctacaaaattgttttatcaaggtccaaaattactatttgctcatagagtacaaagatgttatgactggctacacagaaaggaaataaattatgaaatgtcagtatagcagctgaaattatagcagctgaaataaacagtttgtattatacattttatttacctgatgaaagcatttaggactcaaatctttagagaatatataataatagccatatccttttaattaataaaataacactttaaagacaggcatattaccattgtatatgacacctaatacacattgtcagataccacataaacatatttatcttccaataccaatgtttgttttattaaaacaataattaattaataggctcaatagtgaccctcaagttaatcccatgacttccccaggctctgcatgcataaagaaatcattgtgacagaaaaatatcaaccacatactgaagcatagcttgctagaacaaatcctgtacatatgaatgccagcaactggggacacatttagtattcacattttatgcaaacttacttggtcaattatttcttttttaagcacaggtttattttggaattcagataagaactgacacatcatagacagtcattgttaaggtaaccctgttatttcaattgcaaacaaacaataaacgttttttcttcaa